SEQUENCE LISTING (3 sequences)

SEQ ID NO: 1 - protein, 1404 aa, Homo sapiens
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 2 - DNA, 5041 bp, Homo sapiens
gcggccgcgactattcggtacctgaaaacaacgatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaatttggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccgctcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctctgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaatgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagactgatgagcaaaggaggagtcaactaatgaagaaatgaataataaattttgacactgaaaaacattttattaataaagaatattgacatgagtataccagtttatatataaaaatgtttttaaacttgacaatcattacactaaaacagatttgataatcttattcacagttgttattgtttacagaccatttaattaatatttcctctgtttattcctcctctccctcccattgcatggctcacacctgtaaaagaaaaaagaatcaaattgaatatatcttttaagaattcaaaactagtgtattcacttaccctagttcattataaaaaatatctaggcattgtggatataaaactgttgggtattctacaacttcaatggaaattattacaagcagattaatccctctttttgtgacacaagtacaatctaaaagttatattggaaaacatggaaatattaaaattttacacttttactagctaaaacataatcacaaagctttatcgtgttgtataaaaaaattaacaatataatggcaataggtagagatacaacaaatgaatataacactataacacttcatattttccaaatcttaatttggatttaaggaagaaatcaataaatataaaatataagcacatatttattatatatctaaggtatacaaatctgtctacatgaagtttacagattggtaaatatcacctgctcaacatgtaattatttaataaaactttggaacattaaaaaaataaattggaggcttaaaaaaaaaaaaaaaaa

SEQ ID NO: 3 - protein, 7 aa, Homo sapiens
KEPAPTT

(19) United States
(12) Patent Application Publication
(10) Pub. No.: US 2022/0332839 A1
(43) Pub. Date: Oct. 20, 2022

(54) USE OF PRG4 TO TREAT CANCER

(71) Applicant: Lubris LLC, Naples, FL (US)

(72) Inventors: Shirin Bonni, Calgary (CA); Anusi Sarkar, Calgary (CA); Tannin Schmidt, Avon, CT (US); Benjamin Sullivan, Concord, MA (US)

(21) Appl. No.: 17/596,066

(22) PCT Filed: Jun. 3, 2020
(86) PCT No.: PCT/US2020/035841

§ 371 Date: Dec. 2, 2021

Related U.S. Application Data
(60) Provisional application No. 63/013,427, filed on Apr. 21, 2020.
(60) Provisional application No. 62/856,514, filed on Jun. 3, 2019.

(51) Int. Cl. C07K 16/28; A61K 31/44; A61P 35/04

(52) U.S. Cl. CPC C07K 16/2884; A61K 31/44; A61P 35/04

(57) ABSTRACT

Disclosed herein are methods of using PRG4 glycoprotein, also known as lubricin, to treat or prevent cancer.

### CROSS-REFERENCE TO RELATED APPLICATIONS

This application claims priority to and the benefit of U.S. Provisional Patent Application No. 62/856,514 filed Jun. 3, 2019, and of U.S. Provisional Patent Application No. 63/013,427 filed Apr. 21, 2020, the contents of each of which are incorporated by reference herein in their entireties.

### SEQUENCE LISTING

The instant application contains a Sequence Listing which has been submitted electronically in ASCII format and is hereby incorporated by reference in its entirety. Said ASCII copy, created on Jun. 2, 2020, is named LUB_032WO_SL.txt and is 19,246 bytes in size.

### FIELD OF THE INVENTION

This invention relates to new uses of the human glycoprotein PRG4, also known as lubricin. More particularly, it relates to using PRG4 to treat cancer and conditions related to or incident to cancer.

### BACKGROUND

The proteoglycan 4 gene (PRG4) encodes megakaryocyte stimulating factor (MSF) as well as highly glycosylated differently spliced variant and glycoforms of “superficial zone protein” also known as lubricin. Superficial zone protein was first localized at the surface of explant cartilage from the superficial zone and identified in conditioned medium. Lubricin was first isolated from synovial fluid and demonstrated lubricating ability in vitro similar to synovial fluid at a cartilage-glass interface and in a latex-glass interface. It was later identified as a product of synovial fibroblasts, and its lubricating ability was discovered to be dependent on O-linked β (1-3) Gal-GalNAc oligosaccharides within a large mucin like domain of 940 amino acids encoded by exon 6. Lubricin molecules are differentially glycosylated and several naturally occurring splice variants have been reported. They are collectively referred to herein as PRG4. PRG4 has been shown to be present inside the body at the surface of synovium, tendon, articular cartilage such as meniscus, and in the protective film of the eye, among other sites, and plays an important role in joint lubrication and synovial homeostasis.

Full-length recombinant human PRG4 (rhPRG4) protein has been expressed successfully at large scale making it available for basic and translational-based investigations. rhPRG4 has been shown to retain appropriate higher order structure and glycosylations, and thus displays efficient in vitro lubricating and anti-adhesive function (Abubacker et al., Ann Biomed Eng. 2016; 44(4):1128-37; Samsom et al., Exp Eye Res. 2014; 127:14-9). Importantly, rhPRG4 provides effective in vivo therapeutic value in preservation of joint health via intra-articular injection in preclinical in vivo osteoarthritis models (Elsaid et al., Osteoarthritis and Cartilage. 2015; 23(1):114-21; Walker et al., Am. J. Sports Med., 2017; 45(7):1512-21). Applicants have now discovered that lubricin has an effect on cancer cells and can be used to treat cancer and conditions incident to cancer. In particular, Applicants have determined that PRG4 can alter the phenotype of neoplastic cells as well as the stress response of neoplastic cells, for example, making tumor cells less invasive and less migratory.

### SUMMARY OF THE INVENTION

The invention includes in one aspect a method of treating cancer or slowing the growth or progression of a cancer in a patient where PRG4 is administered to the patient to treat the cancer or to slow the growth or progression of the cancer.

In one embodiment, the PRG4 is recombinant human PRG4 comprising the amino acid sequence of residues 25-1404 of SEQ ID NO:1. In one embodiment, the PRG4 has at least 99% sequence identity with residues 25-1404 of SEQ ID NO:1. In another embodiment, the PRG4 has at least 99.5% sequence identity with residues 25-1404 of SEQ ID NO:1.

In one embodiment, the cancer is selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma (HCC), Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor. For example, the cancer may be breast cancer. For example, the cancer may be head or neck cancer, breast cancer, pancreatic cancer, gastrointestinal cancer, colorectal cancer, prostate cancer, colon cancer, bladder cancer, or leukemia. For example, the cancer may be hepatocellular carcinoma.

In one embodiment, the PRG4 is administered in connection with another anti-cancer agent. For example, the anti-cancer agent may be chemotherapy or radiologic treatment. The radiologic treatment may be, for example, external beam radiation therapy, brachytherapy, or stereotactic body radiation therapy (SBRT).

In one embodiment, the PRG4 is administered in connection with another anti-cancer agent, where the anti-cancer agent is administered at a dose that is less than the therapeutically effective dose of the anti-cancer agent to treat the cancer administered alone without PRG4. For example, in one embodiment, the anti-cancer agent is sorafenib administered at a dose less than 800 mg per day or less than 400 mg twice daily. In another embodiment, the anti-cancer agent is regorafenib administered at a dose less than 160 mg daily. In another embodiment, the cancer is hepatocellular carcinoma.

In one embodiment, the chemotherapy may be selected from actinomycin, abraxane, altretamine, aranose, azacitidine, azathioprine, bendamustine, bleomycin, bortezomib, busulfan, cabazitaxel, capecitabine, carboplatin, carmofur, carmustine, chlorambucil, chlormethine, chlorozotocin, cisplatin, cladribine, clofarabine, crizotinib, cyclophosphamide, cytarabine, dacarbazine, dactinomycin, dasatinib, daunorubicin, decitabine, docetaxel, doxifluridine, doxorubicin, epirubicin, ertramustine, ethylnitrosourea, erlotinib, etoposide, floxuridine, fludarabine, fluorouracil, fotemustine, gefitinib, gemcitabine, hydroxyurea, hydroxycarbamide, idarubicin, ifosfamide, irinotecan, imatinib, ixabepilone, lapatinib, lomustine, mechlorethamine, melphalan, mercaptopurine, methotrexate, mitomycin, mitoxantrone, nedaplatin, nelarabine, nimustine, nilotinib, N-nitroso-N-methylurea, procarbazine, oxaliplatin, paclitaxel, pemetrexed, pentostatin, ranimustine, raltitrexed, regorafenib, romidepsin, semustine, sorafenib, streptozotocin, tafluposide, tamoxifen, taxotere, tegafur, temozolomide, temsirolimus, teniposide, tioguanine, tofacitinib, opotecan, valrubicin, vemurafenib, vinblastine, vincristine, vincristine, vindesine, vinflunine, vinorelbine, vorinostat, or vismodegib. In one embodiment, the chemotherapy is sorafenib and/or regorafenib. In a further embodiment, the cancer is hepatocellular carcinoma and chemotherapy is sorafenib and/or regorafenib.

In another embodiment, the chemotherapy may be an antibody treatment selected from alemtuzumab, bevacizumab, blinatumomab, brentuximab, certolizumab, cetuximab, daratumumab, dinutuximab, ibritumomab, obinutuzumab, ofatumumab, olaratumab, panitumumab, pertuzumab, ramucirumab, rituximab, siltuximab, trastuzumab, rituximab, inotuzumab, gemtuzumab, bevacizumab, camiplimab, or spartalizumab.

According to one embodiment, wherein the PRG4 is administered systemically to the patient, whereas in other embodiments PRG4 is administered by subcutaneous, intramuscular, or intravenous administration. PRG4 may also be administered locally to the location of the cancer. Administration may be by injection. PRG4 may be administered in an amount of 0.1 μg/kg to 4,000 μg/kg.

In some embodiments, the PRG4 enhances chemosensitivity of the cancer to the anti-cancer agent. In some embodiments, the combination of PRG4 and the anti-cancer agent treats the cancer.

In another aspect, the invention provides a method for preventing or inhibiting recurrence of a previously treated cancer. The method includes administering to a patient in need thereof a therapeutically effective amount of PRG4 to prevent recurrence or growth of a previously treated cancer in the patient. In one embodiment, the PRG4 is recombinant human PRG4 comprising the amino acid sequence of residues 25-1404 of SEQ ID NO:1 In one embodiment, the PRG4 has at least 99% sequence identity with residues 25-1404 of SEQ ID NO:1. In another embodiment, the PRG4 has at least 99.5% sequence identity with residues 25-1404 of SEQ ID NO:1.

In some embodiments, the previously treated cancer is the cancer is selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma, Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor. For example, in one embodiment, the cancer is breast cancer. In a further embodiment, the cancer is triple negative breast cancer. In yet another embodiment, the cancer is head or neck squamous cell carcinoma, breast cancer, pancreatic cancer, gastrointestinal cancer, colorectal cancer, prostate cancer, colon cancer, bladder cancer, or leukemia. In one embodiment, the cancer is hepatocellular carcinoma.

In some embodiments, the PRG4 is administered locally to the site of the cancer that was previously treated. For example, local administration can occur by topical administered or by injection to the site. In other embodiments, the PRG4 is administered systemically to the patient. PRG4 may be administered in amount of 0.1 μg/kg to 4,000 μg/kg.

In some embodiments, the previously treated cancer was removed from the patient by surgical resection and PRG4 is administered to the patient after the surgical resection of the tumor. In such instances, PRG4 may be administered locally to the site of the surgical resection of the tumor. PRG4 may be administered in amount of 0.1 μg/kg to 4,000 μg/kg.

According to some embodiments of the method, the patient is in complete remission from the previously treated cancer, whereas in other embodiments, the patient is in partial remission from the previously treated cancer. In some embodiments, the recurrence of the previously treated cancer is prevented for one year, two years, three years, four years or five years by administration of the PRG4.

In another aspect, the invention includes a method of treating cancer involving administering to a patient in need thereof PRG4 in combination with an immunotherapy agent, wherein the combination of the PRG4 and the immunotherapy treats the cancer. In one embodiment, the immunotherapy is an anti-PD1 or anti-PD-L1 antibody. In another embodiment, the immunotherapy is selected from atezolizumab, avelumab, durvalumab, pembrolizumab, nivolumab, cemiplimab, ipilimumab. In a further embodiment, treatment of the patient with PRG4 and an immunotherapy agent improves the treatment of the cancer as compared to treatment with the immunotherapy alone.

According to this method of the invention, the cancer may be selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma (HCC), Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor. The cancer may be breast cancer in one embodiment, triple negative breast cancer in another embodiment, or the cancer may be head or neck cancer, breast cancer, pancreatic cancer, gastrointestinal cancer, colorectal cancer, prostate cancer, colon cancer, bladder cancer, or leukemia. In one embodiment, the cancer is hepatocellular carcinoma.

According to one embodiment, the PRG4 is administered to the patient systemically, whereas in another embodiment, the PRG4 is administered to the patient locally at a site of a cancer. In one embodiment, the PRG4 is administered in amount of 0.1 μg/kg to 4,000 μg/kg. In yet another embodiment, the PRG4 is recombinant human PRG4 comprising the amino acid sequence of residues 25-1404 of SEQ ID NO:1. In one embodiment, the PRG4 has at least 99% sequence identity with residues 25-1404 of SEQ ID NO:1. In another embodiment, the PRG4 has at least 99.5% sequence identity with residues 25-1404 of SEQ ID NO:1.

In one embodiment, the invention comprises PRG4 for use in treating cancer. For example, in one embodiment, the PRG4 is recombinant human PRG4 comprising the amino acid sequence of residues 25-1404 of SEQ ID NO:1. In one embodiment, the PRG4 has at least 99% sequence identity with residues 25-1404 of SEQ ID NO:1. In another embodiment, the PRG4 has at least 99.5% sequence identity with residues 25-1404 of SEQ ID NO:1.

### BRIEF DESCRIPTION OF THE DRAWINGS

FIGS. 1A-C are bar graphs presenting data demonstrating the binding of recombinant human proteoglycan 4 (rhPRG4), high-molecular weight hyaluronic acid (HMW HA), and medium molecular weight hyaluronic acid (MMW HA) to recombinant human CD44 receptor as detected by TMB-ELISA at 450 nm. Data represents the average of 4 independent experiments with triplicate wells per group. FIG. 1A depicts binding of rhPRG4, HMW HA, MMW HA and vitronectin to CD44-IgG1Fc and using IgG1 Fc. The star (*) indicates that the 450 nm absorbance in the CD44-IgG1 Fc wells were statistically significantly higher (p<0.001) than the IgG1 Fc wells for rhPRG4, HMW HA and MMW HA. FIG. 1B shows the concentration-dependent CD44 binding of rhPRG4, HMW HA and MMW HA. CD44 binding to rhPRG4 was significantly higher than to HMW HA or MMW HA (p<0.001). The double stars (**) indicate that CD44 binding to rhPRG4 was significantly higher than to MMW HA (p<0.001). FIG. 1C depicts the competition between rhPRG4 (5 μg/mL) and either HMW HA or MMW HA (0.01 μg/mL to 50 μg/mL) on binding to CD44 coated on 96-well ELISA plates. The star (*) indicates the percentage CD44 binding in HMW HA+rhPRG4 wells was significantly lower than rhPRG4 wells (p<0.05); (**) indicates the percentage CD44 binding in MMW HA+rhPRG4 wells was significantly lower than rhPRG4 wells (p<0.05).

FIGS. 2A-B depict binding of recombinant human proteoglycan 4 (rhPRG4) to recombinant CD44 and competition between rhPRG4 and high molecular weight hyaluronic acid (HMW HA) on CD44 binding using surface plasmon resonance. FIG. 2A is a sensogram depicting the concentration-dependent association and dissociation of rhPRG4 (300 μg/mL to 50 μg/mL) to immobilized CD44-IgG₁Fc. Dashed line curves represent the binding curves of rhPRG4 to CD44 chimeric protein and the black lines represent the fitted 1:1 binding model. FIG. 2B is a plot showing the relative response-HMW HA binding vs. relative response-rhPRG4 binding. Competition between rhPRG4 and HMW HA on binding to immobilized CD44-IgG₁Fc. rhPRG4 was injected at 300 (1), 250 (2), 200 (3), 150 (4), 100 (5), 50 (6) and 0 (7) μg/mL. Following dissociation of rhPRG4, HMW HA was injected at 50 μg/mL. As the concentration of rhPRG4 increased, subsequent HMW HA binding to CD44 decreased.

FIGS. 3A-B show the impact of sialidase-A and O-glycosidase digestion of recombinant human proteoglycan 4 (rhPRG4) on binding of rhPRG4 to CD44. Data represents the average of 4 independent experiments with triplicate wells per group. FIG. 3A is a bar graph depicting binding of rhPRG4, sialidase-A digested rhPRG4, O-glycosidase digested rhPRG4 and sialidase-A+O-glycosidase digested rhPRG4 to CD44. The 450 nm absorbance values across different groups were normalized to the absorbance values in the undigested rhPRG4 group. The (*) indicates that CD44 binding in the sialidase A-digested and O-glycosidase-digested rhPRG4 was significantly higher compared to undigested rhPRG4 (p<0.01). (**) indicates that CD44 binding in the Sialidase-A+0-Glycosidase digested rhPRG4 was significantly higher compared to sialidase-A digested, O-glycosidase-digested and undigested rhPRG4 (p<0.01). FIG. 3B is a photograph of an SDS-PAGE of rhPRG4, sialidase-A digested rhPRG4, O-glycosidase digested rhPRG4 and a combination of sialidase-A and O-glycosidase digested rhPRG4. The gel was stained overnight with Commassie Blue. Digestion with sialidase-A and O-glycosidase resulted in reducing the apparent molecular weight of rhPRG4.

FIGS. 4A-G: rhPRG4 suppresses TGFβ-induced invasive growth of MDA-MB-231 cell-derived organoids. FIG. 4A: Representative DIC light microscopy images of 8-day old three-dimensional MDA-MB-231 cell-derived organoids that were left untreated or incubated with 100 pM TGFβ, without or with increasing concentrations of rhPRG4 (0.1, 10 and 100 μg/mL) in complete growth medium. FIG. 4B: Bar graph depicts mean±SEM proportion of spherical organoids expressed as a percentage of total colonies counted for each experimental condition from four independent experiments including the one shown in A. FIG. 4C: Representative DIC light microscopy images of 8-day old three-dimensional MDA-MB-231 cell-derived organoids that were incubated with 10 μg/mL of mouse IgG or anti-PRG4 mAb 4D6, along with or without 100 pM TGFβ and 100 μg/mL rhPRG4 in different combinations in complete growth medium. FIG. 4D: Bar graph depicts mean±SEM proportion of spherical organoids expressed as a percentage of total colonies counted for each experimental condition from three independent experiments including the one shown in C. FIG. 4E: Representative DIC light microscopy images of untreated or 100 pM TGFβ-treated 8-day old three-dimensional MDA-MB-231 cell-derived organoids using Matrigel that was mixed with vehicle or 100 μg/mL rhPRG4. FIG. 4F: Bar graph depicts mean±SEM proportion of spherical organoids expressed as a percentage of total colonies counted for each experimental condition from three independent experiments including the one shown in E. FIG. 4G: Representative fluorescence microscopy images of nuclear (Hoechst 33342, blue), actin (TRITC-phalloidin, yellow), and laminin (Rat anti-laminin/anti-rat Alexa 647, red) staining of formaldehyde-fixed 8 day old MDA-MB-231 cell-derived organoids that were left untreated or incubated with TGFβ, with or without rhPRG4, in complete growth medium. This experiment was repeated two independent times with similar outcomes. Significant difference, ANOVA: *P<0.05, **P<0.01, ***P<0.001. Scale bar indicates 50 μm. For FIGS. 4A, 4C and 4E, green and red arrows indicate spherical and invasive organoids respectively. Green arrows appear in all boxes on the top row of FIG. 4A and on the bottom row in the two boxes on the right. The two boxes in the bottom row on the left have red arrows. In FIG. 4C, on the top row, from left to right, the first, third, and fourth boxes have green arrows, whereas the second box from the left has a red arrow. In FIG. 4C, the first box on the left in the bottom row has a green arrow whereas the remaining boxes on the bottom row have red arrows. In FIG. 4E, all boxes have a green arrow except the left hand box on the bottom row, which has a red arrow. For FIG. 4G, green arrows indicate cortical actin (bottom row, middle box for both TGFβ+ and TGFβ−), yellow arrows indicate stress-fiber like actin (top row, middle box for both TGFβ+ and TGFβ−), blue arrows indicate intact laminin rings (TGFβ−, far right image in top and bottom rows as well as far right image in bottom row of TGFβ+), red arrow indicates disruption of laminin ring (TGFβ+, top row far right image), and white arrows (TGFβ+, top row far right image) indicate representative sites of laminin loss.

FIGS. 5A-D: rhPRG4 suppresses breast cancer cells' invasive and migratory abilities. FIG. 5A: Representative DIC light microscopy images of crystal violet-stained 12 h-serum-starved MDA-MB-231 cells appearing on the underside of Matrigel-coated membrane of a transwell insert, with the bottom well containing complete growth medium without (−) or with 100 pM TGFβ, alone or with 10 μM of TORT inhibitor SB435142 (KI) or 100 μg/mL of rhPRG4. Scale bar represents 150 μm. FIG. 5B: Bar graph depicts mean±SEM proportion of invaded cells counted from eight randomly chosen non-overlapping fields for each experimental conditions from three independent experiments including the one shown in A. FIG. 5C: Representative DIC light microscopy images of serum-starved MDA-MB-231 cells seeded in wells of a 12-well plate at 0 and 36 h after the introduction of a scratch, and incubated with 0.2% FBS-containing medium without (−) or with 100 pM TGFβ, alone or with 10 μM KI or 100 μg/mL rhPRG4. Scale bar represents 500 μm. FIG. 5D: Bar graph depicts mean±SEM proportion of scratch closure (%) at 36 h with respect to the 0 hour of 5 non-overlapping images of each experimental condition from three independent experiments including the one shown in C. Significant difference, ANOVA: *P<0.05, **P<0.01, ***P<0.001.

FIGS. 6A-D: rhPRG4 does not affect TGFβ-Smad signaling. FIG. 6A: phospho-Smad2 (pSmad2), total Smad2/3 (tSmad2/3) and actin immunoblots of lysates of MDA-MB-231 cells which were either left untreated (control) or incubated with 100 pM TGFβ, without or with 10 μM KI or 100 μg/mL rhPRG4 in complete growth medium for 12 h. FIG. 6B Bar graph represents the mean±SEM of the proportion of pSmad2 relative to the total protein abundance of Smad2/3 and expressed as fold change with respect to the control from four independent experiments including the one shown in A. FIG. 6C: A schematic representation of the 3TP-Lux reporter construct with three consecutive TPA (12-O-tetradecanoylphorbol 13-acetate) response elements (TREs) and a portion of the plasminogen activator inhibitor 1 (PAI-1) promoter region driving the expression of luciferase gene. TGFβ treatment triggers the phosphorylation, nuclear translocation, and binding of the Smads to 3TP promoter leading to increase in the abundance of the luciferase enzyme. FIG. 6D: MDA-MB-231 cells were transfected with the 3TP-Lux reporter construct along with a Renilla luciferase expression construct driven by a CMV promoter. Cells were left untreated (control) or incubated with 100 pM TGFβ, without or with 100 μg/mL rhPRG4 in 0.2% FBS-containing medium. Bar graph represents the 3TP promoter-driven luciferase values normalized to the Renilla luciferase expression (relative light units), and the normalized data are expressed relative to the normalized luciferase data in lysates of untreated cells. Significant difference, ANOVA: ***P≤0.001.

FIGS. 7A-E: rhPRG4 suppresses low molecular weight hyaluronic acid (LMWHA)-induced invasion of breast cancer cells. FIG. 7A: Lysates of MDA-MB-231 cells were subjected to immunoprecipitation using a CD44 antibody (CD44 IP) or a non-specific Rat IgG antibody (IgG IP) followed by CD44 immunoblotting of the immunoprecipitates. CD44 protein abundance in the lysates was also confirmed by CD44 immunoblotting (input). FIG. 7B: Representative DIC light microscopy images of 8-day old three-dimensional MDA-MB-231 cell-derived organoids incubated with growth medium without or with increasing concentrations of LMWHA (10, 100 or 400 μg/mL), alone or together with 100 μg/mL rhPRG4. Scale bar indicates 50 μm. Green arrows (Top row, two left hand images and all images in bottom row) and red arrows (top row, two right hand images) indicate spherical and invasive organoids respectively. FIG. 7C: Bar graph depicts mean±SEM proportion of spherical organoids expressed as a percentage of total colonies counted for each experimental condition from three independent experiments including the one shown in B. FIG. 7D: Representative DIC light microscopy images of crystal violet-stained 12 h-serum-starved MDA-MB-231 cells appearing on the underside of Matrigel-coated membrane of a transwell insert, with the bottom well containing complete growth medium without (−) or with 400 μg/mL LMWHA, alone or with 5 μg/mL CD44 neutralizing antibody or 100 μg/mL rhPRG4. Scale bar represents 150 μm. FIG. 7E: Bar graph depicts mean±SEM proportion of invaded cells relative to control were counted from eight randomly chosen non-overlapping fields for each experimental condition from three independent experiments including the one shown in D. ANOVA: Significant difference, ANOVA: *P<0.05, **P<0.01, ***P<0.001.

FIGS. 8A-F: CD44 is crucial for TGFβ-induced invasiveness in MDA-MB-231 cells. FIG. 8A: CD44 immunoblot of lysates of MDA-MB-231 cells transfected with the pU6 RNAi vector (vector control), or the plasmids CD44i-1, CD44i-2, alone or together (CD44i-1+2) that expresses shRNAs targeting two distinct sequences of CD44 mRNA. Actin was used as loading control. FIG. 8B: Representative CD44, GFP and nuclei fluorescence microscopy images of MDA-MB-231 cells transfected as in A, and subjected to anti-CD44 indirect immunofluorescence (Rat anti-CD44/anti-rat Alexa 647, red; color appears in bottom row of images) and counterstained with Hoechst 33342 fluorescent nucleotide dye (blue; appears in top row of images) to visualize nuclei. GFP (green; appears in middle row of images) signal indicate vector control or CD44 RNAi-1/2-transfected cells. Arrows show examples of vector (left), CD44i-1 (2^{nd} column), CD44i-2 (3^{rd} column) and CD44i-1+2 (right) transfected cells to highlight the knockdown of endogenous CD44 by the two CD44 RNAi plasmids. This experiment was repeated three times with similar outcomes. FIG. 8C: Representative DIC light microscopy images of untreated (−), 100 pM TGFβ or 400 μg/mL LMWHA-treated, 8-day old three-dimensional organoids in complete growth medium, derived from MDA-MB-231 cells, transfected with vector control or CD44i-1 and CD44i-2, individually or in combination. FIG. 8D: Bar graph depicts mean±SEM proportion of spherical organoids expressed as a percentage of total colonies counted for each experimental condition from three independent experiments including the one shown in C. FIG. 8E: CD44 and FLAG immunoblots of lysates of MDA-MB-231 cells transfected with an empty vector or CD44/FLAG expression plasmids. Actin was used as loading control. FIG. 8F: Representative DIC light microscopy images of vector control or CD44/FLAG expressing 8 day-old MDA-MB-231 cell-derived organoids grown in complete growth medium without (−) or with 100 pM TGFβ or 400 μg/mL LMWHA, alone or with 100 μg/mL rhPRG4. FIG. 8G: Bar graph depicts mean±SEM proportion of spherical organoids expressed as a percentage of total colonies counted for each experimental condition from three independent experiments including the one shown in F. Significant difference, ANOVA: *P≤0.05, **P≤0.01, ***P≤0.001. Scale bar indicates 50 μm. Green arrows and red arrows indicate spherical and invasive organoids respectively. Green arrows appear on all images in FIG. 8C except the far right images of the middle and bottom rows; red arrows appear on all images in FIG. 8F −rhPRG4 except the top row far left image which is green; green arrows appear on all images in the +rhPRG4 panel.

FIGS. 9A-D: TGFβ induces invasiveness in the breast cancer cells in a HA-CD44-dependent manner. FIG. 9A: Representative DIC light microscopy images of 8-day old three-dimensional MDA-MB-231 cell-derived organoids that were left untreated (−) or incubated with different concentrations of LMWHA (100 or 400 μg/mL), without or with 100 pM TGFβ, along with or without KI (10 μM), CD44 neutralizing antibody (2.5 μg/mL), or rhPRG4 (100 μg/mL), in complete growth medium. FIG. 9B: Bar graph depicts mean±SEM proportion of spherical organoids expressed as a percentage of total organoids counted for each experimental condition from three independent experiments including the one shown in A. FIG. 9C: Representative DIC light microscopy images of 8-day old three-dimensional MDA-MB-231 cell-derived organoids that were treated without or with 0.5 mM 4-MU, without or with 100 pM TGFβ, along with or without 400 μg/mL LMWHA, and LMWHA with 100 μg/mL rhPRG4, in complete growth medium. FIG. 9D: Bar graph depicts mean±SEM proportion of spherical organoids expressed as a percentage of total colonies counted for each experimental condition from three independent experiments including the one shown in C. Significant difference, ANOVA: **P<0.01, ***P<0.001. Scale bar indicates 50 μm. Green arrows and red arrows indicate spherical and invasive organoids respectively. In the top panel of FIG. 9A all arrows are red except in the top row, first, third and fourth images from the left and third image from the left in the second row. In the bottom panel of FIG. 9A, all arrows are green. In the top panel of FIG. 9C, all arrows are green except the arrow in the upper right image is red. In the middle panel of FIG. 9C, all arrows are red. In the lower panel of FIG. 9C, all arrows are green.

FIGS. 10A-E: rhPRG4 and TGFβ have opposing effects on the protein abundance of CD44 and HAS2. FIG. 10A: CD44 and phospho-Smad2 (pSmad2) immunoblots of lysates of MDA-MB-231 cells incubated in complete growth medium without (control) or with 100 pM TGFβ, alone or together with 10 μM KI or 100 μg/mL rhPRG4. Actin was used as loading control. FIG. 10B: Bar graph depicts mean±SEM proportion of CD44 immunoreactive band in each treatment condition from four independent experiments including the one shown in A. FIG. 10C: Representative CD44 (Rat anti-CD44/anti-rat Alexa 647, red; appears in columns labeled “CD44”), and nuclei (Hoechst, blue; appears in columns labeled “nuclei”) fluorescence microscopy images of fixed 8 day-old MDA-MB-231 cells-derived organoid that were incubated in complete growth medium without or with 100 pM TGFβ, alone or together with 100 μg/mL rhPRG4. This experiment was repeated two times with similar outcomes. Scale bar indicates 50 μm. FIG. 10D: HAS2 and phospho-Smad2 (pSmad2) immunoblots of lysates of MDA-MB-231 cells incubated in complete growth medium without (control) or with 100 pM TGFβ, alone or together with 10 μM KI or 100 μg/mL rhPRG4. Actin was used as loading control. FIG. 10E: Bar graph depicts mean±SEM proportion of HAS2 immunoreactive band in each treatment condition from three independent experiments including the one shown in D. Significant difference, ANOVA: ***P≤0.001; **P≤0.01; *P≤0.05, unpaired T test: ^{#}=0.0164.

FIG. 11 is the amino acid sequence of full length (non-truncated) human PRG4 (SEQ ID NO:1: 1404 residues). Residues 1-24 (shown in bold) represent the signal sequence and residues 25-1404 represent the mature sequence of human PRG4. The glycoprotein does not require the lead sequence in its active form.

FIG. 12 is the nucleic acid sequence for the PRG4 gene (SEQ ID NO: 2) encoding the full length 1404 AA human PRG4 protein.

FIG. 13 shows Kaplan Meier survival curves of hepatocellular carcinoma (HCC) patients based on levels of endogenous tissue mRNA expression of markers of interest (lubricin, CSPG4, VCAN, and HSPG2). The Y-axis is cumulative survival and the X-axis is survival in months. The patients are stratified as high or low according to values above or below the median mRNA expression value for any marker of interest. The data show that lubricin expression is positively correlated with HCC patients survival.

FIGS. 14A-B show lubricin protein expression in HCC and the tissue localization. FIG. 14A shows Western blot analysis and quantification of lubricin protein levels in tumoral and peritumoral paired tissues of 14 HCC patients. FIG. 14B shows the immunofluorescence of HCC tumor tissues displaying the localization of lubricin (green) and αSMA (red). Nuclei are represented by blue. In the far right panel of the bottom row, only blue and red are seen. Green and red are seen together to the largest extent in the first and second panels of the top row. Green and red are seen together to a smaller extent in the last panel of the top row and the first two panels of the bottom row.

FIG. 15 is a series of bar graphs showing that TGFβ induces lubricin expression in CAFs (top row) and ex-vivo cultured HCC tissues (bottom row). In the top row of graphs, the mRNA levels of the control, LY, TGF, and TGF+LY are shown from left to right in each graph. On the bottom row of graphs, the mRNA levels of each of LY, TGF, and TGF+LY vs control are shown from left to right in each graph. The levels of αSMA, CSPG4, HSPG2, lubricin, and VCN are shown. mRNA expression of lubricin in 48 hour-treated CAFs derived from HCC tumors (n=5), and in 48 hour-treated ex-vivo HCC tissues (n=6). T-Test: * p<0.05; ** p<0.01; *** p<0.001.

FIG. 16 shows Kaplan Meier curves showing the cumulative survival of 78 HCC cases. Patients were stratified according to values above or below the relative medians. The tumor expression of lubricin is correlated with a better prognosis in HCC with concomitant low CD44 levels. Survival curves marked with an ↑ correspond with the molecule indicated in the legend as being ↑, whereas the unmarked survival curve corresponds with the molecule in the legend as being ↓. In the top row, from left to right, the legend for the first curve shows lubricin↑ and lubricin↓, with a p=0.000; the legend for the second curve shows TGFβ1↑ and TGFβ1↓ with a p=0.084. In the bottom row, the legend for the first curve shows αSMA↑ and αSMA↓ with a p=0.637; the legend for the second curve shows CD44↑ and CD44↓ with a p=0.302. For the curve labeled “Lubricin ↑, the legend shows TGFβ1↑ and TGFβ1↓ with a p=0.478, while the curve labeled “Lubricin ↓, the legend shows TGFβ1↑ and TGFβ1↓ with a p=0.024. For the curve labeled “CD44 ↑, the legend shows Lubricin ↑ and Lubricin ↓ with a p=0.187, while the curve labeled “CD44↓, the legend shows Lubricin↑ and Lubricin ↓ with a p=0.000.

FIGS. 17A-B show that CD44 silencing in HCC cells impairs cell adhesion to rhPRG4, but not migration. In FIG. 17A, bar graphs show cell adhesion in HLE and HLF cells as a % of FN. The Y-axis is cell adhesion as a % of FN. The dark bars are for control-shRNA while the light bars are for CD44-shRNA. The results for uncoated, lubricin, and FN are shown from left to right. For the well plate images, the top row is control-shRNA and the bottom is CD44-shRNA, with the wells being for uncoated, lubricin, and FN from left to right. Cells were seeded on uncoated, or rhPRG4- or fibronectin (Fn)-coated well surfaces, and allowed to attach and spread for 30 minutes. In FIG. 17B, cells were seeded on the top of the trans-well membrane, (previously coated with Fn on the lower side) and allowed to migrate for 16 hours in the presence or not of soluble rhPRG4 in the lower chamber. T-Test: * p<0.05; ** p<0.01; *** p<0.001. The images for vehicle are found in the top row, while images for lubricin are found in the bottom row. The accompanying bar graph shows number of cells/microscopic field for vehicle versus lubricin for control-shRNA (HLE), CD44-shRNA (HLE), control-shRNA (HLF), and CD44-shRNA (HLF) from left to right.

FIG. 18 shows that rhPRG4 enhances sorafenib and regorafenib effectiveness in inhibiting cell proliferation, preferentially in high CD44-expressing HCC cells. Cells were tested for growth rate for 72 hours in the presence or absence of sorafenib, regorafenib (2.5 μM), and increasing rhPRG4 concentrations (0 to 100 μg/ml). Drug effectiveness is calculated as % of growth inhibition subtracted by the rhPRG4 inhibitory contribution. T-Test: * p<0.05; ** p<0.01; *** p<0.001. Cell count is shown in the graphs on the top row, while the second row shows plots of cell growth vs. concentration of rhPRG4, and the third row shows plots of % of drug-induced cell growth inhibition vs. concentration of rhPRG4. Control is shown in a dark line, while the results for sorafenib are marked with an “X” and results for regorafenib are marked with a “T.”

FIGS. 19A-B shows CD44 silencing in HCC cells offsets the enhancement of drug effectiveness by lubricin. In FIG. 19A, cells were transduced via lentiviral infection and further selected for stable CD44-silencing. A 72-hour proliferation test was performed and the net effect of lubricin in enhancing the sorafenib or regorafenib inhibitory action was plotted both for Control- and CD44-shRNA cells. The first row shows plots of cell growth vs. concentration of rhPRG4. Control is shown in a dark line, while the results for sorafenib are marked with an “X” and results for regorafenib are marked with a “T.” The second row shows plots of enhancement of drug inhibitory effect as a % vs. concentration of rhPRG4 for control-shRNA (solid line) and CD44-shRNA (dotted line). FIG. 19B, HCC CAFs were stimulated for 48 hours with TGFβ1 and further incubated for 48 hours (without TGFβ1) in serum-free conditions to allow enrichment of the conditioned medium (CM). The CM was then lubricin concentrated and depleted or not. On the left of FIG. 19B is a western blot showing lubricin depletion from CM of TGFβ1-treated CAFs. ID=immunodepleted TGFβ1-treated CAFs-CM using isotype or anti-lubricin antibody; IP=immunoprecipitated lubricin from TGFβ1-treated CAFs-CM using isotype or anti-lubricin antibody. On the right of FIG. 19B, the effect of TGFβ1-treated CAFs-CM lubricin depleted/not depleted on sorafenib and regorafenib inhibitory action against HLF cell proliferation is shown; 20 μg/ml of CM proteins were used in a 72-hour growth test in the presence of 1% FBS. T-Test: * p<0.05; ** p<0.01; *** p<0.001.

FIG. 20 shows CD44 silencing efficiency in HLE and HLF cell lines transduced with lentiviral particles carrying control non-targeting (V), or specific CD44-targeting shRNA sequences.

FIGS. 21A-B shows the level of various sternness markers (0V6, CD133, CD44 and CD90), epithelial markers (AFP, E-Cadh, EpCAM), mesenchymal markers (Vim, N-Cadh, αSMA), and other cancer-related surface proteins (CD13, CD151) detected on the surface of the primary HCC cell line PLC/DC19 isolated from freshly collected surgically resected HCC specimens.

### DETAILED DESCRIPTION

Metastasis is the major cause of cancer related morbidity and mortality. The ability of cancer cells to become invasive and migratory contributes significantly to metastatic growth, which necessitates the identification of novel anti-migratory and anti-invasive therapeutic approaches. Adhesion to surrounding extracellular matrix (ECM) components is critical for cancer cells to invade the ECM and eventually become metastatic. Applicant has now discovered that PRG4 suppresses the ability of the secreted protein transforming growth factor beta (TGFβ) to induce invasive growth of cancer cells and that rhPRG4 suppresses TGFβ-induced invasiveness of cancer cells by inhibiting the downstream hyaluronan (HA)-cell surface cluster of differentiation 44 (CD44) signaling axis. Furthermore, Applicant has discovered that rhPRG4 represses TGFβ-dependent increase in the protein abundance of CD44 and of the enzyme HAS2, which is involved in HA biosynthesis. It is widely accepted that TGFβ has both tumor suppressing and tumor promoting roles in cancer. Applicant's finding that rhPRG4 opposes HAS2 and CD44 induction by TGFβ has implications for downregulating the tumor promoting roles, while maintaining the tumor suppressive aspects of TGFβ actions. These findings support a clinical utility of PRG4 as a therapeutic treatment for cancer.

### PRG4 Protein

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. The molecule has a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT (SEQ ID NO:3). Natural lubricin typically comprises multiple redundant forms of this repeat, which typically includes proline and threonine residues, with at least one threonine being glycosylated in most repeats. The threonine anchored O-linked sugar side chains are critical for lubricin's boundary lubricating function. The side chain moiety typically is a β(1-3) Gal-GalNAc moiety, with the β(1-3) Gal-GalNAc typically capped with sialic acid or N-acetylneuraminic acid. The polypeptide also contains N-linked oligosaccharides. The gene encoding naturally-occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally-located exon 6 contains 940 residues. Exon 6 encodes the repeat rich, O-glycosylated mucin-like domain. This extensive O-linked glycosylated mucin-like domain is necessary for PRG4's boundary lubricating and dis-adhesive properties at various biointerfaces in the body including articular cartilage, tendons, the pericardium, and the ocular surface (Jay et al., Matrix Biol., 2014; 39:17-24).

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum 2009; 60(3):840-7). PRG4 has been shown to exist not only as a monomer but also as a dimer and multimer disulfide-bonded through the conserved cysteine-rich domains at both N- and C-termini. Lubris, LLC has developed a full-length recombinant form of human lubricin. The molecule is expressed using the Selexis Chinese hamster ovary cell line (CHO-M), with a final apparent molecular weight of 450-600 kDa, with polydisperse multimers frequently measuring at 1,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is disclosed in International Patent Application No. PCT/US014/061827.

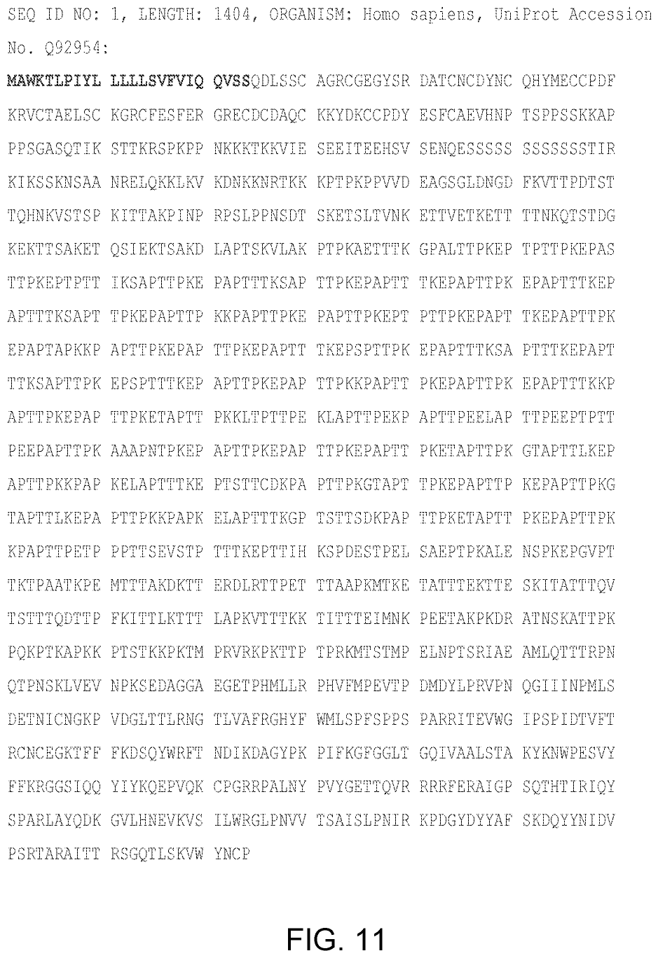

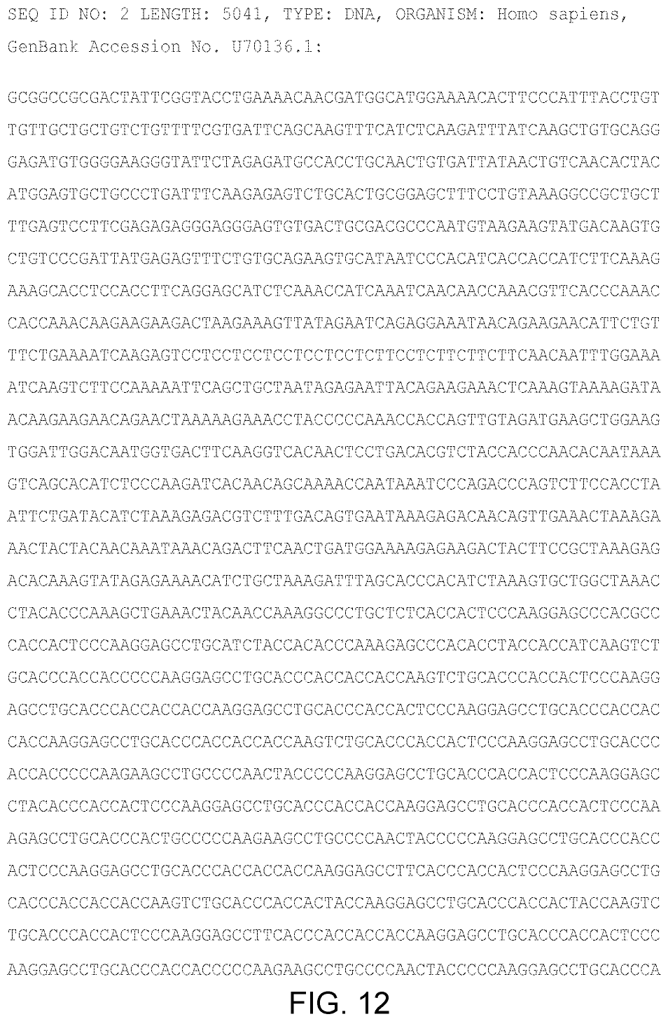

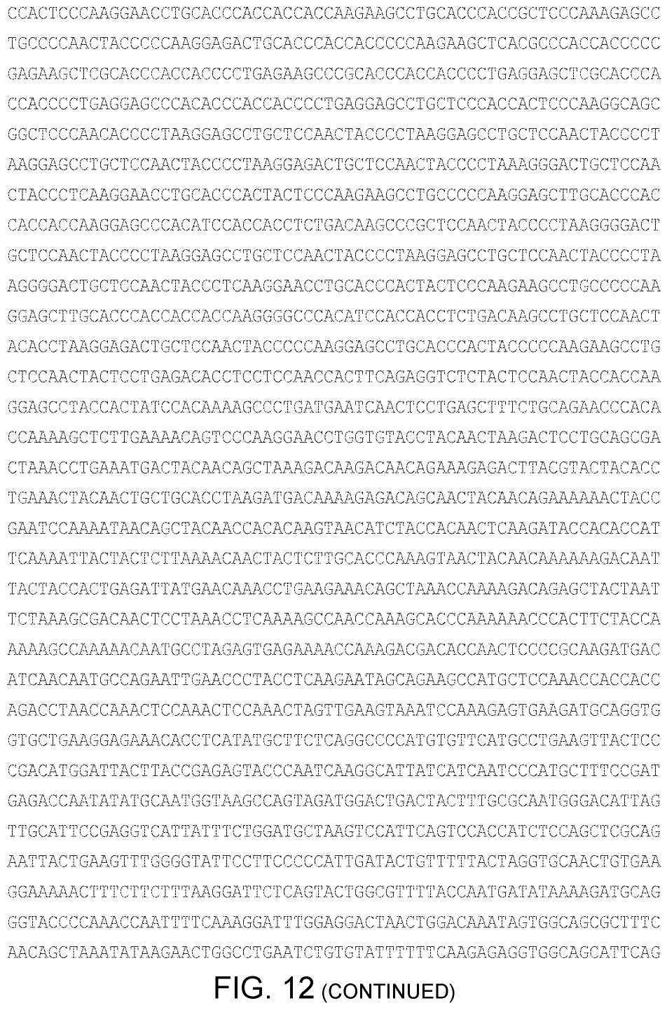

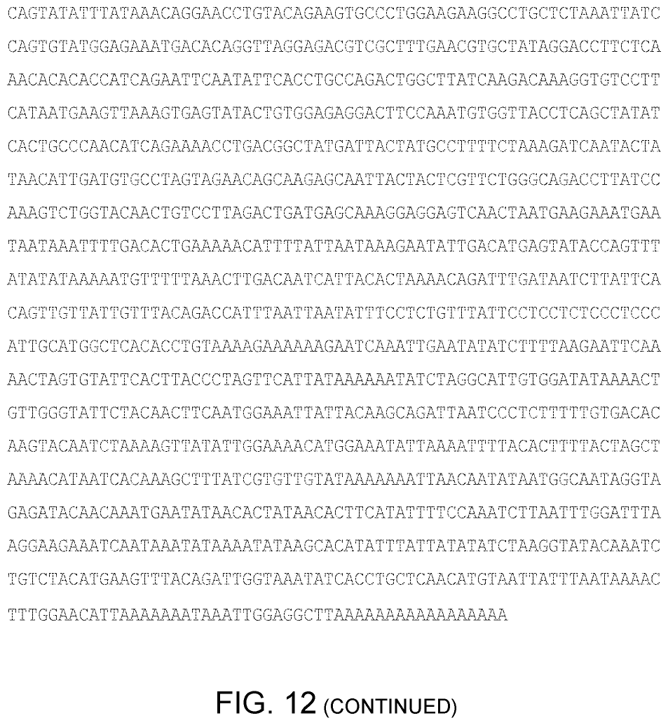

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product, each of which is incorporated herein by reference. Preferred for use in the practice of the invention is full length, glycosylated, recombinant PRG4, or lubricin, expressed from CHO cells. This protein comprises 1,404 amino acids (see FIG. 11; SEQ ID NO:1) including a central exon comprising repeats of the sequence KEPAPTT (SEQ ID NO: 3) variously glycosylated with O-linked β(1-3) Gal-GalNAc oligosaccharides, and including N and C-terminal sequences with homology to vitronectin. The molecule is polydisperse with the glycosylation pattern of individual molecules varying, and can comprise monomeric, dimeric, and multimeric species.

As used herein, the term “PRG4” is used interchangeably with the term “lubricin.” Broadly, these terms refer to any functional isolated or purified native or recombinant PRG4 proteins, homologs, functional fragments, isoforms, and/or mutants thereof. All useful molecules comprise the sequence encoded by exon 6, or homologs or truncated versions thereof, for example, versions with fewer repeats within this central mucin-like KEPAPTT-repeat domain, preferably together with O-linked glycosylation. All useful molecules also comprise at least the biological active portions of the sequences encoded by exons 1-5 and 7-12, i.e., sequences responsible for imparting to the molecule its affinity for ECM and endothelial surfaces. In certain embodiments, a preferred PRG4 protein has an average molar mass of between 50 kDa and 500 kDa, preferably between 224 to 467 kDa, comprising one or more biological active portions of the PRG4 protein, or functional fragments, such as a lubricating fragment, or a homolog thereof. In a more preferred embodiment, a PRG4 protein comprises monomers of average molar mass of between 220 kDa to about 280 kDa. In certain embodiments, PRG4 has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, or at least 99.7% amino acid sequence identity with the amino acid sequence of residues 25-1404 of SEQ ID NO:1. In one embodiment, the sequence identity is at least 98% with the amino acid sequence of residues 25-1404 of SEQ ID NO:1. In one embodiment, the sequence identity of PRG4 is at least 99% with the amino acid sequence of residues 25-1404 of SEQ ID NO:1. In one embodiment, the sequence identity of PRG4 is 99.5% with the amino acid sequence of residues 25-1404 of SEQ ID NO:1. In one embodiment, the sequence identity of PRG4 is 99.6% with the amino acid sequence of residues 25-1404 of SEQ ID NO:1. In one embodiment, the sequence identity of PRG4 is 99.7% with the amino acid sequence of residues 25-1404 of SEQ ID NO:1.

Methods for isolation, purification, and recombinant expression of a proteins such as PRG4 protein are well known in the art. In certain embodiments, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector, and expressed in a host cell for producing recombinant PRG4 protein, and isolated from the cell culture supernatant. A method for production of recombinant human PRG4 is provided in International Patent Application No. PCT/US014/061827.

The function of PRG4 heretofore has been almost entirely associated with reduction of friction and prevention of wear between articulating joints and lubrication of interfacing tissues such as between the surface of the eye and eyelid. The functional importance of PRG4 in joint maintenance has been shown by mutations that cause the camptodactyly-arthropathy-coxa vara-pericarditis (CACP) disease syndrome in humans. CACP is manifest by camptodactyly, noninflammatory arthropathy, and hypertrophic synovitis, with coxa vara deformity, pericarditis, and pleural effusion. Also, in PRG4-null mice, cartilage deterioration and subsequent joint failure were observed. Therefore, PRG4 expression is a necessary component of healthy synovial joints. However, Applicant has now determined that PRG4 can be used to treat or inhibit cancer.

### PRG4 for Use in Treating Cancer

Applicants have determined that PRG4 is able to bind CD44 and is therefore able to down-regulate CD44-mediated receptor signaling. CD44 is a glycoprotein and a major cell surface receptor with various isoforms generated by alternative splicing and glycosylation that plays a major role in inflammation (Cutly et al., J Cell Biol 1992; 116(4):1055-62) and is involved in a variety of cell-cell interactions, tumor metastasis, and lymphocyte activation. CD44 is expressed in a large number of mammalian cell types and its levels of expression vary between cell types and their activation state. Cancerous or neoplastic cells may also express CD44 and the presence of CD44 on such cells is indicative of its involvement in the regulation and metastasis of cancer. In humans, CD44 is encoded by the CD44 gene on chromosome 1. Signaling through CD44 induces T cell proliferation and IL-2 production, dose-response-dependent enhancement of NK cytotoxic activity, and macrophage production of cytokines and chemokines, as well as other functions.

A well-established ligand for CD44 is hyaluronan including high molecular weight (HMW HA), where HMW HA binds to an extracellular motif in CD44 with homology to other HA-binding proteins resulting in subsequent intracellular uptake of HMW HA. (Knudson et al., Matrix Biol 2002; 21(1):15-23; Harada et al., J Biol Chem 2007; 282(8):5597-607; Tibesku et al., Ann Rheum Dis 2006; 65(1):105-8). Low and medium molecular weight hyaluronan (LMW HA and MMW HA) are also ligands for CD44. HA/CD44 interactions are prevalent in a variety of disease states. For example, carcinomas arising from colon epithelia tend to develop in an HA-rich microenvironment, wherein CD44 receptors on epithelial tumor cells activate a tyrosine kinase mediated cell survival pathway, leading to unchecked cell division and proliferation (Misra S et al. Connect Tissue Res. 2008; 49(3):219-24). However, CD44 is also recognized as a marker for cancer stem cells (CSC) and HA is expressed by cancer cells. (Chen et al., J. Hematol. Oncol., 2018; 1:64). For example, expression of CD44 has been noted in head and neck squamous cell carcinoma, breast cancer, pancreatic cancer, gastrointestinal cancers, colorectal adenocarcinoma, prostate cancer, colon cancer, bladder cancer, and leukemia. (Chen et al., J. Hematol. Oncol., 2018; 1: 64). HA binding to CD44 results in activation of cell signaling pathways that induce cell proliferation, increase cell survival, modulate cytoskeletal changes, and enhance cellular motility. (Chen et al., J. Hematol. Oncol., 2018; 1:64).

Other CD44 ligands include extracellular matrix components e.g. collagens, fibronectin and laminin (Naor et al., Adv Cancer Res 1997; 71:241-319; Knudson et al., Cell Mol. Life Sci. 2002; 59:36-44), matrix metalloproteinase-9, the HA-serum-derived hyaluronan associated protein complex (HA-SHAP), hemopexin, EMMPRIN, somatomedin-B, osteopontin, OKT3, or complement related proteins (such as C3a, CD3, CD46).

As demonstrated by the data presented in Example 1 below, the lubricin-CD44 interaction shows that this glycoprotein has functions beyond its boundary lubricating and mechanical properties. In fact, Examples 1A-D show that lubricin acts as a ligand, binding CD44. Accordingly, because lubricin binds CD44, lubricin may be used as a CD44 antagonist to prevent binding to CD44 of ligands, such as hyaluronic acid, and therefore prevent the activation of cell signaling pathways by CD44 activation that induce cell proliferation, increase cell survival, modulate cytoskeletal changes, and enhance cellular motility—activities which are implicated in cancer. Further, the data in Examples 2-7 demonstrate that rhPRG4 suppresses the invasive and migratory ability of cancer cells, specifically showing the direct impact administration of PRG4 can have on curtailing negative behaviors of cancer cells necessary for tumor progression. As demonstrated by the data presented herein, this is achieved at least through rhPRG4's ability to suppress low molecular weight Hyaluronan (LMW HA) signaling via CD44, preventing induction of cancer cell growth.

Evidence suggests that CD44 signaling is involved in the progression of cancer and may also interfere with the effectiveness of certain chemotherapeutic medications. For example, it has been shown that resistance of multiple myeloma to treatment by dexamethasone, a chemotherapeutic, can be caused by CD44 engagement with HA. (Ohwada et al., Eur J Heamatol 2008; 80:245). Accordingly, PRG4 can be used to bind to CD44 on a cancer cell surface to inhibit CD44 signaling involved in cancer cell growth, survivability, progression or metastatic activity. In another embodiment, PRG4 is administered to a patient having cancer or a patient at risk of developing cancer to treat the cancer or slow the growth or progression of a cancer or tumor. According to another embodiment, PRG4 may be administered concurrently with a chemotherapeutic or radiologic treatment for cancer. Accordingly, in one embodiment, PRG4 is administered to a patient having cancer wherein the PRG4 is administered to treat the cancer, or wherein the lubricin is administered in connection with another cancer drug or treatment in order to treat the cancer. In one embodiment, the cancer is in a human subject.

According to one embodiment, the PRG4 is administered to the patient in combination with a chemotherapeutic or radiologic treatment for cancer. For example, in one embodiment, the chemotherapeutic agent is selected from actinomycin, abraxane, altretamine, aranose, azacitidine, azathioprine, bendamustine, bleomycin, bortezomib, busulfan, cabazitaxel, capecitabine, carboplatin, carmofur, carmustine, chlorambucil, chlormethine, chlorozotocin, cisplatin, cladribine, clofarabine, crizotinib, cyclophosphamide, cytarabine, dacarbazine, dactinomycin, dasatinib, daunorubicin, decitabine, docetaxel, doxifluridine, doxorubicin, epirubicin, ertramustine, ethylnitrosourea, erlotinib, etoposide, floxuridine, fludarabine, fluorouracil, fotemustine, gefitinib, gemcitabine, hydroxyurea, hydroxycarbamide, idarubicin, ifosfamide, irinotecan, imatinib, ixabepilone, lapatinib, lomustine, mechlorethamine, melphalan, mercaptopurine, methotrexate, mitomycin, mitoxantrone, nedaplatin, nelarabine, nimustine, nilotinib, N-nitroso-N-methylurea, procarbazine, oxaliplatin, paclitaxel, pemetrexed, pentostatin, ranimustine, raltitrexed, regorafenib, romidepsin, semustine, sorafenib, streptozotocin, tafluposide, tamoxifen, taxotere, tegafur, temozolomide, temsirolimus, teniposide, tioguanine, tofacitinib, opotecan, valrubicin, vemurafenib, vinblastine, vincristine, vincristine, vindesine, vinflunine, vinorelbine, vorinostat, or vismodegib. In one embodiment, the chemotherapeutic agent is regorafenib and/or sorafenib. In one embodiment, the chemotherapeutic agent is regorafenib and/or sorafenib and the cancer is hepatocellular carcinoma.

For example, in one embodiment, the chemotherapeutic agent is an antibody selected from alemtuzumab, bevacizumab, blinatumomab, brentuximab, certolizumab, cetuximab, daratumumab, dinutuximab, ibritumomab, obinutuzumab, ofatumumab, olaratumab, panitumumab, pertuzumab, ramucirumab, rituximab, siltuximab, trastuzumab, rituximab, inotuzumab, gemtuzumab, bevacizumab, camiplimab, or spartalizumab.

According to one embodiment, the PRG4 is administered to the patient in combination with a radiologic treatment for cancer. For example, in one embodiment, the radiologic treatment is external beam radiation therapy, brachytherapy, or stereotactic body radiation therapy (SBRT).

In one embodiment, the administration of PRG4 in combination with a chemotherapeutic or radiologic treatment for cancer enhances the cancer's sensitivity or responsiveness to the chemotherapeutic or radiologic treatment. Accordingly, in one embodiment, administration of PRG4 in combination with a chemotherapeutic or radiologic treatment improves the treatment of the cancer as compared to treatment with the chemotherapeutic or radiologic treatment alone. For example, the combination results increases the speed at which the patient experiences a partial or complete remission of the cancer from the treatment, or the combination increases the likelihood that a patient experiences a partial or complete remission of the cancer from the treatment as compared to treatment with the chemotherapeutic or radiologic treatment alone. In other words, the combination therapy is more effective at treating the cancer than treatment with the chemotherapeutic or radiologic treatment alone.

Co-administration of PRG4 with anti-cancer agents can reduce the toxicity of such agents by allowing them to be administered at lower doses having less toxic effects in patients. Accordingly, in another embodiment, the administration of PRG4 in combination with a chemotherapeutic or radiologic treatment for cancer allows the chemotherapeutic or radiologic treatment to be administered at a dose that is lower than the therapeutic dose for treating the cancer when the chemotherapeutic or radiologic treatment is administered alone. A “therapeutically effective dose” refers to the dose that is demonstrated to show efficacy in treating a specific cancer in a human patient. A “therapeutically effective dose administered alone” refers to the dose of an anti-cancer agent that is demonstrated to show efficacy in treating the specific cancer in a human patient when that anti-cancer agent is the sole anti-cancer agent administered to the patient to treat the specific cancer. The dose may be a daily dose administered over a period of time defining the course of treatment. The dose may be a single dose administered at one time as part of a dosing schedule defining a course of treatment. The therapeutically effective dose may be a dose approved by a government agency. For example, the therapeutically effective dose may be the dose approved by the European Medicines Agency to treat a given cancer. The therapeutically effective dose may be the dose approved by the United States Food and Drug Administration to treat a cancer. In one embodiment, PRG4 is administered with regorafenib, where regorafenib is administered at a dose of less than 160 mg/day. In one embodiment, PRG4 is administered with regorafenib, where regorafenib is administered at a dose of less than 160 mg/day and the cancer is hepatocellular carcinoma. In another embodiment, PRG4 is administered with sorafenib where sorafenib is administered at a dose of less than 800 mg/day or 400 mg/two times per day. In another embodiment, PRG4 is administered with sorafenib where sorafenib is administered at a dose of less than 800 mg/day or 400 mg/two times per day and the cancer is hepatocellular carcinoma. As demonstrated herein, for example, in Example 13, PRG4 enhances the therapeutic effect of anti-cancer treatments, for example, chemotherapeutic agents, when PRG4 is co-administered with such anti-cancer treatments. Consequently, such anti-cancer treatments may be administered at lower doses than otherwise required for therapeutic efficacy.

In another embodiment, PRG4 is administered in combination with an immunotherapy to treat cancer, where the combination treats the cancer. For example, the immunotherapy may be atezolizumab, avelumab, durvalumab, pembrolizumab, nivolumab, cemiplimab, ipilimumab, or another drug that targets PD-L1 or PD-1. Accordingly, in one embodiment, administration of PRG4 in combination with an immunotherapy improves the treatment of the cancer as compared to treatment with the immunotherapy alone. For example, the combination results increases the speed at which the patient experiences a partial or complete remission of the cancer from the treatment, or the combination increases the likelihood that a patient experiences a partial or complete remission of the cancer from the treatment as compared to treatment with the immunotherapy treatment alone. In other words, the combination therapy is more effective at treating the cancer than treatment with the immunotherapy treatment alone.

In another embodiment, the invention provides a method of preventing or inhibiting recurrence of a previously treated cancer in patient. The method involves administering to a patient a therapeutically effective amount of PRG4 to inhibit or prevent recurrence of cancer in the patient where the patient has previously received cancer treatment and experienced remission of the cancer treated, or has had surgery to resect the cancer. In one embodiment, the patient has previously experience a complete remission of the cancer, where as in another embodiment, the patient has previously experienced a partial remission of the cancer. In one embodiment, the prevention or inhibition of recurrence is for at least one year, at least two years, at least three years, at least four years, at least five years, at least six years, at least seven years, at least eight years, at least nine years, at least ten years or more. In another embodiment, the PRG4 is administered at the site of the previously treated cancer annually, bi-annually, quarterly, or biennially after the patient experiences remission or resection of the cancer to prevent or inhibit recurrence of a previously treated cancer in a patient. In another embodiment, the PRG4 is administered systemically to the patient having a previously treated cancer annually, bi-annually, quarterly, or biennially after the patient experiences remission or resection of the cancer to prevent or inhibit recurrence of the previously treated cancer in the patient.

In one embodiment, “treating,” “treat,” or “treatment” of cancer refers to therapeutic intervention that results in reduction in the number and/or size of a tumor, a decrease in the number and/or size of metastases, a decrease in the rate of tumor growth or proliferation, or a decrease in a symptom of the tumor. In some instances, treating cancer according to the methods of the invention results in a patient experiencing complete remission or partial remission of the cancer. In some embodiments, “preventing,” “prevent,” or “prevention” of cancer refers to inhibiting the partial or full development of a cancer. For example, in some embodiments, “preventing” cancer means that a non-cancerous growth or tumor is inhibited from turning into a cancerous tumor, while in some embodiments, prevention of cancer means a cancer is inhibited from recurring, for example, when the cancer has previously been in remission.

Cancers that may be treated with PRG4 according to the method of the invention include adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma (HCC), Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor. In one embodiment, the cancer is breast cancer. In a further embodiment, the cancer is triple-negative breast cancer. In another embodiment, the cancer is hepatocellular carcinoma.

According to methods of the invention, the patient whose cancer is being treated is preferably a human; however, the patient may be any mammal, for example a horse, a cow, a pig, a rat, a mouse, a dog, or a cat.

PRG4 may be co-administered with any of the aforementioned anti-cancer agents. In one embodiment, co-administration means sequential administration of PRG4 and an anti-cancer agent, for example, one after the other, for example on the same day. In other embodiments, co-administration can occur where PRG4 is administered on a different day than the anti-cancer agent(s). In another embodiment, co-administration includes administration of PRG4 and the anti-cancer agent together in one formulation. In another embodiment, co-administration of PRG4 and the anti-cancer agent(s) at the same time as separate formulations. In another embodiment, co-administration refers to PRG4 and the anti-cancer agent(s) being provided at different times during the same course of treatment even if they are not administered at the same moment in time. In one embodiment, PRG4 is administered prior to administration of an anti-cancer agent. In another embodiment, PRG4 is administered after administration of an anti-cancer agent.

### Administration of PRG4

The invention contemplates that PRG4 may be administered to the patient systemically or locally according to the methods of the invention disclosed herein. Local administration may be warranted in cases where the cancer is localized to a specific tissue or organ and accessing the tissue or organ is possible by, for example, injection or local administration. Accordingly, lubricin may be administered locally according to the methods of the invention disclosed herein. For example, lubricin may be locally administered topically or by local injection to the site of a tumor or the site where a tumor has been resected or at a location around or in the vicinity of the tumor site. In one embodiment, the lubricin is administered at the time of a surgical resection of a tumor to the site of the resection after the tumor has been removed. In another embodiment, the lubricin is administered by injection to a non-resectable tumor and/or at or around the site of the non-resectable tumor.

However, lubricin may be administered systemically according to the methods of the invention disclosed herein. Systemic administration is contemplated by some embodiments of the invention, for example, when the cancer is in the blood, lymph, or otherwise cannot be treated by local administration. Systemic administration also may be warranted when the cancer is not localized in one area of the patient but is found throughout the patient or in more than one location in the patient, for example, if the cancer has metastasized. Examples of acceptable modes of systemic administration include enteral delivery, such as oral, rectal, sublingual, sublabial, or buccal delivery or parenteral, such as nasal, by inhalation, intravenous, intramuscular, subcutaneous, intradermal, intraperitoneal or transmucosal delivery. Another acceptable method of systemic administration is by injection, for example, by intravenous administration, by subcutaneous injection, or by intramuscular injection.

In one embodiment of this method, lubricin is provided in an amount that is insufficient to provide boundary lubrication. Applicants have determined that the effects of lubricin on cancer can be achieved at concentrations much lower than what is necessary to achieve boundary lubrication. Accordingly, in one embodiment, lubricin is administered in an amount ranging from 0.1 μg/kg to 4,000 μg/kg. For example, lubricin may be administered in an amount ranging from 0.1 μg/kg to 2000 μg/kg. For example, lubricin may be administered in an amount ranging from 50 μg/kg to 500 μg/kg. For example, lubricin may be administered in an amount ranging from 500 μg/kg to 1000 μg/kg. For example, lubricin may be administered in an amount ranging from 100 μg/kg to 1000 μg/kg. For example, lubricin may be administered in an amount ranging from 2000 μg/kg to 3000 μg/kg. For example, lubricin may be administered in an amount ranging from 2000 μg/kg to 4000 μg/kg. In another embodiment, lubricin is administered in an amount ranging from 0.1 μg/mL to 100 mg/mL, or 25-75 mg/mL, or 30-60 mg/mL. In one embodiment, lubricin is administered at 30 mg/mL and is administered in small volumes of 1 to 100 μL per dose. In another embodiment, lubricin is administered in volumes of 100 μL, to 4 L per dose. In a further embodiment, lubricin is systemically administered to achieve a blood concentration in the range of 10 μg/mL to 100 μg/mL. In yet another embodiment, lubricin is administered in small volumes at the site of a tumor or resected tumor in the amount of 100 μL-5 mL, where the concentration of the lubricin is provided in a range of 0.1 μg/mL to 100 μg/mL or in the range of 10 μg/mL to 1 mg/mL.

The amount of lubricin administered will depend on variables such as the size, type, and location of the cancer and the extent of any metastasis, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Patients may be provided with an induction dose to achieve a certain blood level followed by one or more treatment or maintenance doses. The optimal dose can be determined by routine experimentation.

PRG4 can be administered according to the methods of the invention disclosed herein on a variety of different dosing schedules. For example, in one embodiment, PRG4 is administered once locally at the time of a surgical resection of a cancer. In another embodiment, PRG4 is administered to a patient to treat or prevent cancer every day, every other day, every 3 days, every 4 days, every 5 days, every 6 days, every 7 days, every 14 days, or every 28 days. For example, the patient may receive treatment with PRG4 until the patient experiences a complete or partial remission. In another embodiment, PRG4 is administered to a patient having a tumor that is not yet cancerous to prevent the tumor from becoming cancerous on a yearly basis, on a biannual basis, on a quarterly basis or on a monthly basis. In another embodiment, the PRG4 is administered to a patient on the same day the patient receives another anti-cancer treatment such as chemotherapy or radiation and is administered each time the patient receives that other anti-cancer treatment. In another embodiment, the PRG4 is administered to a patient on the day before the patient receives another anti-cancer treatment such as chemotherapy or radiation and is administered the day before each time the patient receives that other anti-cancer treatment. In another embodiment, the PRG4 is administered to a patient on the day after the patient receives another anti-cancer treatment such as chemotherapy or radiation and is administered the day after each time the patient receives that other anti-cancer treatment.

### Formulations of PRG4

For administration, lubricin is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. Carriers may also include biomaterials such as a matrices, hydrogels, polymers, tissue scaffolds, and resorbable carrier materials including collagen sponges. Exosomes and the like may also be used as carriers. The use of such media and agents for pharmaceutically active substances is known in the art. Useful formulations can be prepared by methods well known in the pharmaceutical art. For example, see Remington's Pharmaceutical Sciences, 18^{th} ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. Lubricin for administration can be presented in a dosage unit form and can be prepared by any suitable method and should be formulated to be compatible with its intended route of administration.

### EXAMPLES

### Example 1: Experimental Evidence that PRG4 Antagonizes CD44

To evaluate the interaction between human PRG4 and the CD44 receptor and the consequence of this interaction, the following experiments were performed.

rhPRG4 binding to CD44 and competition with high molecular weight hyaluronic acid (HMW HA) was evaluated using a direct enzyme linked immunosorbent assay (ELISA) and surface plasmon resonance. Sialidase-A and O-glycosidase digestion of rhPRG4 was performed and CD44 binding was evaluated using ELISA. Rheumatoid arthritis fibroblast-like synoviocytes (RA-FLS) were stimulated with interleukin-1 beta (IL-1(3) or tumor necrosis factor alpha (TNF-α) for 48 hours in the presence or absence of rhPRG4 or HMW HA at 20, 40 and 80 μg/mL and cell proliferation was measured. CD44 contribution was assessed by co-incubation with an anti-CD44 antibody (IM7). The anti-proliferative effect of rhPRG4 was investigated following treatment of Prg4−/− synoviocytes with IL-1β or TNF-α in the presence or absence of IM7.

Variables were initially tested for normality and equal variances. Variables that satisfied both assumptions were tested for statistical significance using Student's t-test or analysis of variance (ANOVA) with Tukey's post-hoc test for two group and more than two group comparisons, respectively. Variables that did not satisfy the normality assumption were tested using Mann-Whitney U test or ANOVA on the ranks. The level of statistical significance was set at α=0.05. Data is graphically represented as the average±standard deviation.

1A. Binding of rhPRG4, High-Molecular Weight HA, Medium-Molecular Weight HA and Vitronectin to CD44 Using a Direct ELISA

High-binding microtiter plates (Corning, Sigma Aldrich, USA) were coated with rhPRG4 (Mᵣ≅240 KDa), high molecular weight HA (HMW HA; Mᵣ≅1,500 KDa) (R & D System, USA), medium molecular weight HA (MMW HA; Mᵣ≅′300KDa) (R & D System) and vitronectin KDa) (Sigma Aldrich) at 400 μg/mL in PBS buffer (100 μL per well) overnight at 4° C. rhPRG4 is a full-length product produced by CHO-M cells (Lubris, Framingham, Mass., USA). Following washing with PBS+0.1% Tween 20, wells were blocked with 2% bovine serum albumin (BSA; 300 μL per well) for at least 2 hours at room temperature. CD44-IgG₁Fc (R & D systems) or IgG₁Fc (R & D systems), each at 1 μg/mL (100 μl per well), were added to the plate and incubated for 60 min at room temperature. Following washing with PBS+0.1% tween 20, anti-IgG₁Fc-HRP (Sigma Aldrich) was added at 1:10,000 dilution (100 μL per well) and incubated for 60 min at room temp. Following washing with PBS+0.1% tween 20, the assay was developed using 1-step Turbo TMB ELISA reagent (ThermoScientific, USA) and absorbance was measured at 450 nm. The data represents an average of 4 independent assays, each with triplicate wells per group.

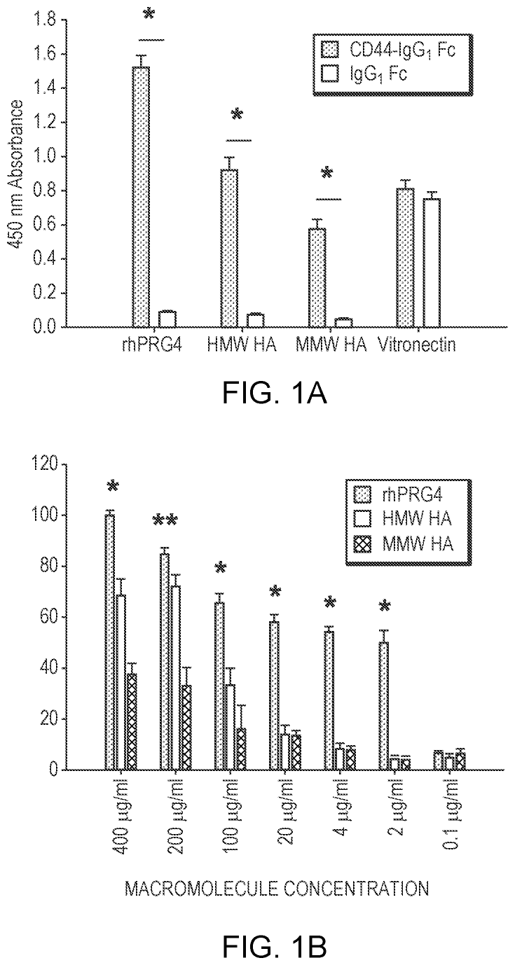

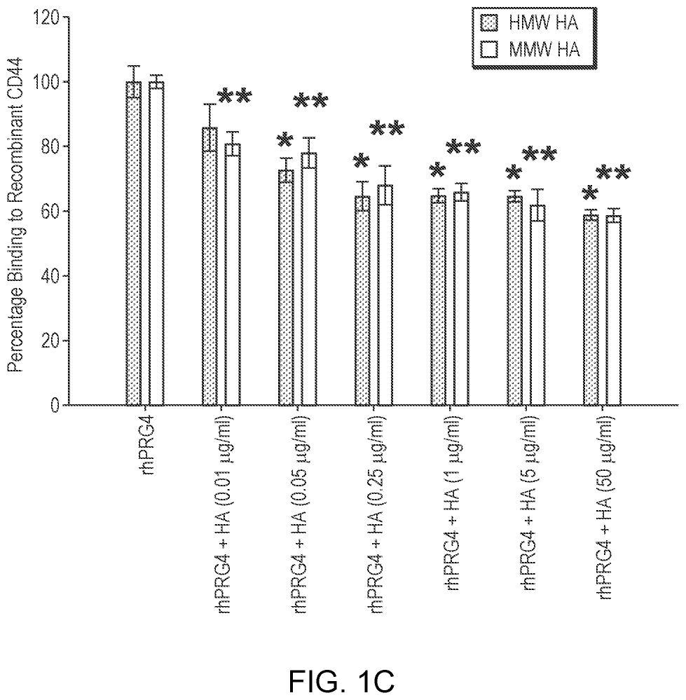

Binding of rhPRG4, HMW HA, MMW HA and vitronectin to CD44-IgG₁Fc fusion protein and IgG₁Fc is presented in FIG. 1A. The 450 nm absorbance in the CD44-IgG₁Fc group was significantly higher (p<0.001) than the absorbance in the IgG₁Fc group for rhPRG4, HMW HA and MMW HA-coated wells. In contrast, there was no significant difference between CD44-IgG₁Fc and IgG₁Fc in the vitronectin-coated wells.

These data show that rhPRG4 binds CD44 and interferes with HMW HA CD44 binding. rhPRG4, HMW HA and MMW HA specifically bind to chimeric CD44 with extremely low non-specific binding. In contrast, vitronectin that shares significant sequence homology with lubricin does not show any specificity towards CD44 binding. Because rhPRG4 binds CD44, it may function as an antagonist of CD44, thereby interfering with CD44 pro-inflammatory signaling.

1B. Concentration-Dependent Binding of rhPRG4, HMW HA, MMW HA to CD44 and Competition Between rhPRG4 and HA on Binding to CD44 Using a Direct ELISA

The concentration-dependent binding of rhPRG4, HMW HA and MMW HA to CD44 was performed by coating microtiter plates with 400, 200, 100, 20, 4, 2 and 0.1 μg/mL of the macromolecules. The assay was performed as described above. The absorbance values in the IgG₁Fc wells were subtracted from the absorbance values in the CD44 IgG₁Fc wells and the corrected CD44 IgG₁Fc absorbance values were normalized to those of the 400 μg/mL rhPRG4 group and data was expressed as percentage binding to CD44. The concentration-dependent binding of rhPRG4, HMW HA and MMW HA to recombinant CD44 is depicted in FIG. 1B. The percentage recombinant CD44 binding was significantly higher (p<0.001) in the rhPRG4-coated wells compared to the HMW HA or MMW HA-coated wells for the 400, 100, 20, 4 and 2 μg/mL concentrations. Additionally, the percentage recombinant CD44 binding was significantly higher (p<0.001) in the rhPRG4-coated wells compared to the MMW HA coated wells for the 200 μg/mL concentration. There were no significant differences in percentage CD44 binding between the rhPRG4, HMW HA and MMW HA-coated wells at the 0.1 μg/mL concentration. The data represents an average of 4 independent assays, each with triplicate wells per group.

To evaluate the competition between rhPRG4 and either HMW HA or MMW HA on binding to CD44, microtiter plates were coated with either CD44 IgG₁Fc or IgG₁Fc at 1 μg/mL (100 μL per well) overnight at 4° C. Subsequently, wells were washed with PBS+0.1% tween 20 and wells were blocked using 2% BSA (300 μL per well) for at least 2 hours at room temperature. Either rhPRG4 at 5 μg/mL or a combination of rhPRG4 (5 μg/mL) and HMW HA or MMW HA at 0.01, 0.05, 0.25, 1, 5 or 50 μg/mL were added to the wells (100 μL per well) and incubated at room temperature for 60 min. Following washing with PBS+0.1% tween 20, lubricin-specific monoclonal antibody (Mab 9G3) was added at 1:1,000 (100 μL per well) and incubated for 60 min at room temp. Following washing with PBS+0.1% tween 20, goat anti-mouse IgG-HRP (Thermo Scientific) at 1:1,000 dilution was added (100 μL per well) and incubated for 60 min at room temp. The assay was developed as described above. The absorbance values in the IgG₁Fc wells were subtracted from the absorbance values in the CD44-IgG₁Fc wells and the corrected absorbance values in the rhPRG4+HA groups were normalized to the absorbance values of the rhPRG4 group and data was expressed as percentage binding to CD44. The data represents an average of 4 independent assays, each with triplicate wells per group.

The competition between rhPRG4 and HMW HA or MMW HA in binding to recombinant CD44 is presented in FIG. 1C. HMW HA or MMW HA at 0.05, 0.25, 1, 5 and 25 μg/mL significantly reduced rhPRG4's binding to CD44 (p<0.05).

These data demonstrate that rhPRG4 binds to CD44 in a concentration-dependent manner with comparable affinity to HMW HA. Furthermore, rhPRG4 competes with HMW HA in binding to CD44. The presence of an excess of HMW or MMW HA reduced rhPRG4 binding to CD44 only by approximately 50%. These data suggest that rhPRG4 is an antagonist of CD44; accordingly, it has the potential to interfere with CD44 signaling.

1C. Concentration-Dependent Binding of rhPRG4 to CD44 and Competition Between rhPRG4 and HMW HA Using Surface Plasmon Resonance

Binding of rhPRG4 to CD44-IgG1Fc was investigated using surface plasmon resonance (Biacore T100, GE Healthcare Lifesciences, NJ, USA). See FIG. 1C. Series S chips were functionalized using the human antibody capture kit (GE Life Sciences) and either CD44-IgG₁Fc or IgG₁FC was allowed to bind to the surface of the functionalized chips in flow cell 1 (Fc₁) and flow cell 2 (Fc₂), respectively. rhPRG4 was injected at 30 μL/min for 8 min at concentrations of 300, 250, 200, 150, 100 and 50 μg/mL followed by a 10 min dissociation using 0.1M HEPES, 1.5M NaCl, 30 mM EDTA, and 0.5% P20 (GE Life Sciences). The surface of the chip was regenerated at the end of each cycle with 1 min pulse of 3M MgCl₂. Each analyte concentration was injected in duplicate. The resulting curves were double referenced (i.e. Fc₂-Fc₁, followed by subtraction of the 0 μg/mL curve). The binding kinetics and binding affinity were determined by BiaEvaluation software, using 1:1 binding/conformational change model or by steady-state equilibrium, respectively. To study the competition between rhPRG4 and HMW HA in binding to CD44, rhPRG4 was injected at concentrations ranging between 0 and 300 μg/mL as described above. Following the end of dissociation phase, HMW HA was injected at 50 μg/mL (30 μL per min) for 1 min. The double-referenced binding signals of rhPRG4 (at various concentrations) to CD44 were then plotted against the binding signals generated by HMW HA binding to CD44 following rhPRG4 injections.

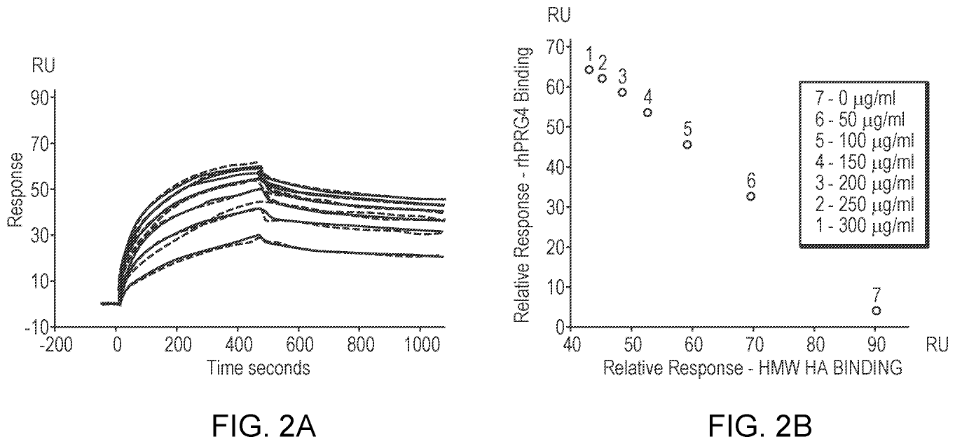

The binding of rhPRG4 to recombinant CD44 was confirmed using surface plasmon resonance. rhPRG4 displayed a concentration-dependent association with, and dissociation from immobilized CD44-IgG₁Fc (FIG. 2A), with an apparent K_{d}≅38 nM based on a rhPRG4 molecular weight of 240 KDa. rhPRG4 interfered with binding of HMW HA to recombinant CD44 as shown by an inverse relationship between the HMW HA binding signal intensity (x-axis) and the rhPRG4 binding signal intensity (y-axis) (FIG. 2B).

These data demonstrate that rhPRG4 binds to CD44 in a concentration-dependent manner with comparable affinity to HMW HA. Further, as demonstrated in Example 1B and 1C, the presence of rhPRG4 bound to CD44 prevented HMW HA from binding to CD44 in a concentration-dependent manner and may indicate that rhPRG4 and HMW HA share a common binding site on the receptor. In the joint environment where HA SF concentration is roughly 10 times higher than that of lubricin, and based on the competitive binding data shown herein, it is expected that lubricin will be able to bind to CD44 on surface of synoviocytes and chondrocytes and exert a CD44-mediated biological function in the presence of HA, thereby providing a joint homeostatic role by interfering with mediators that otherwise promote inflammation.

1D. Impact of Removal of Mucin-Domain Glycosylations on Binding of rhPRG4 to CD44

Lubricin's boundary lubricating ability is mediated by the O-linked (β1-3) Gal-GalNAc oligosaccharides (Jay et al., Glucoconj J 2001; 18(10):807-15). A combination of neuraminidase and beta 1, 3, 6 galactosidase digestions reduced lubricin's boundary lubricating ability by 50% (Jay et al., Glucoconj J 2001; 18(10):807-15). Lubricin isolated from RA SF samples contains increased core 1 glycosylation structures and displays the sulfated epitope that is proposed to be part of the L-selectin ligand (Estrella et al., Biochem J 2010; 429(2):359-67). Additionally, lubricin from RA SF binds L-selectin in a glycosylation-dependent manner and coats polymorphonuclear granulocytes recruited to inflamed synovia and SF of patients with RA (Jin et al. J Biol Chem 2012; 287(43):35922-33).

rhPRG4 was digested using sialidase A (Prozyme, USA), O-glycosidase (New England Biolabs, USA) or a combination of sialidase A and O-glycosidase for 16 hours at 37° C. In the sialidase A digestion, 12 μL of the enzyme (1U/200 μL) was added to rhPRG4 in a total reaction volume of 180 μL and a rhPRG4 final concentration of 300 μg/mL. In the O-glycosidase digestion, 4.8 μL of the enzyme (40million units/mL) was added to rhPRG4 in a total reaction volume of 180 μL and a rhPRG4 final concentration of 300 μg/mL under non-denaturing conditions. In the sialidase-A and O-glycosidase digestion, the enzymes were used in volumes identical to the ones stated above and incubated with rhPRG4 in a total reaction volume and final rhPRG4 concentration as stated above. The effect of sialidase-A and O-glycosidase digestions on rhPRG4 apparent molecular weight was determined by SDS-PAGE using 4-12% Bis-Tris gel (NuPage, life technologies, USA). A total of 20 μL of rhPRG4 or enzyme-digested rhPRG4 was run under reducing conditions (200 mV for 60 min) followed by staining using Gelcode Blue Stain (Thermo Scientific, USA). Binding of enzymatically digested rhPRG4 to CD44 was compared to undigested rhPRG4 using the direct ELISA approach described above and using an rhPRG4 coating concentration of 30 μg/mL. Data represents the average of 4 independent experiments, each with triplicate wells per group.

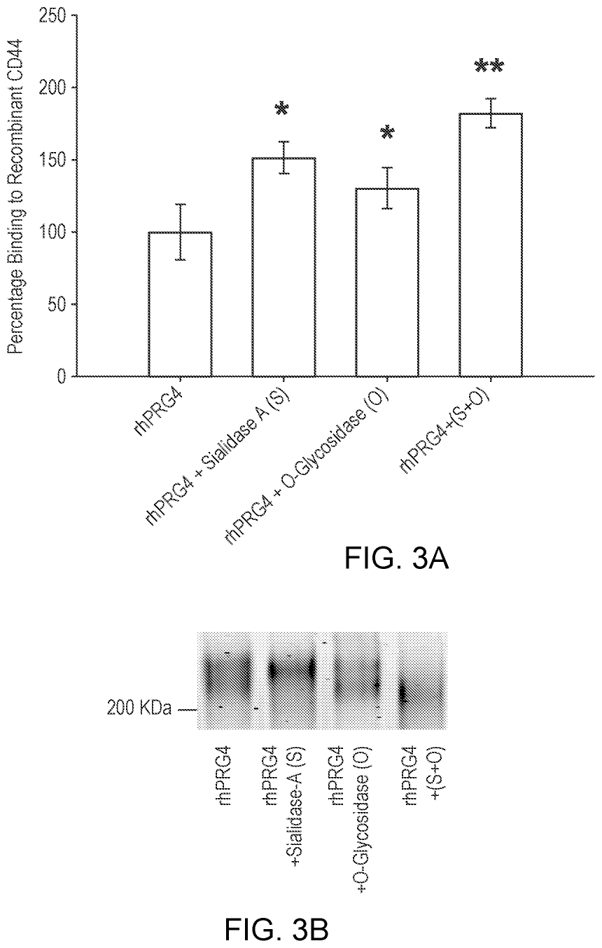

Sialidase-A digestion resulted in a significant increase (p<0.001) in the percentage binding of rhPRG4 to CD44 compared to untreated control as shown in FIG. 3A. Similarly, O-glycosidase digestion resulted in a significant increase (p=0.008) in the percentage binding of rhPRG4 to CD44 compared to untreated control. There was no significant difference in percentage CD44 binding between the sialidase-A digested and the O-glycosidase codigested rhPRG4 (p=0.105). The percentage binding to CD44 in the sialidase-A and O-glycosidase-digested rhPRG4 was significantly higher than sialidase-A digested rhPRG4 (p=0.007), O-glycosidase digested rhPRG4 (p<0.001) and untreated control (p<0.001). Digestion of rhPRG4 with sialidase-A and O-glycosidase resulted in reducing the apparent molecular weight of rhPRG4 to approximately 200 KDa (FIG. 3B).

Removal of sialic acid and O-glycosylations significantly increased CD44 binding by rhPRG4 (p<0.001). Sialidase-A and O-glycosidase treatments individually resulted in enhancing rhPRG4's binding to CD44 receptor. Cumulative sialidase-A and 0-glycosidase digestions resulted in even more significant binding to CD44 by rhPRG4 compared to individual enzyme digestions. Sialidase-A cleaves branched and unbranched terminal sialic acid residues from glycoproteins, while O-glycosidase catalyzes the removal of cores 1 and 2 from glycoproteins. The enhancement in CD44 binding indicates that neither the core 1 glycosylation nor the sialic acid terminal residues are required in rhPRG4 binding to CD44. Accordingly, the level of sialylation and core 1 glycosylations on rhPRG4 protein core are not essential to the PRG4's ability to bind CD44. In contrast, removal of these residues may lead to a conformational change in the rhPRG4 semi-rigid rod shaped structure that results in enhanced interaction with CD44.

### Example 2. Materials and Methods for Examples 3-9

(1) Plasmids

The pU6/CD44 RNA interference-1/CMV-enhanced green fluorescent protein (EGFP) and pU6/CD44 RNA interference-2/CMV-EGFP expression vectors, abbreviated as CD44i-1 and CD44i-2, containing the CD44 sequences 5′GAGCAGCACTTCAGGAGGTTA3′ and 5′CTCCATCTGTGCAGCAAACAA3′, respectively, were generated as described Sarker et al. (J. Biol. Chem. 2005; 280(13):13037-46). In each of the plasmids CD44i-1 and CD44i-2, the mouse U6 small RNA promoter and CMV promoter induce the expression, respectively, of a human CD44 mRNA-targeting short hairpin (sh) RNAs (shRNAs) and EGFP. The pU6/CMV/EGFP control RNAi vector has been described. (Sarker et al., J. Biol. Chem. 2005; 280(13):13037-46). Expression of the EGFP protein by fluorescence microscopy indicated vector control or CD44i-1/2 transfected cells. The pCMVSB/CD44/FLAG expression vector was generated by a T4 DNA ligase (New England BioLabs, USA)-based ligation of a C-terminally FLAG tagged human open reading CD44 cDNA (CD44/FLAG) into the pCMV5B vector (Kaysak et al., Molecular Cell. 2000; 6(6):1365-75). CD44/FLAG DNA was generated by a polymerase chain reaction (PCR) using Pwo polymerase (Roche Diagnostics, USA), polyA-enriched cDNA as template, and 5′ CCCACGCGTACCATGGACAAGTTTTGGTGGC 3′, and 5′ CCCTCTAGATTACTTGTCA TCGTCGTCCTTGTAGTCCAGTCGACCCACCCCAATCTTCATGTCC 3′, respectively, as forward and reverse primers. The poly A cDNA was generated by subjecting MDA-MB-231 cell TRIzol-(Ambion Life technologies, Canada) extracted mRNA to reverse transcription (RT)-PCR reaction using the SuperScript II transcriptase (Invitrogen, Canada) and the primer oligo-(dT)12-18 (Amersham Biosciences, UK). CD44i-1/2, and CD44/FLAG plasmids were verified by DNA sequence analyses (University of Calgary Core Sequencing Facility).

(2) Cell Lines and Transfections

MDA-MB-231 cells were purchased from American Type Culture Collection (ATCC, USA), and cultured in Dulbecco's modified Eagle medium (DMEM; Invitrogen, Canada) supplemented with 10% fetal bovine serum (FBS; Thermo Fisher, Canada). The cells were kept in 37° C. in a 5% CO₂ humidified cell incubator, and were routinely passaged every 3-4 days. The MDA-MB-231 cells were transfected using Lipofectamine 3000 reagents (Invitrogen, Canada).

(3) Reagents

The following reagents were used to treat cells in different assays in the study: recombinant human proteoglycan 4 (rhPRG4; Gift from Lμbris Biopharma, USA; stock 1 to 2.5 mg/mL in phosphate-buffered saline (PBS)) (Abubacker et al., Ann Biomed Eng. 2016; 44(4):1128-37; Samsom et al., Exp Eye Res. 2014; 127:14-9), recombinant human mature transforming growth factor beta (TGFβ) (R&D systems, USA; stock 10 μM), Low molecular weight hyaluronic acid (LMWHA; Sodium Hyaluronate; HASK; Lifecore Biomedical, USA; stock 10 mg/mL in PBS), Kinase inhibitor (KI; SB431542; Millipore-Sigma, Canada; stock 10 mM) (Halder et al., Neoplasia. 2005; 7(5):509-21), CD44 neutralizing antibody (Thermo Fisher Scientific, Canada) (Kariya et al. BBA Clinical. 2015; 3:126-34), Anti-PRG4 mAb 4D6 (Gift from Dr. Phillip Messersmith, University of California, Berkeley) (Abubacker et al., Connective Tissue Research. 2016; 57(2):113-23; Chawla et al., Acta biomaterialia. 2010; 6(9):3388-94), Mouse Immunoglobulin G (Mouse IgG; Santa Cruz, USA), 4-methylumbelliferone (4-MU; Millipore-Sigma, Canada; stock 10 mM in DMSO).

(4) Three-Dimensional Cultures

For three-dimensional culture assays, 50 μl of ice-cooled 33% growth factor-reduced Matrigel (Corning Incorporated, USA) in complete growth medium, consisting of DMEM containing 10% FBS, penicillin, streptomycin and amphotericin B (Invitrogen, Canada), was added per well of a 96-well flat bottom, ultra-low attachment plate (BD Biosciences, Canada) followed by storing the plate for 1 hour in a 5% CO₂ humidified incubator at 37° C. to allow formation of a 1 mm-thick Matrigel bed in the well. Next, approximately 400 isolated MDA-MB-231 cells, suspended in 50 μl ice-cooled 50% Matrigel in complete growth medium, were layered on top of the Matrigel bed per well of the 96-well plate and incubated inside 5% CO₂ humidified incubator at 37° C. One hour post addition and 37° C. incubation of Matrigel-cells suspension, 50 μl of complete growth medium was added to cover the solidified Matrigel-cells mixture. The following day and, every third day, the three-dimensional cultures received 50 μl of fresh complete growth medium without or with specified reagents as listed in the figure descriptions. On Day 8 of culture, differential interference contrast (DIC) images were captured of 8 representative organoids from each well, using light microscopy at 30× objective (Olympus IX70, Canada). Observation of the Matrigel-embedded cultures by light microscopy at different days of three-dimensional cultures, indicated that isolated cells, after undergoing successive divisions, each gave rise to a multicellular structure, here referred to as an organoid. In the control setting, the majority of the MDA-MB-231-cell-derived organoids displayed smooth-surfaced spherical phenotype, which can change depending on the incubation conditions of the 3D cultures. Number of smooth surfaced organoids without any protrusions, designated as “spherical” were counted out of eight representative images in each well and then percentage of spherical organoids in each condition were plotted in bar graphs. Each experiment was performed at least three independent times for statistical analyses.

(5) Immunocytochemistry and Fluorescence-Cell Based Analyses

For immunofluorescence analysis of 3D-organoids, 600 MDA-MB-231 cells were grown within 80 μL of 50% Matrigel on top of 80 μL of 33% Matrigel coat in each well of ultra-low attachment 8-well chamber slides (Millipore-Sigma, USA). After DIC imaging on Day 8, the live multicellular structures were fixed with 4% formaldehyde, followed by permeabilization using 0.5% ice-cold Triton X-100 solution and blocking using 10% bovine serum albumin (BSA) in phosphate-buffered saline (PBS). For immunofluorescence analysis of monolayer cell culture, cells were seeded onto wells of a Falcon 8-well chamber culture slides (Corning Incorporated, USA), followed by fixing and permeabilization as described for the 3D cultures, and blocking with 5% BSA and 5% calf serum in PBS. Laminin and CD44 in cells were visualized by subjecting the three-dimensional or monolayer cultures to indirect immunofluorescence using a rat anti-laminin antibody (Abcam, Canada) and rat anti-CD44 antibody (Thermo Fisher, Canada) as the primary antibody, respectively, and Alexa 647-conjugated anti-rat IgG (ThermoFisher, Canada) as the secondary antibody. Actin in cells was visualized by incubating the fixed cultures with tetramethylrhodamine isothiocyanate (TRITC)-conjugated phalloidin (Millipore-Sigma, Canada). The DNA binding dye bisbenzimide (Hoechst 33342; Invitrogen, Canada) was used to detect nuclei. For CD44 knockdown analysis, the vector or CD44 shRNA transfected cells were identified by GFP signal. Immunofluorescence images were captured using an epifluorescence microscope with a 40× objective lens (Olympus Bx WI Confocal Microscope, Canada). Exposure times for laminin, actin, nuclei and GFP-specific signals were kept constant in each experiment. For each condition, 3 colonies/fields per experiment were captured, which were chosen as representative of the stained cells within each slide per experiments. Each experiment was repeated two independent times.

(6) In Vitro Trans-Well Invasion Assays

Overnight 0.2% FBS-containing DMEM incubated, i.e. serum-starved, MDA-MB-231 cells were used for the transwell invasion using polycarbonate filters (24-well insert, pore size 8 μm; BD Biosciences, Canada). Prior to addition of cells, each insert was placed within a well of a 24-well tissue culture plate and equilibrated with 0.5 mL serum-free DMEM, added both to the upper and lower chambers at 37° C. for 2 h. The equilibration media was then gently removed and upper chamber surface of the insert was coated with 50 μL of 3% Matrigel and allowed to solidify at 37° C. for 1 h. 1×10⁵ serum-starved MDA-MB-231 cells were resuspended in 0.5 mL of serum-free DMEM and added to the upper Matrigel-coated chamber. 500 μL complete growth medium alone or containing specific reagents, as described in the respective figure descriptions, was added to the lower chambers. Cells were allowed to invade the matrix for 12 h at 37° C. after which non-adherent cells were removed by PBS washing of cell layers on the upper chamber three times. During the second wash a cotton tip applicator was used to gently scrape away the adherent cells on the upper surface of the membrane. Invading cells were fixed by immersing the transwell inserts in 100% methanol for 10 minutes at −20° C., followed by staining with 0.5% crystal violet dye (EMD Millipore, Canada) for 1 h at room temperature. Eight randomly chosen fields of each stained membrane were imaged at 10× objective of a DIC microscope (Olympus IX70) coupled to a digital camera. Cell numbers were obtained by counting the number of crystal violet stained cells in each field using a handheld counter. The average cell numbers for all images per treatment conditions were calculated and used for further statistical analysis. Each experiment was performed at least three independent times.

(7) In Vitro Scratch Assays

5×10⁵ MDA-MB-231 cells were seeded in each well of a 12-well tissue culture plate and grown overnight to near confluency in complete growth medium and then overnight serum starved by incubating with 0.2% FBS-containing DMEM medium in a 5% CO2 humidified incubator at 37° C. incubator. Using a 200 μL pipette tip, a scratch was introduced along the midline of the overnight serum starved cell monolayers, followed by a PBS wash to remove floating cells, and incubating the cells with 0.2% FBS-containing medium without or with TGFβ, alone or together with KI or rhPRG4 for 36 h in a 5% CO₂ humidified incubator at 37° C. incubator. Scratch closure in each well was followed by imaging the scratch and surrounding cells in each well at 3× objective of a DIC microscope (Olympus IX70) coupled to a digital camera at time 0 h and 36 h after initiating the scratch. Five images were captured along the vertical axis of the scratch for each experimental condition. The width of each scratch was measured at three different positions per image for a total of 15 measurements using ImageJ (National Institutes of Health, USA), and then averaged per experimental condition. The width average at 36 h was subtracted from the width average at 0 h and expressed relative to that at 0 h width for each experimental condition to obtain scratch closure, and expressed as percent scratch closure.

(8) Cell Extract Preparation, Immunoprecipitation and Immunoblotting

Cells were washed with PBS to remove all growth media. Appropriate volume of TNTE lysis buffer (50 mM Tris, 150 mM NaCl, 1 mM EDTA, 0.5% [v/v] Triton-X-100) containing protease and phosphatase inhibitors, was added to the cell monolayer and incubated at 4° C. under vigorous shaking for 20 minutes. Lysates were then collected in microcentrifuge tubes and centrifuged at 13,000 g for 10 minutes at 4° C. 5 μL of the lysate were subjected to protein concentration determination using Bradford-based protein assays (Bio-Rad Laboratories, Canada). Lysates were boiled for 3 minutes at 95° C. in dithiothreitol (DTT)-containing Laemmli sample buffer. For immunoprecipitation analyses, lysates were incubated with appropriate antibodies at 4° C. with gentle rocking for 3 h after which immunocomplexes were incubated with Protein G-conjugated agarose beads (UBPBio, USA) at 4° C. with gentle rocking for 3 h. Finally, the beads were washed with TNTE wash buffer (0.1% [v/v] Triton-X-100) and boiled for 5 minutes at 95° C. in dithiothreitol (DTT) containing Laemmli sample buffer. Immunoprecipitates and input lysates were then resolved by sodium dodecyl sulfate-polyacrylamide electrophoresis (SDS-PAGE) and transferred onto a nitrocellulose membrane (Bio-Rad Laboratories, Canada). The blots were blocked using 5% skim milk followed by overnight incubation with mouse anti-actin (Santa Cruz, USA), rabbit anti-pSmad2 (Abcam, Canada), Mouse anti-Smad2/3 (Millipore-Sigma, Canada), rat anti-CD44 (Thermo Fisher, Canada), Mouse anti-HAS2 (Santa Cruz, USA) or Mouse anti-FLAG (Millipore-Sigma, Canada) as the primary antibody at 4° C. Then HRP-conjugated goat anti-mouse or anti-rabbit IgG (Jackson Laboratories, USA) or anti-rat IgG (Millipore-Sigma, Canada) was added to the blots for 1 h at room temperature, followed by enhanced chemiluminescence (Millipore-Sigma, Canada) and signal detection using a VersaDoc 5000 Imager (Bio-Rad Laboratories). Densitometric analyses were performed using Quantity One software (Bio-Rad Laboratories, Canada).

(9) Reporter Assays

MDA-MB-231 cells were seeded in 24-well plates at approximately 6×10⁴ cells/well one day prior to transfections. Cells were co-transfected with the PAI1-promoter-driven firefly luciferase Reporter (3TP-Lux) and the CMV-Renilla luciferase control reporter constructs. 18 h post transfection, cells were serum-starved (0.2% FBS containing DMEM) for 4 h and then incubated in fresh low-serum (0.2% FBS containing DMEM) containing media in the absence or presence of 100 pM TGFβ, 100 μg/mL rhPRG4 alone or together and left overnight. Lysates were prepared and analyzed for luciferase activity using a commercially available dual luciferase assay kit (Promega, Canada). Arbitrary luciferase activity (relative light units) values were normalized to Renilla luciferase activity to account for variations in transfection efficiency. For each transfection, percent increase of PAI1 promoter driven luciferase reporter gene expression was also determined and expressed relative to luciferase activity of the respective basal condition lysates. Each experimental condition was carried out in triplicate.

(10) Statistical Analyses

Biochemical and organoid related data were subjected to statistical analysis by Student's t-test or One-way analysis of variance (ANOVA) followed by Tukey-Kramer or Student-Newman-Keuls post hoc test using InStat (Graphpad, USA). Values of P<0.05 were considered statistically significant. Data were presented graphically as mean±SEM from experiments that were repeated at least three independent times.

### Example 2: rhPRG4 Promotes Anti-Invasive Growth of 3D-Breast Cancer Cell-Derived Organoids

To evaluate the effect of rhPRG4 on the invasiveness of breast cancer cells, we employed a three-dimensional (3D) cell culture system where cells are allowed to grow and interact with ECM components, such as Matrigel which resembles the ECM that is found in vivo (27). Cellular responses to stimuli like growth factors, hormones or drugs in three dimensional culture models, as opposed to monolayer models, have been reported to better predict the response observed in a living organism and is therefore more appropriate to model the growth and assess the response to external stimuli by normal and tumor cells (Antoni et al., Int. J. Mol Sci. 2015; 16(3):5517-27).

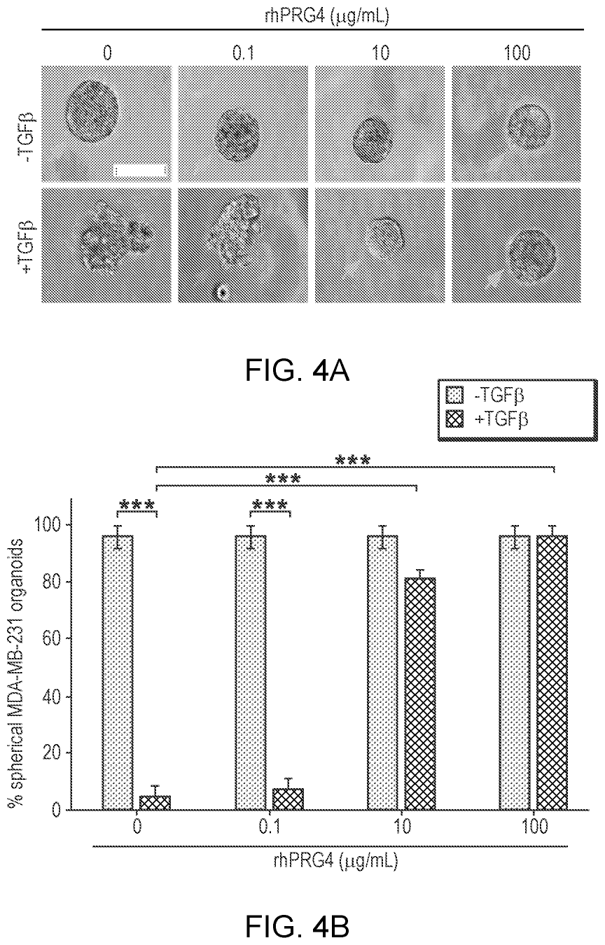

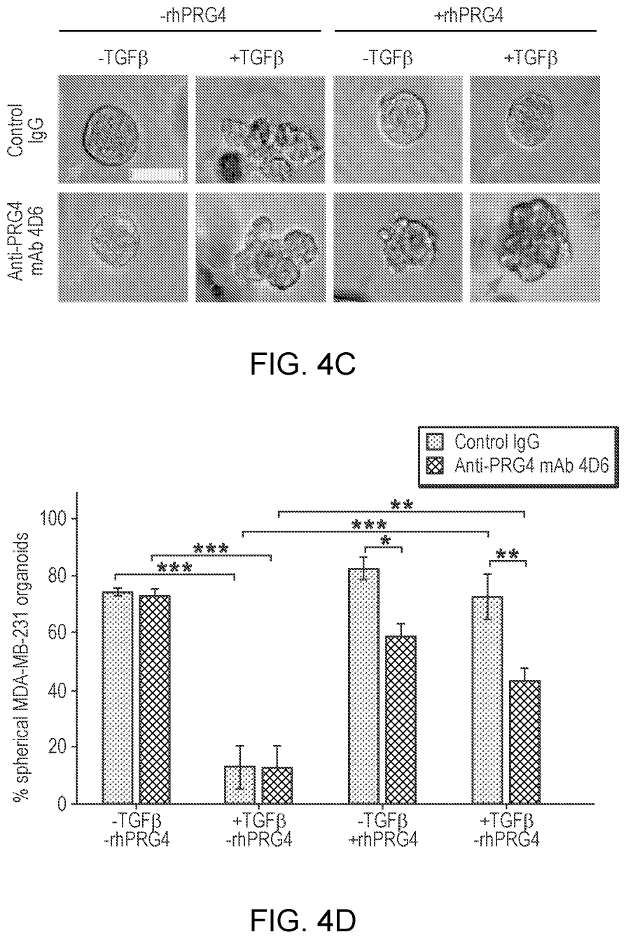

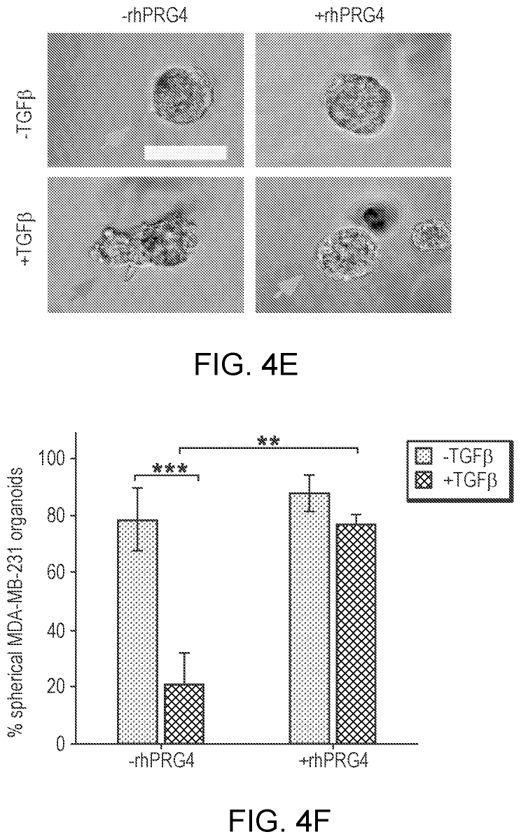

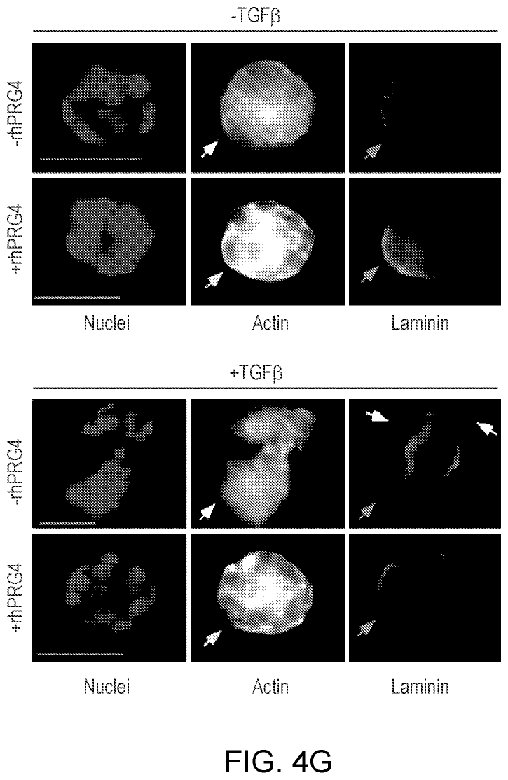

To study the potential anti-invasive effect of rhPRG4 on TNBC-derived cells, we employed a three-dimensional model, where the migratory and invasive behavior of these tumor cells in response to certain stimuli can be easily detected and quantified. The human TNBC MDA-MB-231 breast cancer cell line represents a widely used TNBC cell model for in vitro and in vivo cancer studies including in three-dimensional culture models (Dadakhujaev et al., Oncoscience. 2014; 1(3):229-40; Chanda et al., PloS One. 2017; 12(5):e0177639), thus we used these breast cancer cells in our investigations. As expected, we found that isolated single MDA-MB-231 cells cultured in the context of Matrigel, as an extracellular support system, divided and formed multicellular aggregates that mostly displayed smooth-surfaced spherical phenotypes (FIGS. 4A, 4B). The secreted polypeptide transforming growth factor β (TGFβ) plays a complex role in cancer (Massague, Nat. Rev. Mol Cell. Biol. 2012; 13(10):616-30). In particular, TGFβ can promote migration and invasion, and may thus contribute to cancer metastasis (Massague, Nat. Rev. Mol Cell. Biol. 2012; 13(10):616-30). The ability of TGFβ to remodel extracellular matrix (ECM) components of the tumor microenvironment contributes to its role in increasing migration and invasion of tumor cells (Massague, Nat. Rev. Mol Cell. Biol. 2012; 13(10):616-30). As predicted, we found that TGFβ disrupted the smooth surface and spherical nature of 3D-Matrigel-MDA-MB-231 cell-derived organoids, and promoted an invasive growth of these organoids (Dadakhujaev et al., Oncoscience. 2014; 1(3):229-40; Chanda et al., PloS One. 2017; 12(5):e0177639). Remarkably, rhPRG4 acted in a dose-dependent manner to drastically repress TGFβ-induced invasiveness of 3D-breast cancer cell-derived organoids. The anti-PRG4 monoclonal antibody (mAb) 4D6 specifically recognizes PRG4 (Abubacker et al., Connective Tissue Res. 2016; 57(2):113-23; Chawla et al. Acta biomaterialia. 2010; 6(9):3388-94). Intriguingly, we found that incubation of the 3D cultures with the anti-PRG4 mAb 4D6, but not with mouse IgG control, decreased the ability of rhPRG4 to suppress TGFβ-induced invasive growth of the 3D-breast cancer cell-derived organoids (FIGS. 4C, 4D). Together, these data suggested that rhPRG4 acts in a dose-dependent and specific manner to suppress the invasive behavior of MDA-MB-231 cell-derived organoids.

The findings that PRG4 acts in a specific manner to preserve the non-invasive phenotypes of the TNBC-derived organoids even in the presence of TGFβ, raised the key question whether addition of PRG4 to the Matrigel prior to addition of isolated cells is sufficient to counteract TGFβ-induced invasive growth of breast cancer cell-derived organoids. Remarkably, applying rhPRG4 to the Matrigel even prior to the setting of the three-dimensional culture was sufficient to suppress TGFβ-induced invasive growth of the 3D-TNBC derived organoids (FIGS. 4E, 4F).

Basal lamina disruption and cortical to stress-fiber-like actin reorganization are two requisite factors for cells to become invasive (Akhavan et al., Cancer Res. 2012; 72(10):2578-88, Tojkander et al., J. Cell Sci., 2012; 125(Pt 8):1855-64). It is important to note that the ECM protein laminin is enriched at the basal lamina of organoids maintaining their structural integrity and polarity, and this enrichment is gradually lost with increase in neoplastic tendency (Debnath et al., Nat. Rev. Cancer, 2005; 5(9):675-88, O'Brien et al. Nat. Rev. Mol. Cell. Biol. 2002; 3(7):531-7). Thus, next, the effect of rhPRG4 on the ability of TGFβ to alter the basal lamina status and actin organization in 3D-MDA-MB-231-derived organoids were tested using indirect immunofluorescence analysis (laminin), fluorescently-labelled phalloidin (actin) and Hoechst (nuclei) staining. In untreated MDA-MB-231 cell-derived organoids, the basal lamina was intact as suggested by the appearance of laminin as a solid ring surrounding the outside surface of the organoid, whereas actin was partially cortically oriented (FIG. 4G). TGFβ disrupted basement membrane organization around the organoid as indicated by loss of the laminin ring surrounding the organoids, and promoted actin stress-fiber like appearance. These TGFβ effects are consistent with its ability to increase mobility and invasion of the cellular components of the 3D-MDA-MB-231 organoids (Dadakjujaev et al., Oncoscience. 2014; 1(3):229-40, Chanda et al., Plos One, 2017:12(5):e0177639). Interestingly, rhPRG4 promoted cortical actin organization and solid laminin ring formation in these multicellular structures in the absence or presence of TGFβ. Collectively, these data suggest that rhPRG4 induces an anti-invasive growth behavior of 3D-breast cancer cells-derived organoids grown in the context of an ECM support.

### Example 3: PRG4 Suppresses Invasion and Migration of Breast Cancer Cells

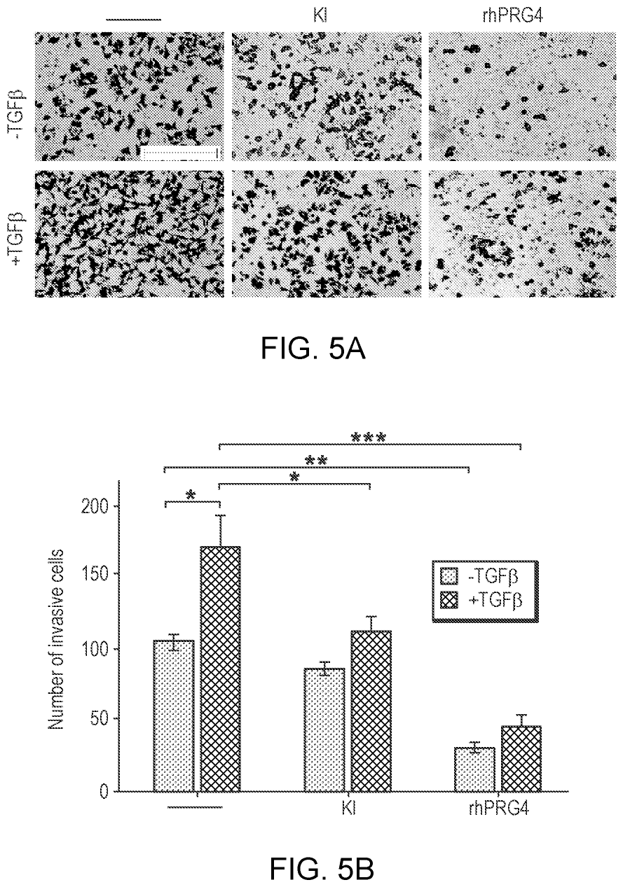

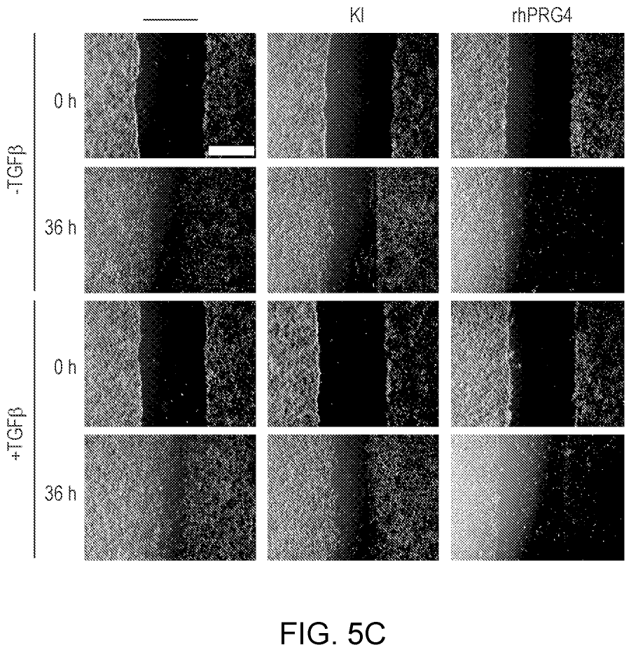

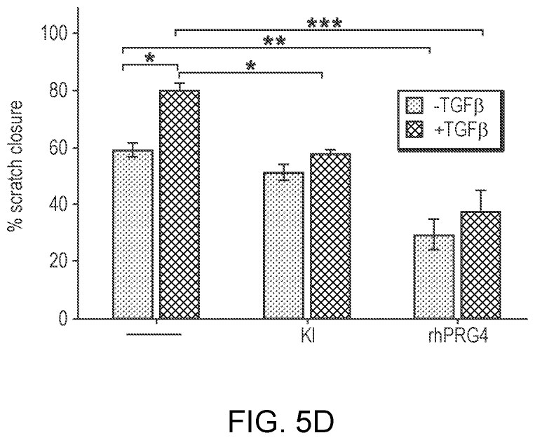

The effect of rhPRG4 on cell invasion was tested using an in vitro transwell assay (Dadakjujaev et al., Oncoscience. 2014; 1(3):229-40). Specifically, serum-starved MDA-MB-231 cells were seeded in the upper chamber on top of a Matrigel-coated membrane, with the lower chamber having 10% FBS-containing growth medium in the absence or presence of TGFβ, alone or together with TGFβ type I ser/thr kinase receptor (TβRI) small molecule kinase inhibitor SB431542 (KI) or rhPRG4 as a chemoattractant (Halder et al., Neoplasia, 2005; 7(5):509-21). TGFβ acted in a TORT-signaling-dependent manner to promote the invasion of MDA-MB-231 cells as compared to untreated control (FIGS. 5A, 5B). Intriguingly, rhPRG4 blocked the ability of MDA-MB-231 cells to be invasive in the absence or presence of TGFβ. In addition to invasion, migration plays an important role in the ability of cancer cells to move to sites outside the primary tumor site for metastasis (Bendas et al., Int. J. Cell Biol. 2012:676731). Thus, in vitro scratch assays were performed to test the effect of PRG4 on migratory behavior of the cancer cells. A scratch was created along the midline of overnight-serum-starved confluent cell monolayers in each well, followed by incubation for 36 h with low-serum medium without or with TGFβ, alone or with KI or rhPRG4. TGFβ signaling increased the scratch closure process as compared to that in the control well, whereas, rhPRG4 significantly delayed the scratch closure process in the absence or presence of TGFβ (FIGS. 5C, 5D). Collectively these results demonstrate that rhPRG4 potently inhibits the invasive and migratory properties of breast cancer cells in vitro.

### Example 4: rhPRG4 does not Regulate TGFβ-Smad Signaling

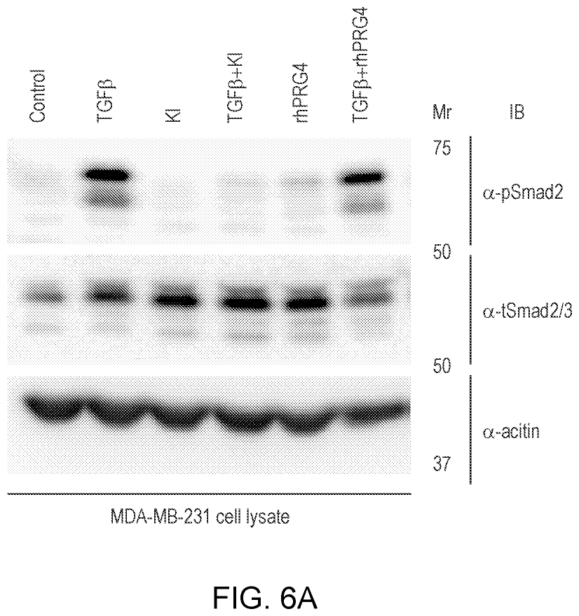

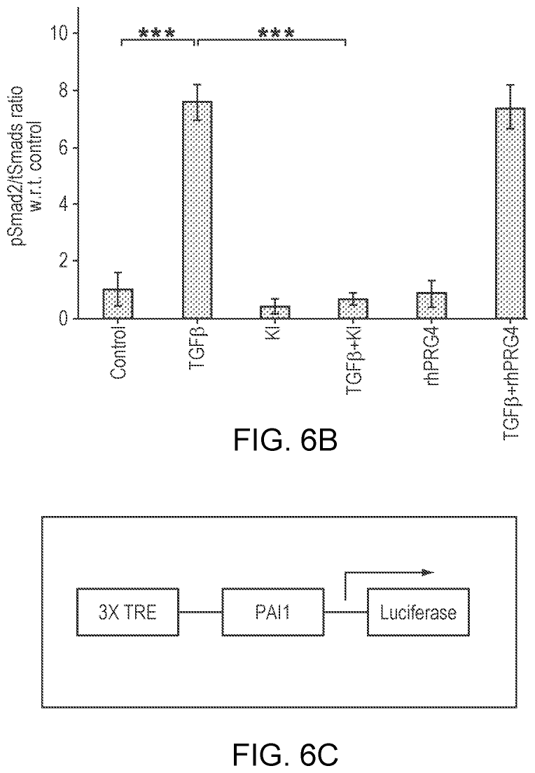

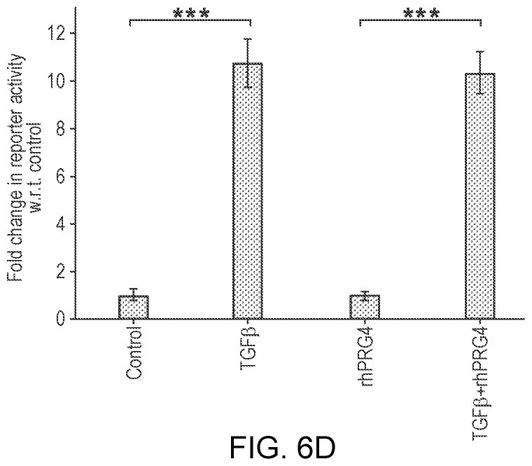

Experiments were performed to determine how rhPRG4 promotes anti-invasive and anti-migratory cellular responses. As rhPRG4 suppressed TGFβ-induced invasion and migration of MDA-MB-231 cells in vitro, the idea that rhPRG4 may antagonize TGFβ signaling pathway was tested. Binding of TGFβ ligands to the cognate receptors on the cell surface, leads to the phosphorylation of receptor-regulated proteins Smad 2 and 3, which are important for downstream TGFβ signaling (Massague, Nat. Rev. Mol. Biol. 2012; 13(10):616-30). Thus, determining if rhPRG4 affects the ability of TGFβ to induce the phosphorylation of Smad2 on its last C-terminal serine residues in these cells was tested. Immunoblotting analyses of lysates of MDA-MB-231 cells, incubated with growth medium without or with TGFβ, alone or together KI or rhPRG4, interestingly revealed that rhPRG4 did not appreciably alter TGFβ-induced phosphorylation of Smad2 on Serine residues 465 and 467 of the C-terminus (FIGS. 6A, 6B).

Further analyses were carried out to ascertain if rhPRG4 affects TGFβ-Smad-dependent signaling. In particular, the effect of rhPRG4 on TGFβ-Smad-induced transcriptional activity using the widely used 3TP-Lux reporter assay in which the expression of the firefly luciferase gene is under the control of three-tandem repeats of a TPA-responsive elements (3T) and promoter elements of the TGFβ-responsive gene the plasminogen activator inhibitor 1 (PAI-1) (Hsu et al., J. Biol. Chem., 2006; 281(44):33008-18) was determined. MDA-MB-231 cells cotransfected with 3TP-lux plasmids together with a CMV-Renilla luciferase plasmid as an internal transfection efficiency control, were incubated overnight with low serum-containing growth medium without or with TGFβ, alone or with rhPRG4, lysed and then subjected to luciferase assays. As expected, TGFβ led to significant induction of 3TP-Lux reporter activity (FIG. 6C, 6D). However, rhPRG4 did not alter TGFβ-induced 3TP-Lux reporter activity in MDA-MB-231 cells. These data are consistent with the lack of effect of rhPRG4 on TGFβ-induced Smad2 phosphorylation. Collectively, these data suggest that rhPRG4 may act downstream of TGFβ-Smad-induced transcription to affect the biological processes of migration and invasion.

### Example 5: rhPRG4 Suppresses Hyaluronan-Induced Invasion of Breast Cancer Cells

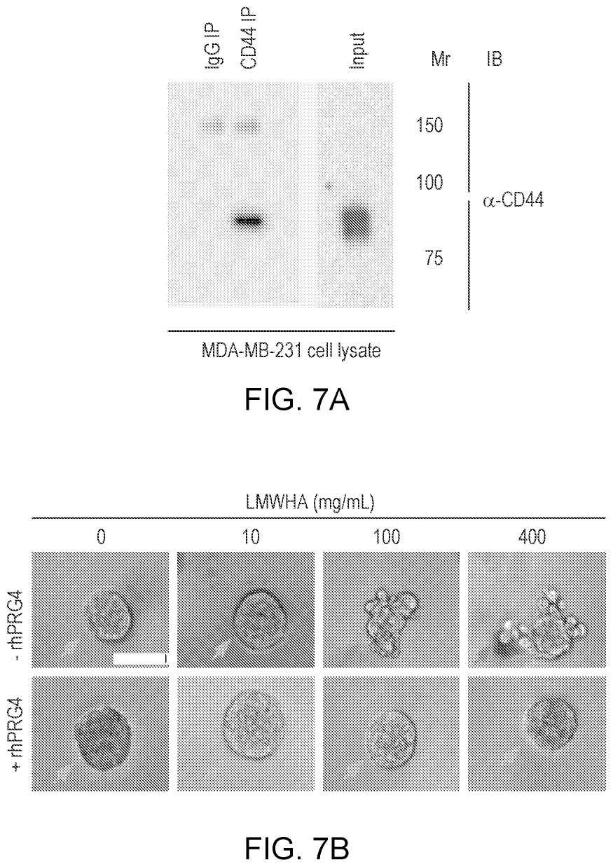

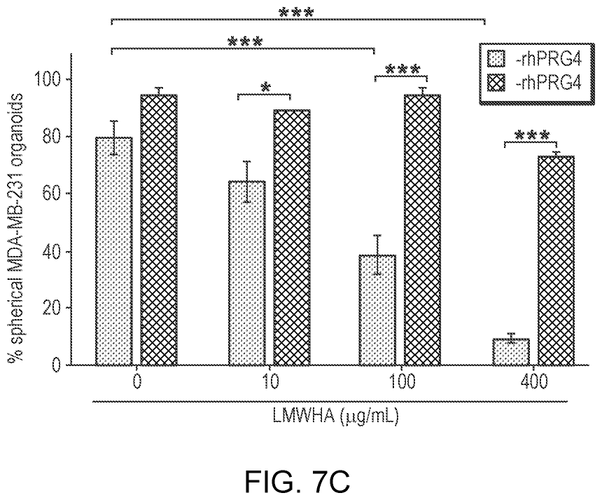

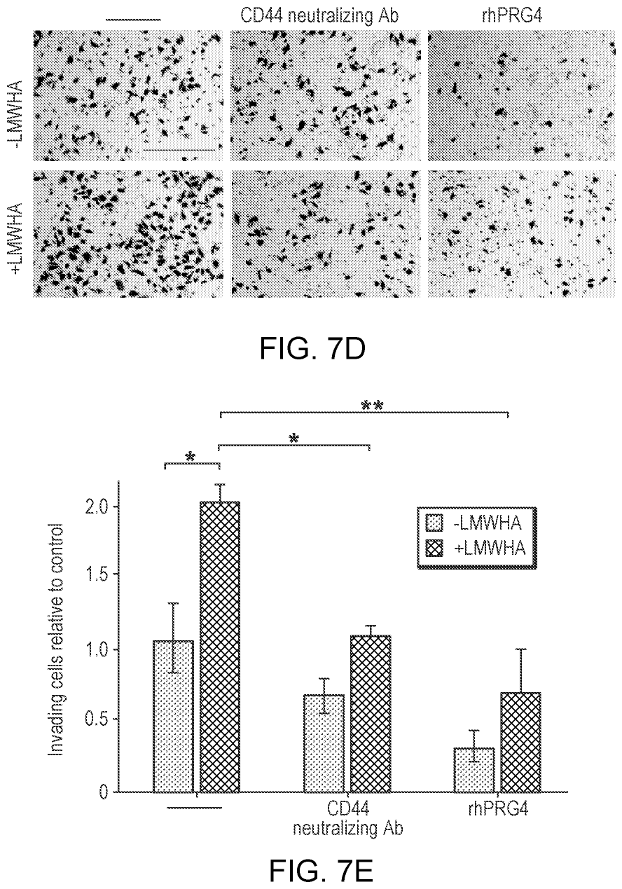

Increasing evidence suggests that a key downstream signaling axis that contributes to TGFβ-induced invasion and metastasis of breast cancer is the hyaluronan (HA)-cluster of differentiation 44 (CD44) pathway (Meran et al., J. Biol. Chem., 2011; 286(20):17618-30; Li et al., Int. J. Mol. Med., 2015; 36(1):113-22; Midgley et al., J. Biol. Chem., 2013; 288(21):14824-38). A number of reports suggest that TGFβ can increase the abundance of hyaluronan synthase 2 (HAS2) enzyme that catalyzes the production and secretion of hyaluronan (HA), especially the low molecular weight hyaluronan (LMWHA) in the stroma (Misra et al., Front. Immunol., 2015; 6:201; Porsch et al., Oncogene, 2013; 32(37):4355-65). In addition, TGFβ is suggested to increase the expression of the HA receptor CD44 on tumor cells (Li et al., Int. J. Mol. Med., 2015; 36(1):113-22). In turn, evidence suggest that LMWHA associate with CD44, triggering the activation of specific signaling pathways that enhance invasion and metastatic ability of tumor cells (Meran et al., J. Biol. Chem., 2011; 286(20):17618-30; Li et al., Int. J. Mol. Med., 2015; 36(1):113-22; Midgley et al., J. Biol. Chem., 2013; 288(21):14824-38). Recent in vitro data support the idea that in addition to being a boundary lubricant and possessing anti-adhesive properties, rhPRG4 may compete with HA for CD44 binding, which may suppress downstream signaling, contributing to the proliferation of osteoarthritis- and rheumatoid arthritis-derived synoviocytes (Alquraini et al., Arthritis Research & Therapy. 2017; 19(1):89; Al-Sharif et al., Arthritis Rheumatol. 2015; 67(6):1503-13). Given these evidence, we asked if rhPRG4 suppression of TGFβ-mediated invasive growth of 3D-breast cancer cell-derived organoid as well as invasiveness and migration of cells detected by trans-well invasion and scratch healing assays (FIGS. 4A, 5A, 5C) involves disruption of a TGFβ-controlled HA-CD44 signaling axis. To address this question, the steady-state protein level of CD44 in the MDA-MB-231 cells was characterized. Immunoprecipitation followed by immunoblotting analysis of cell lysates indicated that CD44 is expressed in the MDA-MB-231 cells (FIG. 7A), raising the possibility of an active CD44-dependent signaling axis. To address this question, the effect of HA on the invasive behavior of the 3D-MDA-MB-231 cells was evaluated using LMWHA (<10 kDa) which has been reported to be elevated in the breast tumor stroma and serum in metastatic disease (Wu et al., FASEB J., 2015; 29(4):1290-8; Peng et al., Intl. J. Cancer, 2016; 138(10):2499-509). The 3D-MDA-MB-231 cells were left untreated or incubated with increasing concentrations of LMWHA either alone or in combination with rhPRG4. LMWHA acted in a dose-dependent manner to increase the proportion of invasive organoids, and as reflected by the decrease in the proportion of spherical organoids (FIGS. 7B, 7C). However, rhPRG4 suppressed the ability of LMWHA to promote the invasive growth of breast cancer cell-derived organoids. Consistent with the results from the 3D-cultures, in transwell invasion assays, we found that LMWHA increased the proportion of invading cells (FIGS. 7D, 7E). On the other hand, a CD44 neutralizing antibody which interferes with the HA-CD44 interaction (Kariya et al., BBA Clinical. 2015; 3:126-34), also blocked cell invasion in the presence of LMWHA. Similarly, rhPRG4 was able to inhibit breast cancer cell invasion in the presence of exogenous LMWHA. Collectively, these results indicate that LMWHA induces invasive behavior in MDA-MB-231 cells, both in 3D culture and transwell invasion assay, which can be blocked by rhPRG4.

### Example 6: CD44 is Crucial for TGFβ-Induced Invasiveness in MDA-MB-231 Cells

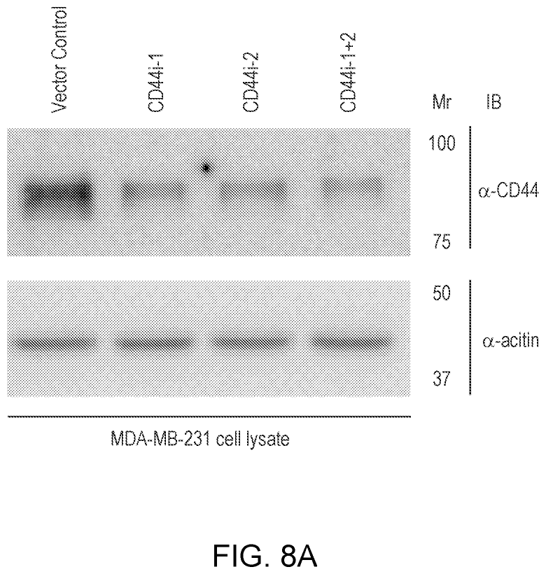

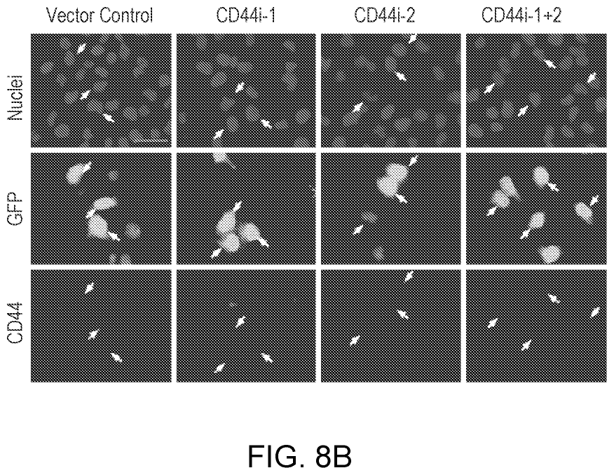

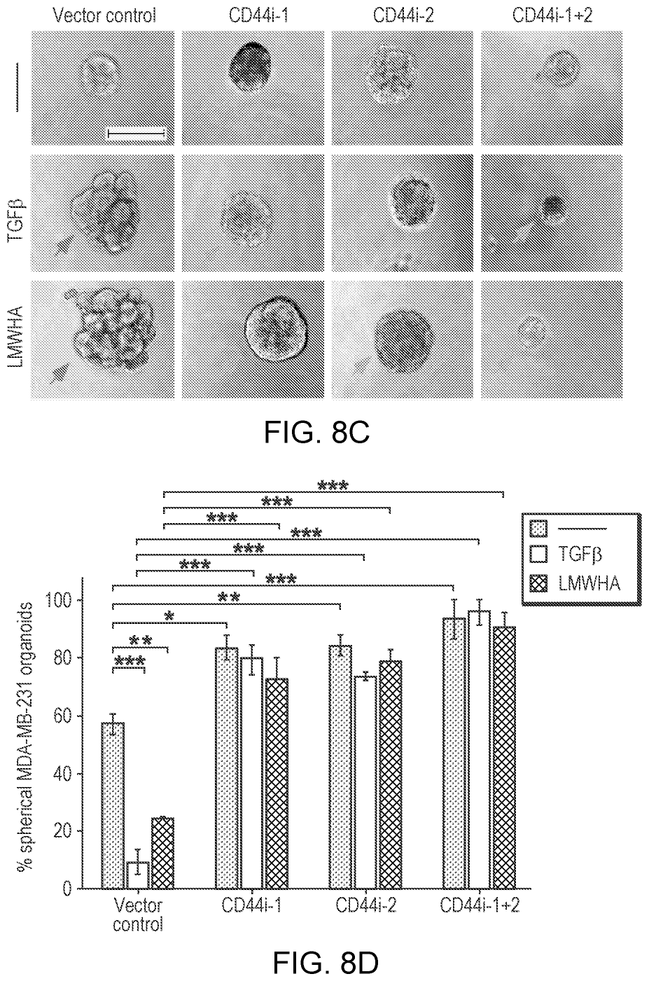

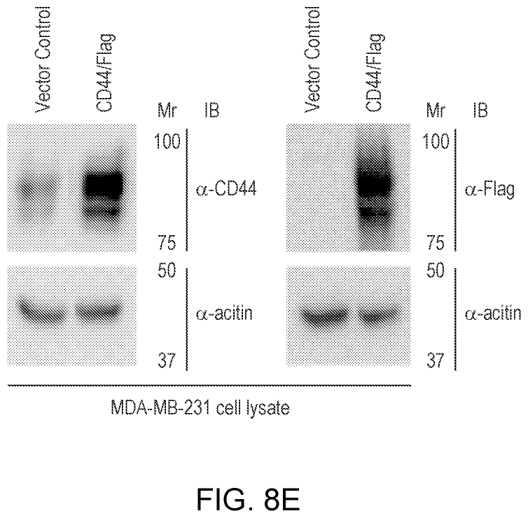

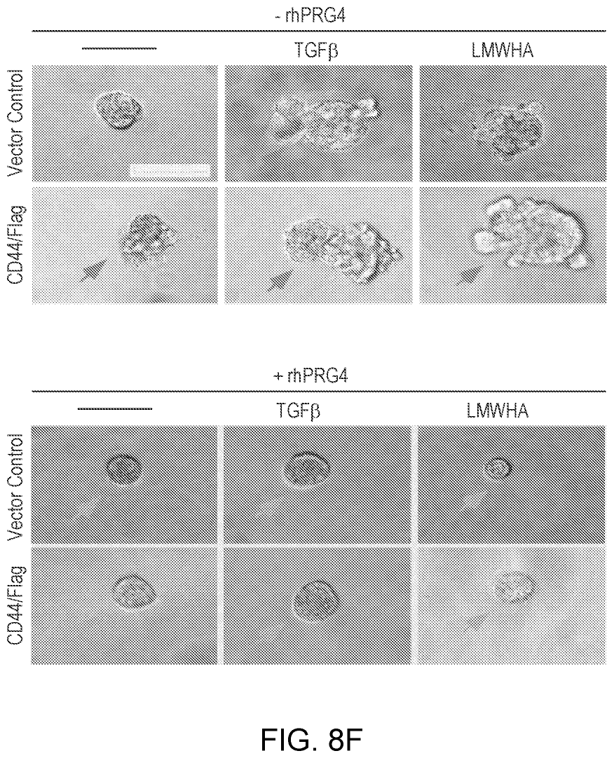

To further investigate whether TGFβ- and LMWHA-mediated invasiveness of the MDA-MB-231 cells are CD44-dependent, an RNA interference (RNAi) approach of gene silencing to knockdown CD44 in these cells was used. Two small hairpin RNAs (shRNAs) were designed targeting specific sequences in exon 16 and exon 4 of CD44, respectively. CD44 immunoblotting of lysates of MDA-MB-231 cells transfected with a control pU6 RNAi vector or RNAi plasmid expressing shRNA1, shRNA2, alone or together confirmed that these shRNAs individually or together efficiently knockdown endogenous CD44 (FIG. 8A). CD44 immunofluorescence analysis of fixed MDA-MB-231 cells transfected with the RNAi control vector or a plasmid expressing CD44i-1/2 showed drastic CD44i-1/2-induced knockdown of endogenous CD44 (FIG. 8B).

Next, the effect of endogenous CD44 knockdown by CD44 shRNA1/2 on 3D-MDA-MB-231 cell-derived organoids in the presence or absence of TGFβ or LMWHA was tested. Interestingly, knockdown of CD44 increased the percentage of spherical organoids, and suppressed the ability of TGFβ or LMWHA to induce an invasive behavior as demonstrated by preservation of spherical organoids. Interestingly, coexpression of CD44i-1 and CD44i-2, consistently, reduced the size of the organoids drastically in the absence or presence of TGFβ or HA. These data suggest that CD44 may mediate TGFβ and LMWHA-induction of invasive growth of MDA-MB-231 cell-derived organoids (FIGS. 8C, 8D).

Having established the importance of the receptor CD44 for TGFβ and LMWHA-induced invasiveness in MDA-MB-231-derived organoids, the effect of overexpression of CD44 on these organoids in the absence or presence of TGFβ and HA was analyzed. First, RT-PCR was used to amplify an open reading frame of CD44 cDNA from the MDA-MB-231 cells and was then subcloned into a CMV-based plasmid to express CD44/FLAG in MDA-MB-231 cells which was confirmed by CD44 and FLAG immunoblotting (FIG. 8E). 3D-organoids were generated from MDA-MB-231 cells transiently transfected with a vector control or with a CD44/FLAG expressing plasmid, and were incubated with growth medium without or with TGFβ, LMWHA, alone or together with rhPRG4. Untreated 3D-organoids derived from vector control transfected cells were mostly spherical and became invasive upon incubation with TGFβ or LMWHA whereas these effects were significantly reversed by rhPRG4 (FIG. 8F, 8G). However, overexpressed CD44/FLAG promoted invasive growth of the 3D-organoids even in the absence of TGFβ or LMWHA. Interestingly, rhPRG4 suppressed the ability of overexpressed CD44 to promote invasive growth of MDA-MB-231 cell-derived organoids in the absence or presence of TGFβ or LMWHA. These results further support the notion that rhPRG4 suppresses breast cancer cell invasive growth in a CD44-dependent manner. Altogether, findings from CD44 knockdown and overexpression studies suggest that rhPRG4 suppresses CD44-mediated TGFβ or LMWHA promotion of an invasive phenotype of the MDA-MB-231 cell-derived organoids.

### Example 7: HA-CD44 Axis Mediates TGFβ-Induced Invasive Growth of Breast Cancer Cell-Derived Organoids

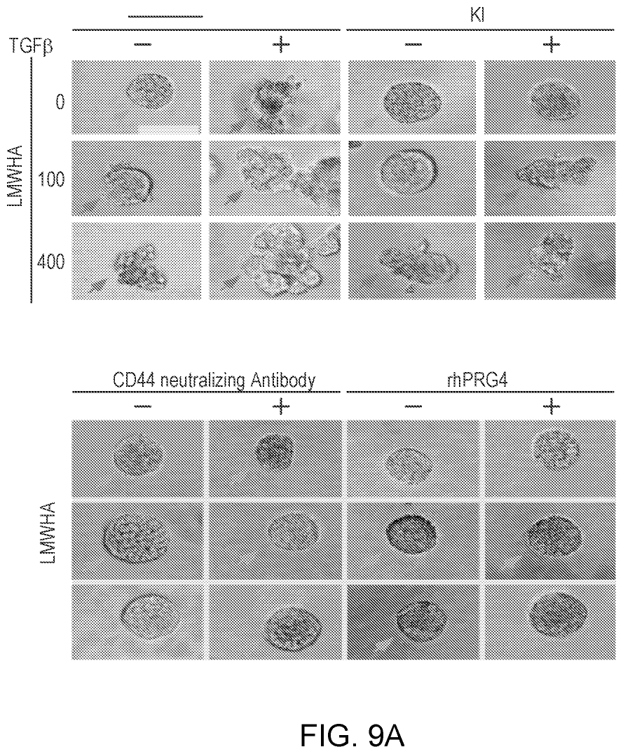

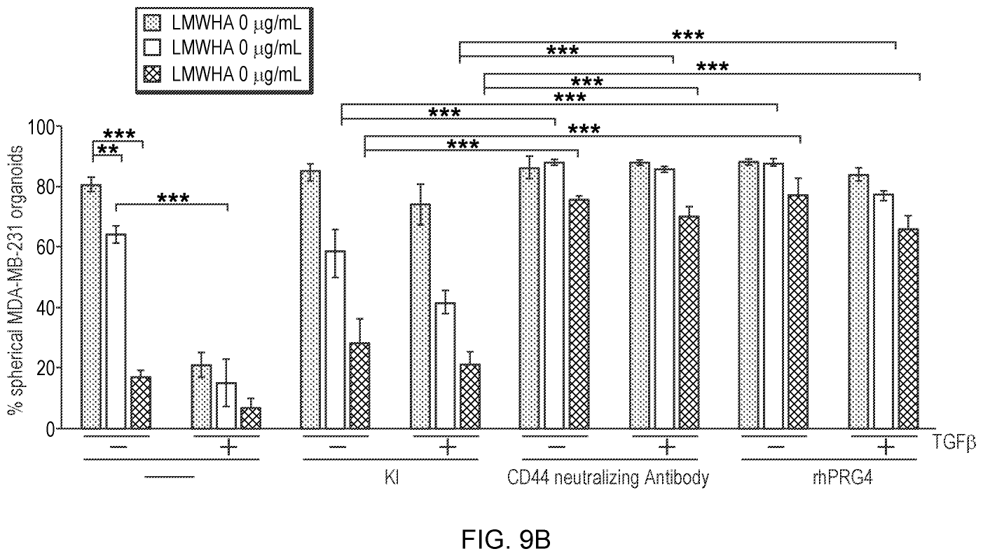

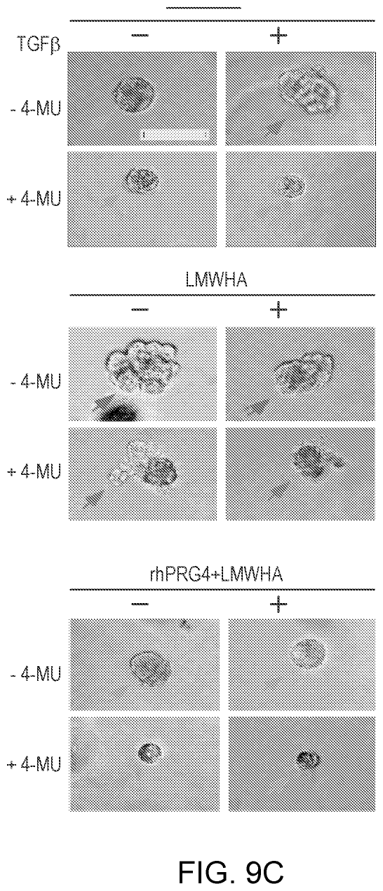

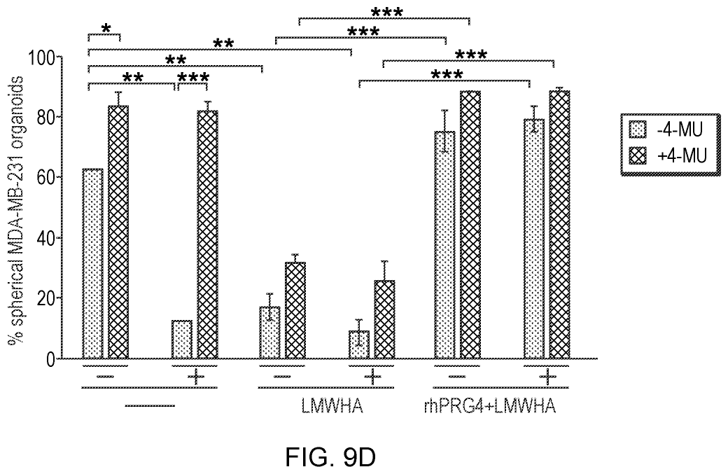

To further investigate the role of CD44 in TGFβ-induced invasive growth of MDA-MB-231 cell-derived organoids, 3D cultures were incubated with growth medium or increasing concentrations of LMWHA, alone or together with different combination of TGFβ, KI, CD44 neutralizing antibody or rhPRG4 (FIGS. 9A, 9B). Consistent with the previous findings, incubation of 3D-MDA-MB-231 cell-derived cultures with increasing concentrations of LMWHA promoted an invasive growth of the breast cancer cell-derived organoids. TGFβ further enhanced the ability of LMWHA to induce invasive growth of these organoids. Interestingly, KI which suppressed the TGFβ-induced invasive growth, could not reverse the LMWHA effect, suggesting LMWHA-mediated invasion acts downstream of the TGFβ signaling pathway. Conversely, the CD44 neutralizing antibody suppressed the ability of both LMWHA and TGFβ stimuli to induce invasive growth, and indeed this intervention significantly increased the number of spherical organoids compared to KI treatment. Furthermore, the blockade of TGFβ-induced invasive growth of these organoids by CD44 neutralizing antibody suggests that TGFβ induces invasiveness in a CD44-dependent pathway in these cells. PRG4, similar to the CD44 antibody, suppressed TGFβ and LMWHA-induced invasive growth of the MDA-MB-231 cell-derived organoids. These data suggest that rhPRG4 is working downstream of both the LMWHA and TGFβ-mediated pathways. Collectively, these results indicate that the HA-CD44 axis contributes significantly to TGFβ-induced invasive growth of MDA-MB-231 cells and this signaling axis appears to be a rhPRG4 target.

To further determine the impact of HA-CD44 pathway on TGFβ-induced invasiveness of these cells, and more specifically the role of HA synthesis by the hyaluronic acid synthase (HAS) enzymes (Auvinen et al., Breast cancer research and treatment, 2014; 143(2):277-86), the effect of 4-methylumbelliferone (4-MU, a HAS inhibitor) on 3D-MDA-MB-231 cell-derived organoids (Urakawa et al., Intl. J. Cancer, 2012; 130(2):454-66) was examined. Specifically, 3D-MDA-MB-231 cell-derived organoids were incubated without or with 4-MU, alone or together with TGFβ, with different combinations of LMWHA and rhPRG4 (FIGS. 9C, 9D). 4-MU suppressed TGFβ-induced invasive growth of 3D-MDA-MB-231 cell-derived organoids, which was reversed by addition of exogenous LMWHA. Conversely, rhPRG4 suppressed LMWHA-induced invasive growth of 4-MU-treated 3D-MDA-MB-231 cell-derived organoids. Collectively, these data indicate that TGFβ induces invasive growth of 3D-MDA-MB-231 cell-derived organoids in an HA-dependent manner. Furthermore, rhPRG4 suppression of both TGFβ and LMWHA-induced invasive growth of 3D-MDA-MB-231 cell-derived spheroids suggest that rhPRG4 acts downstream of HA production and its signaling pathways.

Overall, these results suggest an interplay between TGFβ signaling and HA-CD44 axis in promoting the invasive behavior of 3D-MDA-MB-231 cell-derived organoids. Importantly, this data suggests that rhPRG4 may act downstream of TGFβ to suppress HA-CD44 axis's ability to promote invasive growth of the MDA-MB-231-derived organoids.

### Example 8: HA-CD44 Signaling Axis is Regulated by TGFβ and PRG4

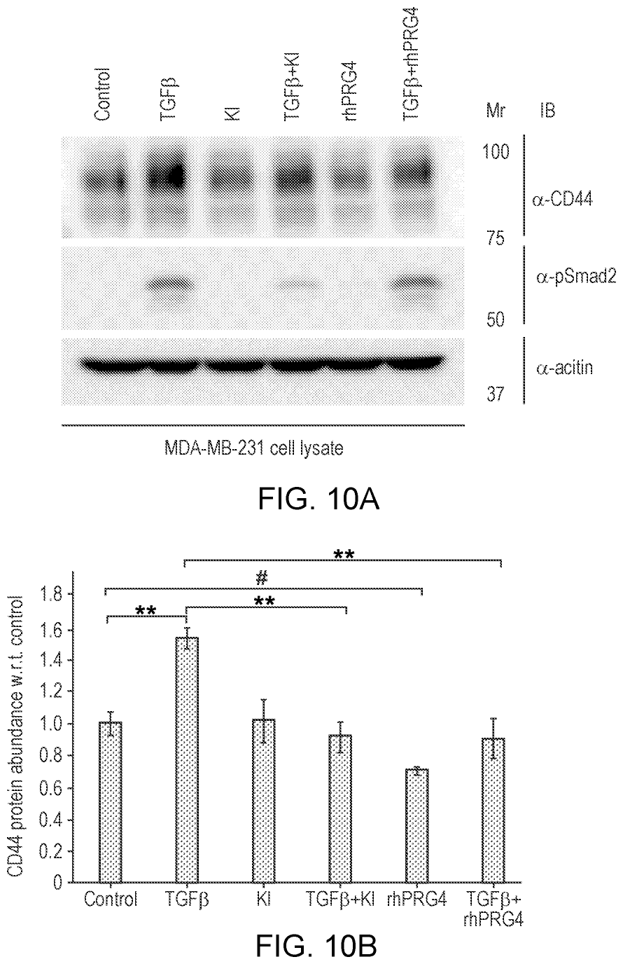

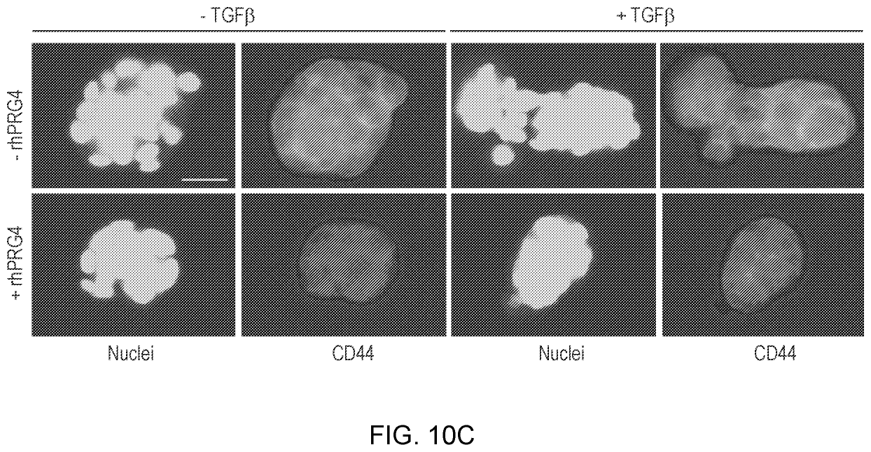

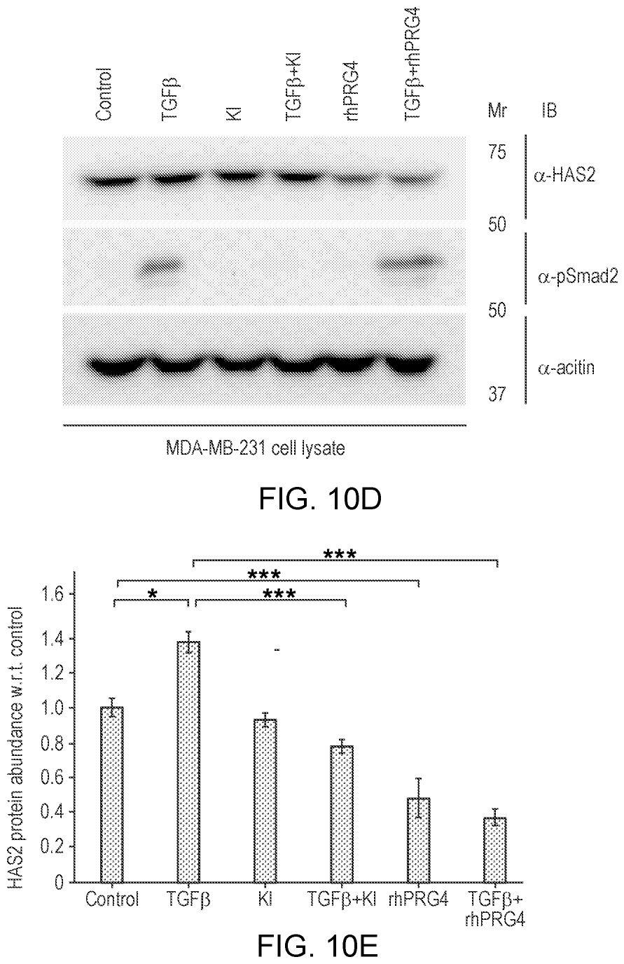

To gain further insight into the molecular mechanisms by which TGFβ may regulate CD44 signaling, the effect of TGFβ on the protein abundance of CD44 in MDA-MB-231 cells was tested using immunoblotting analyses. Interestingly, these experiments showed that activation of the TGFβ-signaling promoted the protein abundance of CD44 in MDA-MB-231 cells (FIGS. 10A, 10B). In contrast, we found that PRG4 reduced the protein abundance of CD44 in the absence or presence of TGFβ. rhPRG4 did not reduce TGFβ-induced Smad2 phosphorylation suggesting that rhPRG4-mediated CD44 suppression may not involve this step of the pathway. In other analyses using CD44 indirect immunofluorescence of untreated, TGFβ and/or PRG4-treated MDA-MB-231 cell-derived organoids revealed that TGFβ enhanced the CD44-immnostaining signal, whereas rhPRG4 reduced this signal in the absence or presence of TGFβ, thus further confirming the immunoblotting data (FIG. 10C).

That the HAS2 inhibitor 4-MU suppressed TGFβ-induced invasiveness of 3D-breast cancer cell-derived organoids suggested that TGFβ may regulate the production of HA in the MDA-MB-231 cells. Thus, we examined the effect of TGFβ on HAS protein levels. Amongst HAS1/2/3 isoforms, HAS2 is the most prevalent enzyme in these MDA-MB-231 cells (Schwertfeger et al., Front Immunol. 2015; 6:236). Immunoblotting analyses showed that TGFβ increased the protein abundance of HAS2 in MDA-MB-231 cells (FIGS. 10D, 10E). Interestingly, rhPRG4 suppressed the protein abundance of HAS2 in the absence or presence of TGFβ.

Importantly, these analyses reveal that in contrast to TGFβ, rhPRG4 leads to reduction in the protein abundance of CD44 and HAS2 in the absence or presence of exogenous TGFβ, which could provide a mechanism by rhPRG4 suppresses TGFβ-induced invasiveness of breast cancer cells.

### Example 9: Summary of Findings from Data Provided in

### Examples 3-8

The data provided in Examples 3-8 demonstrate the novel anti-migratory and anti-invasive roles for the mucin-like glycoprotein rhPRG4 in carcinoma cells derived from a patient with triple-negative breast cancer (TNBC). In particular, by counteracting the ability of the cytokine TGFβ to promote invasive growth of the three-dimensional human MDA-MB-231 TNBC cell-derived organoids, rhPRG4 preserves a non-invasive spherical morphology of these multicellular structures. Epistatic studies revealed that rhPRG4 acts downstream of TGFβ-Smad signaling to achieve its anti-migratory and anti-invasive effects. Moreover, the data suggest that rhPRG4 disrupts TGFβ-induced HA-CD44 signaling activation, which plays a key role in the invasive growth of MDA-MB-231 cell-derived organoids by this cytokine. The data show that, in biochemical analyses, TGFβ signaling increases, while rhPRG4 reduces, in the absence or presence of exogenous TGFβ, the protein abundance of the HA-producing enzyme HAS2 and the HA receptor CD44 in MDA-MB-231 cells, thus pointing to a mechanism by which rhPRG4 inhibits invasion and migration in these cells. Altogether, these findings add important insights into the biological functions and potential therapeutic implications of rhPRG4.

That rhPRG4 exerts anti-migratory and anti-invasive effects in breast cancer cells demonstrates a role for this glycoprotein in epithelial tissue-derived cancers, which represent the majority of solid tumors. These findings contribute important evidence to support the growing idea that rhPRG4 can regulate cellular responses thus acting beyond its original identified role as purely a cartilage boundary lubricant and suggests a new dimension to the emerging biotherapeutic activity of the recombinant glycoprotein.

The finding that anti-PRG4 mAb 4D6, which specifically recognizes PRG4 based on immunoblotting and immunohistochemistry analyses (Abubacker et al., Connective Tissue Res., 2016; 57(2):113-23; Chawla et al., Acta biomaterialia. 2010; 6(9):3388-94), interferes with the ability of rhPRG4 to suppress invasive growth of breast cancer cell-derived organoids suggests that mAb 4D6 acts as an PRG4 neutralizing antibody. This previously unreported anti-PRG4 activity might reflect a hindrance by mAb 4D6 of PRG4 interactions with other molecules with relevance to its anti-invasive activity. Future identification of the epitope in PRG4 targeted by this antibody may add insight into functional domains of rhPRG4 with respect to its anti-invasive properties.

TGFβ plays a dual role in cancer initiation and progression (31, 46). At initial stages of neoplastic disease, evidence suggest that TGFβ acts as a tumor suppressor, while at the later stages of cancer, it can promote invasiveness and metastasis of different carcinomas including breast (Massague, Nat. Rev. Mol. Cell. Biol., 2012; 13(10):616-30; Lebrun et al., ISRN Mol. Biol. 2012:381428). Thus, identifying ways to downregulate the tumor promoting role of TGFβ without affecting its tumor suppressive property may further control tumor growth. The finding that rhPRG4 suppresses TGFβ-induced invasive growth without affecting phosphorylation and the transcriptional activity of the receptor-regulated Smads (R-Smad, e.g. Smad2) raises the possibility that the ability of TGFβ to suppress tumor growth might be intact, which can be the subject of future investigations. That rhPRG4 anti-invasive actions on the MDA-MB-231-derived organoids are mediated by blockade of a LMWHA-CD44 signaling axis may have in vivo relevance. In general, enrichment of the cell surface glycoprotein CD44 in tumor cells including breast is correlated with invasive and metastatic characteristics of the cancer and hence poor prognosis (Zoller, Nat. Rev. Cancer, 2011; 11(4):254-67). HA, which is elevated in different carcinomas including breast cancer stroma (Auvinen et al., Am. J. Pathology, 2000; 156(2):529-36) and blood serum (Wu et al., FASEB J., 2015; 29(4):1290-8; Peng et al., Intl. J. Cancer, 2016; 138(10):2499-509) acts as a ligand for CD44. Depending on the number of the disaccharides repeats, HA is generally classified as high molecular weight hyaluronic acid (HMWHA) and low molecular weight hyaluronic acid (LMWHA) Misra et al., Front. Immunol., 2015; 6:201). Importantly, LMWHA-CD44 binding can trigger activation of distinct signaling pathways that ultimately promote cancer cell invasion, migration and proliferation (Li et al., Int. J. Mol. Med., 2015; 36(1):113-22; Wobus et al., Appl Immunohistochem. Mol Morphol., 2002; 10(1):34-9; Nam et al., Cellular Signaling. 2015; 27(9):1882-94; Liu et al., Cancer Res., 2017; 77(14):3791-801). In addition, LMWHA-CD44 clusters can act to induce remodeling of the stromal ECM at the invasive front of a tumor mass (Yu et al., Genes & Development, 1999:13(1):35-48). The novel finding here that exogenous LMWHA promotes an invasive growth of MDA-MB-231 cell-derived organoids, is consistent with the idea that the elevated LMWHA in the tumor stroma can promote cancer invasiveness (Auvinen et al., Breast cancer research and treatment, 2014; 143(2):277-86). The idea that rhPRG4 negatively affects HA-CD44-induced invasiveness of cancer cells is consistent with other studies suggesting that PRG4 antagonizes HA-CD44-mediated inflammatory signaling that induce synoviocyte proliferation in rheumatoid arthritis and osteoarthritis diseases and a number of inflammatory cytokine production in human and murine macrophages (Alquraini et al., Arthritis Research & Therapy. 2017; 19(1):89; Al-Sharif et al., Arthritis Rheumatol. 2015; 67(6):1503-13; Qadri et al., Arthritis Res & Ther 2018; 20(1):192). Overall, these findings are consistent with the idea that rhPRG4 and LMWHA compete for CD44 binding (Al-Sharif et al., Arthritis Rheumatol. 2015; 67(6):1503-13) and extend them to breast cancer cells and the TGFβ pathway. Future studies could examine rhPRG4's anti-invasive effects in other types of cancer, in particular high CD44-expressing cancer cells in a HA rich stromal environment (Misra et al., Front. Immunol., 2015; 6:201; Zoller, Nat. Rev. Cancer, 2011; 11(4):254-67).

The data herein show that rhPRG4 links TGFβ to HA-CD44 pathway activation in promoting breast cancer invasion. It has been shown that TGFβ promotes the expression of HAS enzymes, particularly HAS2, which results in the accumulation of high levels of HA in the ECM of breast cancer cells (Misra et al., Front. Immunol., 2015; 6:201; Porsch et al., Oncogene, 2013; 32(37):4355-65). Finally, TGFβ has also been shown to promote the expression of CD44 in a Smad-dependent manner (Li et al., Int. J. Mol. Med., 2015; 36(1):113-22; Tripathy et al., Mol. Cell., 2016; 64(3):549-64). In this study, we report that TGFβ increases the protein abundance of both HAS2 and CD44 in MDA-MB-231 cells. Collectively, these results, together with the effect of inhibition of HAS2 and CD44 loss of function analyses, demonstrate that the HA-CD44 pathway plays a key role in mediating the ability of TGFβ to induce invasion and migration of these cells.

rhPRG4's suppression of the ability of overexpressed CD44 to induce invasive growth of the MDA-MB-231 cell-derived organoids may involve reducing HA-CD44 binding, and/or by reduction of CD44 and HAS2 protein levels, as indicated by immunoblotting and immunofluorescence analyses. Our results demonstrated that rhPRG4 can reduce the TGFβ-induced upregulation of CD44 and HAS2 protein abundance in MDA-MB-231 cells without affecting the phosphorylation of Smad2, suggesting it is working downstream of TGFβ-Smad signaling pathway. Whether rhPRG4 suppresses TGFβ-induced HA-CD44 signaling axis by suppressing MAPK activation in these cells, as suggested by previous literature (Li et al., Int. J. Mol. Med., 2015; 36(1):113-22), remains to be elucidated. Moreover, whether rhPRG4 competes with HA for CD44 binding in breast cancer cells, as shown in a previous study with a different cell type (Al-Sharif et al., Arthritis Rheumatol. 2015; 67(6):1503-13), requires further investigation. Whatever the underlying mechanism by which rhPRG4 inhibits TGFβ-HA-CD44-induced invasion of TNBC breast cancer cell might be, it is evident that rhPRG4 can significantly suppress both TGFβ and LMWHA-induced invasiveness of MDA-MB-231 cells.

These data demonstrate that rhPRG4 can suppress TGFβ-induced invasion and migration of MDA-MB-231 TNBC cells in vitro, at least in part through suppression of the HA-CD44 signaling axis' ability to mediate the TGFβ stimuli. Our findings also demonstrate that rhPRG4 can antagonize TGFβ-induced increase in the protein abundance of CD44 and HAS2, which may explain its suppression of TGFβ-induced invasiveness of these cells. Lastly, rhPRG4 also can inhibit LMWHA-induced invasiveness of these cells. Given that TNBC aggressiveness and mortality is correlated with high CD44 expression (Wang et al., Oncology Letters, 2017:14(5):5890-8)), and previous therapeutic strategies which target TGFβ and CD44 signaling axes have shown promise yet with adverse side effects (Misra et al., Front. Immunol., 2015; 6:201; Neuzillet et al., Pharmacology & Ther., 2015; 147:22-31), along with the potential for rapid translational update of rhPRG4 for clinical evaluation, rhPRG4 represents an ideal candidate as a potential biological anti-cancer therapy. In conclusion, these findings contribute to the understanding of PRG4's biological activity in the previously uninvestigated area of breast cancer, and provide the framework for future investigation to target not only breast cancer but potentially other cancers which are dependent on TGFβ and HA-CD44 signaling for their survival, proliferation and metastatic ability (Misra et al., Front. Immunol., 2015; 6:201; Zoller, Nat. Rev. Cancer, 11(4):254-67).

### Example 10: Treatment of a Human with Triple Negative Breast Cancer

A human female diagnosed with non-metastatic triple negative breast cancer is administered recombinant human PRG4 (rhPRG4) by local injection to the area of the tumor. Administration is done under local anesthesia. A total amount of 2 mL of rhPRG4 in physiological saline at a concentration of 100 ug/mL is administered via a 14 gauge needle. Several injections are made into the tumor and over its surface in order to disperse the dose throughout the tumor area. The dose is administered once weekly for four weeks. After four weeks, a CT scan reveals that the tumor has shrunk in size.

### Example 11: Treatment of a Human with Prostate Cancer

An 80 kg human male diagnosed with colon cancer is administered recombinant human PRG4 (rhPRG4) by systemic administration via a central venous catheter. A total amount of 500 mg of rhPRG4 in 250 mL of physiological saline at a concentration of 2.0 mg/mL of lubricin is administered to create a blood concentration of approximately 100 μg/mL. The dose is administered once weekly for four weeks. After four weeks, a CT scan reveals that the tumor in the colon has decreased in size.

### Example 12: Treatment of a Human with Triple Negative Breast Cancer

A human female diagnosed with non-metastatic triple negative breast cancer is administered recombinant human PRG4 (rhPRG4) by local injection to the area of the tumor. Administration is done under local anesthesia. A total amount of 2 mL of rhPRG4 in physiological saline at a concentration of 100 ug/mL is administered via a 14 gauge needle. Several injections are made into the tumor and over its surface in order to disperse the dose throughout the tumor area. The dose is administered once weekly for four weeks. During the dosing rhPRG4 schedule, the patient also receives a cocktail of taxane and doxorubicin per standard protocols. After four weeks, a CT scan reveals that the tumor has shrunk in size and at a rate faster than in patients being treated solely with the taxane and doxorubicin.

### Example 13: Treatment of Hepatocellular Carcinoma with Lubricin

Hepatocellular carcinoma (HCC) is one of most frequent and lethal neoplasms, and often proves refractory to currently employed therapies. Although Sorafenib and Regorafenib administration is among the preferential drug-based approaches to treat this cancer, overall it does not result in a satisfactory benefit. However, coupling these drugs with various other compounds to enhance their effectiveness is a promising strategy that is being increasingly pursued. On the basis of microarray analysis of tumoral and peritumoral tissues from 78 HCC patients, this example shows that lubricin (PRG4) is expressed in HCC and more importantly that it is strongly correlated (p<0.001) with increased survival of HCC patients. Human cancer-associated fibroblasts (CAFs) isolated from patients with HCC produce Lubricin protein, and human HCC specimens treated with transforming growth factor beta (TGFβ) in culture medium released Lubricin. More importantly, this example shows that treatment with full length recombinant human PRG4 (rhPRG4) dramatically enhances the ability of Sorafenib and Regorafenib to inhibit HCC cell proliferation, although it does not significantly affect cell growth. These data suggest a potential novel tumor-limiting role for TGFβ, and also that lubricin may exert a tumor-suppressive function. Accordingly, this example shows that non-synthetic physiologically-produced compound rhPRG4, alone or in combination with Sorafenib and Regorafenib, may act as an anti-tumor agent and be of value in the treatment of HCC.

Hepatocellular carcinoma (HCC) is among the most frequent causes of cancer-related death worldwide. As the majority of patients with HCC are not eligible for curative therapies, based on surgical approaches or radiofrequency ablation, systemic drug-based therapy is necessary. However, multi-year experience in the administration of compounds such as sorafenib and regorafenib has yielded disappointing results in terms of overall survival. (Kudo et al., Lancet 2018. doi:10.1016/S0140-6736(18)30207-1; Bruix et al. Lancet 2017. doi:10.1016/50140-6736(16)32453-9; Abou-Alfa et al. N Engl J Med 2018. doi: 10.1056/NEJMoa1717002; Kok et al. Cancers (Basel) 2019. doi:10.3390/cancers11070985). A poor knowledge of the molecular mechanisms underlying tumor progression of HCC, as well as the high inter- and intra-tumor heterogeneity exhibited by this disease because of the concomitant preexisting liver disease, hampers the development of a precision medicine approach. (Nault et al., Clin Cancer Res 2015. doi:10.1158/1078-0432. CCR-14-2602; Torrecilla et al., J Hepatol 2017. doi:10.1016/j.jhep.2017.08.013; Hung et al., 2019 doi:10.1007/978-3-030-21540-8_14). The cross-talk between the epithelial cancer cells and the surrounding microenvironment, in particular the Cancer Associated Fibroblasts (CAFs), is believed to play a key role in HCC progression and consequently exerts a critical influence on the clinical outcome. (Kudo 2018 supra, Bruix 2017 supra).

The origin of CAFs is still a matter of some debate. Diverse intrahepatic cell types are suggested to undergo phenotypical changes, ultimately differentiating into CAFs during the inflammatory and progressive fibrogenic evolution of chronic liver disease, as well as during HCC development. In this context, CAFs may be phenotypically programmed by adjacent malignant cells and, in turn, enhance HCC cells proliferation and spread, likely through the deposition or secretion of different molecules including extra-cellular matrix (ECM) proteins (Mazzocca A et al., Hepatology 2011. doi:10.1002/hep.24485; Mazzocca et al., Hepatology 2010. doi:10.1002/hep.23285). Indeed, CAFs are the major contributors of ECM deposition, including fibrillar collagens like type I and III non-collagenous glycoproteins like fibronectin, laminin, hyaluronan, elastin, and proteoglycans. (Bataller R et al., J Clin Invest 2005. doi:10.1172/JCI200524282). The.Proteoglycans (PGs) are a class of heavily glycosylated high molecular weight proteins, that are widely expressed in all fibrotic tissue, in particular in the cartilage tissues, where they have a lubrication function, allowing sliding of the joints. Some PRGs, such as versican (VCAN), are up-regulated by transforming growth factor (TGF)-β, and, surprisingly, contribute to cancer invasion. (Nikitovic et al., IUBMB Life 2006. doi:10.1080/15216540500531713; Cross et al. Prostate 2005. doi:10.1002/pros.20182). Recently, it was shown that the human recombinant form of the proteoglycan lubricin inhibits TGFβ-mediated invasiveness of breast cancer cells by acting on the CD44 axis. “Sarkar et al. PLoS One 2019. doi:10.1371/journal.pone.0219697).

TGFβ has been widely reported to promote a more invasive and aggressive phenotype in HCC. (Mazzocca 2010 supra; Fransvea et al., Hepatology 2008. doi:10.1002/hep.22201; Fransvea et al., Hepatology 2009. doi:10.1002/hep.22731; Mazzocca et al., Hepatology 2009. doi:10.1002/hep.23118; Fransvea et al., Cancer Chemother Pharmacol 2011. doi:10.1007/s00280-010-1459-x). A TGFβ pathway inhibitor, Galunisertib, was reported to be effective in a multicentric clinical trial in patients with advanced HCC. (Abou-Alfa et al. N Engl J Med 2018. doi:10.1056/NEJMoa1717002). Furthermore, in preclinical experimental models, inhibiting TGFβ signaling decreased HCC aggressiveness, reducing CD44 expression. (Rani et al., Cell Death Dis 2018. doi:10.1038/s41419-018-0384-5). In light of the potential anti-tumor function of lubricin, this example investigates its role in a patient setting, as well as its function to limit the growth of HCC cells and to enhance the in vitro cell growth-inhibitory potential of sorafenib and regorafenib.

### Materials and Methods

Cells and reagents: HLE and HLF cell lines were purchased from JCRB Cell Bank (Japan). Hep3B and PLC/PRF/5 cell lines were purchased from ATCC (USA). All these cell lines were cultured in DMEM (Dulbecco's Modified Eagle Medium) supplemented with Sodium Pyruvate, antibiotic-antimycotic, Hepes and 10% fetal bovine serum (FBS) (Thermo Fisher Scientific). Full length recombinant human PRG4 (rhPRG4) was provided from Lubris Biopharma (Weston, Mass., USA).

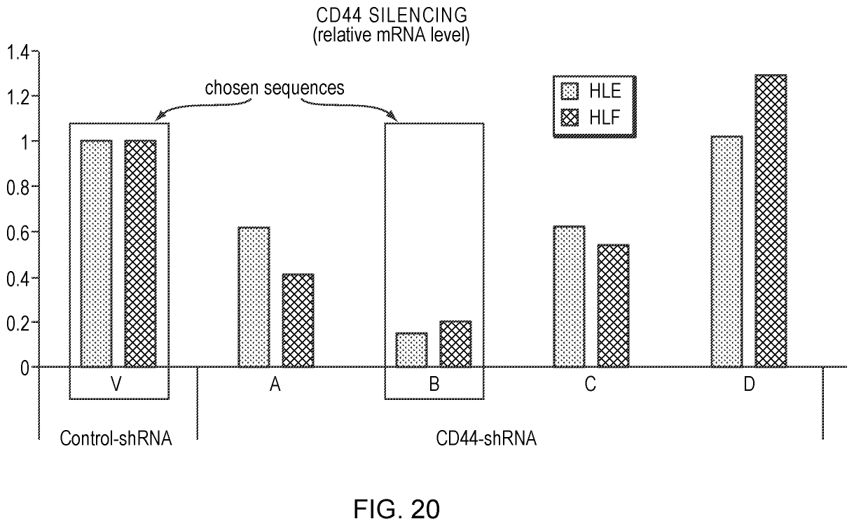

Stable CD44 silencing: HLE and HLF cell lines were transduced with lentiviral particles carrying control non-targeting (V), or specific CD44-targeting shRNA sequences (A to D), and selected with puromycin dihydrochloride (Thermo Fisher Scientific) to obtain stable CD44 silencing, according to manufacturer instructions (OriGene Technologies, Inc., Rockville, Md. 20850, USA). Control-shRNA sequence (V) and CD44-shRNA sequence B were used in all the experiments involving CD44 downregulation. CD44 silencing efficiency is shown in FIG. 20.

Proliferation assay: In 100 μl complete medium, 3,000 cells were seeded in wells of a 96-well plate and left overnight to allow complete attachment. The next day (at time t=0 hours), the medium was removed and replaced with 100 μl of fresh medium in the presence/absence of Sorafenib (2.5 μM), Regorafenib (2.5 μM), rhPRG4 (12.5 to 100 μg/ml). Dimethyl sulfoxide (DMSO) and PBS+0.01% Tween-20 were used as vehicles of Sorafenib/Regorafenib, and rhPRG4, respectively. Cells in triplicate wells for each cell line were fixed at t=0 hours with 4% paraformaldehyde (PFA, pH 7.6, 10 min incubation), then stained with crystal violet (CV) and thoroughly washed to remove the excess staining. After 72 hours, the cells were fixed by adding 100 μl of 4% PFA directly to the medium (2% final PFA concentration, 20 min incubation), and processed for CV staining as previously described. Then, 100 or 200 μl of 1% sodium dodecyl-sulphate were added to the wells and the plates were left under shaking until complete CV release from stained cells. Optical density was at 595 nm wavelength measured using a plate reader.

Immunofluorescence: A cryostat microtome was used to cut 5 μm thick slices of HCC tumor samples. Slices were incubated with blocking buffer (10% FBS in RPMI) for 30 min to minimize nonspecific antibody binding, then incubated for 2 hours with primary antibodies diluted in the same buffer, washed 3 times with PBS (each wash for 5 min under shaking) and finally incubated with secondary AF488- or AF594-conjugated antibodies. At the end of this step the slices were washed four times as previously described, and mounted with DAPI-supplemented Vectashield anti-fade mounting medium.

Adhesion assay: Diluted in 100 μl of serum free DMEM medium (+0.5% BSA), 50,000 cells, were seeded onto uncoated, rhPRG4, or fibronectin (FN)-coated wells of a 96-well plate, and then incubated at 37° C., 5% CO₂ for 30 minutes. An equal volume of 4% PFA (pH 7.2 in PBS) was added and the plates were immediately flicked for a few seconds to allow mixing. Thirty minutes later, the medium was removed and the adherent cells were stained with crystal violet for 10 min. After abundant washing with tap water and distilled water the stained cells were allowed to dry out, and then they were solubilized with 100 μl of 1% sodium dodecyl sulphate in water. Absorbance was read at 595 nm and proportionally related to the number of adhered cells.

Trans-well migration assay: The assay was performed as previously described. (Dituri et al., PLoS One 2013. doi:10.1371/journal.pone.0067109). Briefly, 15,000 cells were loaded onto the top chamber of the trans-well, whose membrane was previously coated with fibronectin on the lower surface, and left to migrate for 16 hours in the presence/absence of rhPRG4 (25 μg/ml) diluted in serum-free DMEM medium (+0.5% BSA) in the lower chamber. The cells were then paraformaldehyde-fixed and stained with crystal violet. Five fields/membrane were captured and the number of cells/field was measured.

Western blot: Tissue proteins were extracted using T-PER Tissue Protein Extraction Reagent Supplemented with Halt Protease and Phosphatase Inhibitor Cocktail EDTA-free (Thermo Fisher Scientific). Briefly, proteins were extracted using a tissue homogenizer. The lysates were incubated on ice for 30 min and vortexed every 10 min. Then, the samples were clarified through centrifugation at 13,000 rpm (at 4° C.) for 20 min to precipitate insoluble debris. The supernatants (containing the extracted proteins) were assayed for protein concentration using Bradford Reagent (Bio-Rad). The proteins were then mixed with Laemmli buffer and 10% β-mercapto ethanol (BME), and denatured at 95° C. for 5 min. Ten to 20 μg of total proteins were loaded onto 4-20% PAA gels and run in SDS-PAGE.

RNA extraction and cDNA synthesis: Thirty to 60 μg of frozen ex-vivo treated HCC tissues were ground with a mortar-pestle in the presence of liquid nitrogen until a thin powder was obtained. The ground tissues were lysed with 0.5-1 ml of RLT buffer+1% (BME) and then processed according to the manufacturer's recommendations (RNeasy kit, Qiagen). RNA isolation from CAFs was performed following the procedure suggested by the RNeasy kit handbook. The obtained RNA was assayed for quality and concentration using the NanoDrop 2000/2000c (Thermo Fisher Scientific). cDNA was synthetized using the High Capacity cDNA reverse transcription kit (Thermo Fisher Scientific), according to relative datasheet.

Real time PCR: One ng/μl of cDNA was used in total 20 μl reaction mix in the presence of 500 nM of each forward and reverse primer referred to a specific gene of interest, and 2×SYBR green master mix (Bio-Rad). The reaction was conducted in a CFX96 Touch Real-Time Detection System (Bio-Rad).

CAFs isolation: Immediately after surgical resection, HCC tumoral and peritumoral specimens were minced into 0.5-1 cm pieces and left in MACS Tissue Storage Solution (Miltenyi Biotec). The tissues were then further cut into smaller size pieces (1-2 mm), washed three times in Hanks balanced salt solution (HBSS), and then incubated in HBSS in the presence of type IV collagenase (Thermo Fisher Scientific) and 3 mM CaCl₂ at 37° C. under gentle rotation for 4 hours. At the end of this step, the dissociation was mechanically facilitated by pipetting up-down the digested tissues with a large size orifice 50 ml pipette. The floating cells were collected and washed three times with HBSS and seeded in normal culture conditions in IMDM+20% FBS. The decanted, partially digested tissue specimens were subjected to a second round of digestion (as previously described). The resulting dissociated cells were washed with HBSS and cultured in IMDM+20% FBS. These cells underwent 2-3 passages to eliminate epithelial/immune/non-adherent cells. To assess the purity of CAFs preparations, immunofluorescence or flow cytometry analyses were performed to evaluate the expression of mesenchymal markers (vimentin and αSMA). The presence of minimal contaminating non-fibroblastic cells (mostly cancerous hepatocytes, cholangiocytes and macrophages) was evaluated using antibodies to EpCAM, CD45, and CD11b.

CAFs treatments and lubricin immunodepletion of CAFs conditioned medium: CAFs were treated for 48 hours in the presence/absence of TGFβ1 (Peprotech) at the final concentration of 5 ng/ml in complete IMDM medium (+20% FBS), then washed three times with serum-free medium, and incubated in serum-free medium for another 48 hours for secretome enrichment. The conditioned medium was then collected, concentered using a centricon device (3 kDa cut-off, Merck-Millipore), and incubated with anti-lubricin, or isotype antibody-bound PBS pre-washed magnetic microbeads, according to the manufacturer's instructions (SureBeads Protein B, BioRad). The lubricin depleted or non-depleted medium was then assayed for protein concentration, and used for further tests.

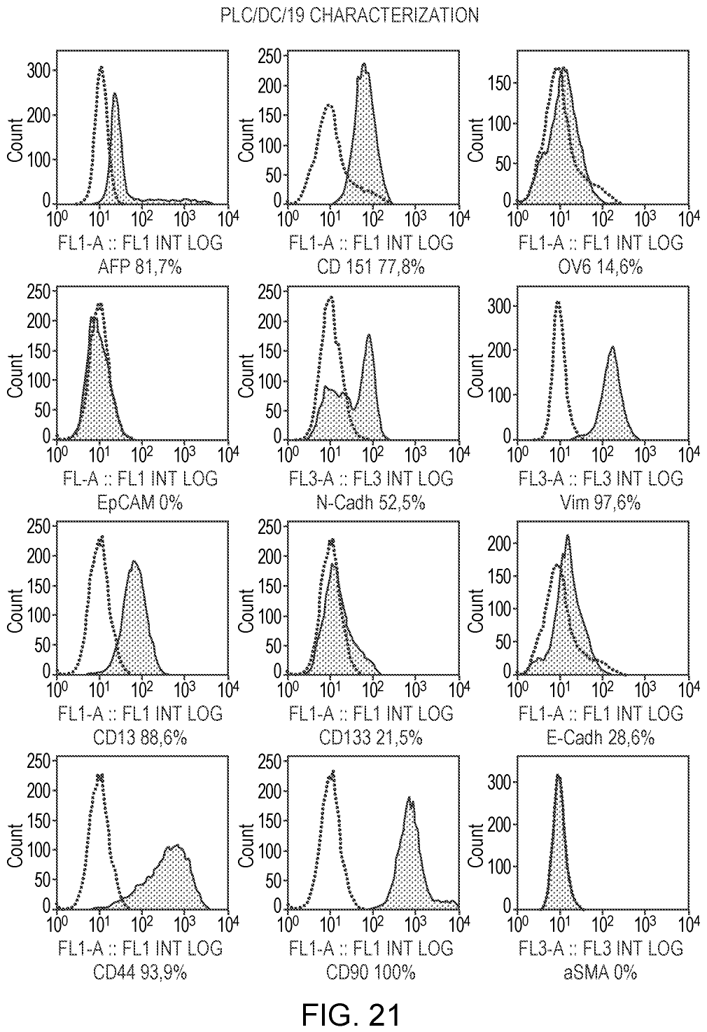

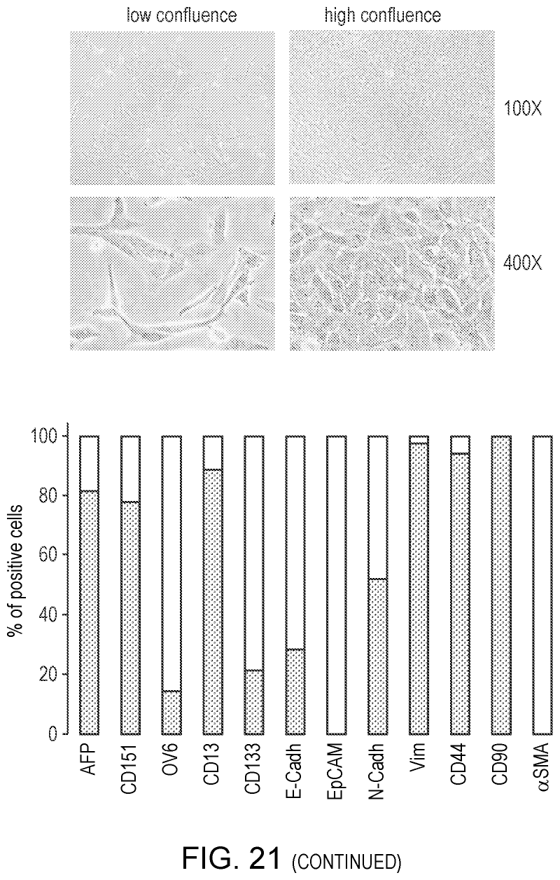

Characterization of the primary HCC cells line PLC/DC/19: The primary HCC cell line, PLC/DC/19, was isolated from freshly collected surgically resected HCC specimen following the same isolation procedure to isolate CAFs. The immunophenotypic characterization of cells was carried out after several (<10) culture passages, by using antibodies to detect stemness markers (OV6, CD133, CD44 and CD90), epithelial markers (AFP, E-Cadh, EpCAM), mesenchymal markers (Vim, N-Cadh, αSMA), and other cancer-related surface proteins (CD13, CD151) (FIG. 21).

Microarray analysis: Microarray analysis was performed as already described (gut 2016). Briefly, using Trizol (Invitrogen, Carlsbad, Calif., USA), total RNA was isolated from non-tumoral (NT) and tumoral (T) liver tissues obtained in a cohort of prospectively enrolled patients at first identification of HCC. RNA was processed using 4×44K whole genome oligonucleotide-based gene expression microarrays (Agilent Technologies, Palo Alto, Calif.; Genomics Service Department of Miltenyi Biotec GmbH Bergisch Gladbach, Germany). Labeling and hybridization procedures were performed according to the instructions provided by Agilent, using the Quick Amp Labeling Kit and the One Color Microarray-Based Gene Expression Analysis Protocol. After RNA conversion into cDNA, during a labeling and amplification step cDNA was converted into cRNA and labeled with Cy3-CTP. Once purified, cRNA were hybridized to Agilent Whole Human Genome Oligo Microarrays 4×44K. After quantification of the signal and normalization of the results using a linear lowness method, data were imported into Resolver software (Rosetta Biosoftware, Kirkland, Wash.) for database management, quality control, and analysis. The gene expression data are available at the Gene Expression Omnibus website (www.ncbi.nlm.nih.gov/geo) under accession number: GSE54236.

Statistical analysis: _Survival analysis. The Kaplan-Meier method was used to estimate the cumulative probability of overall survival. Patients were censored at the time of LT, death, or last available follow-up. Differences in observed probability were assessed using the log-rank test.

### Results

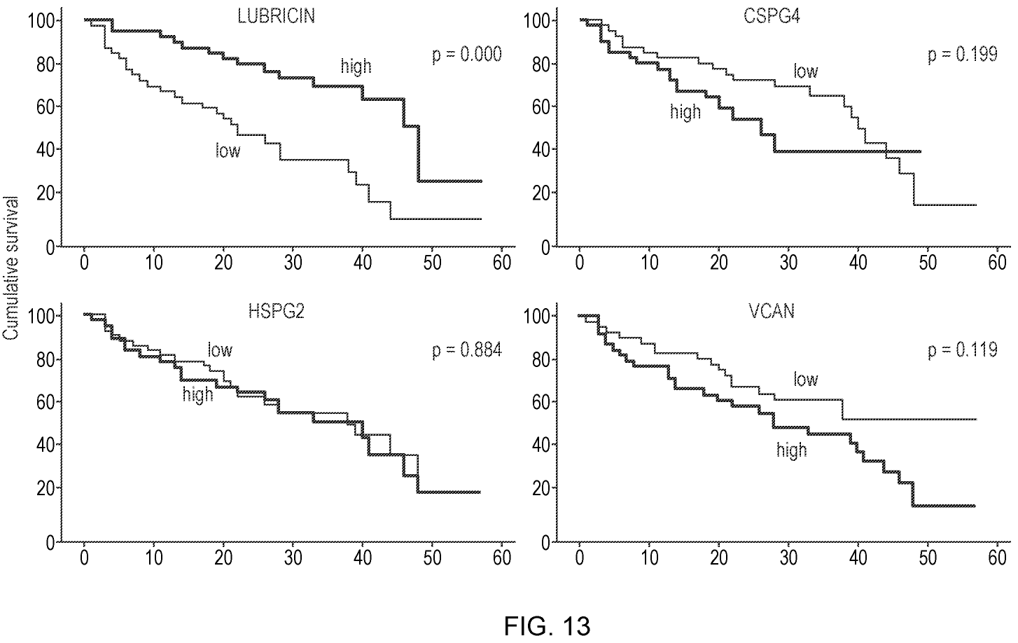

PRGs expression levels in tumoral tissues of HCC patients: We analyzed the expression levels of different PRGs including Chondroitin Sulfate Proteoglycan 4 (CSPG4), Perlecan (HSPG2), Versican (VCAN) and lubricin (PRG4) in a cohort of 78 prospectively recruited HCC patients, coupled with matched microarray gene expression analysis of paired tumoral and peritumoral tissues from the same subjects. Patients were stratified according to mRNA expression levels of each PRG above or below the median values. Expression values of genes of interest in tumors were subtracted from the values in peritumoral tissues to obtain the net gene expression changes, normalized to the background peritumoral expression of the same genes, as previously reported. (Villa et al., Gut 2016. doi:10.1136/gutjnl-2014-308483). Remarkably, patients with higher expression levels of lubricin survived longer, as shown by Kaplan-Meier analysis, compared to those with lower expression levels (p=0.000), whereas CSPG4, HSPG2, and VCAN, did not show any significant correlation with survival (FIG. 13). Accordingly, this example shows that lubricin is expressed in HCC tissues, and high levels are correlated with longer survival.

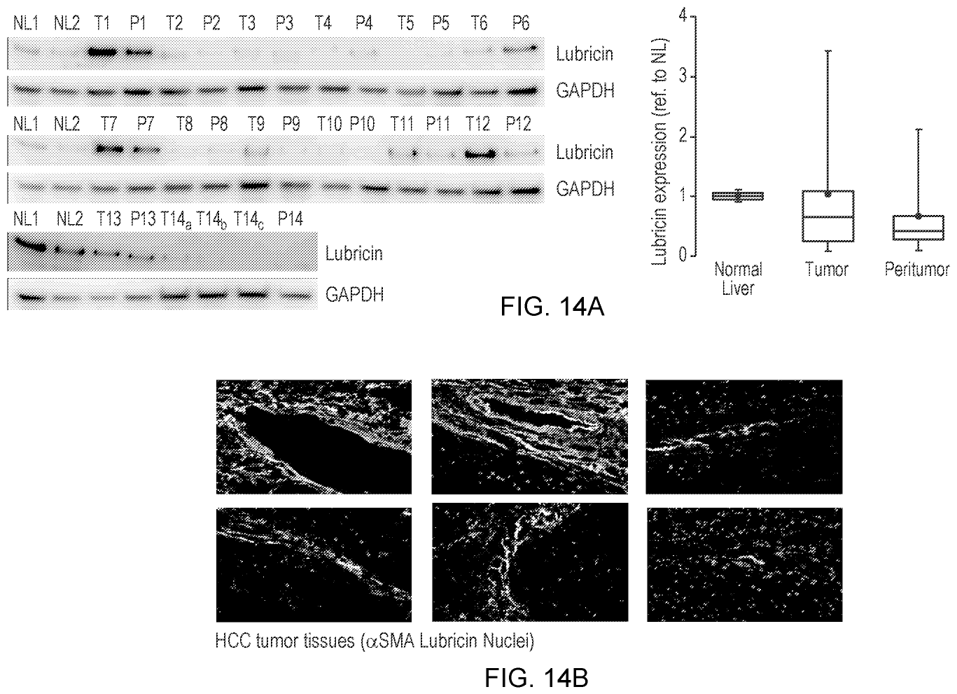

Lubricin is present in HCC tumoral and peritumoral tissues and is expressed under TGFβ stimulation: To further confirm our previous microarray data, by western blotting we investigated the presence of lubricin protein in two normal livers and in 14 tumoral and paired surrounding non-cancerous specimens from HCC patients. Then, we also compared the amount of lubricin in the different specimens. As reported in FIG. 14A, the protein expression levels of lubricin were similar among normal liver, tumoral and peritumoral tissues, although highly variable among different samples. This suggests that the amount of lubricin protein expression is not an epiphenomenon related to tumor development but rather to a unique feature of individual tumoral or peritumoral tissue microenvironments.

To understand the involvement of lubricin in HCC progression, by immunofluorescence, we localized its expression in HCC tissues, where it is mainly distributed in the stromal compartment of the tumor, being mostly detectable in the proximity of alpha smooth muscle actin positive (αSMA+) cells (FIG. 14B).

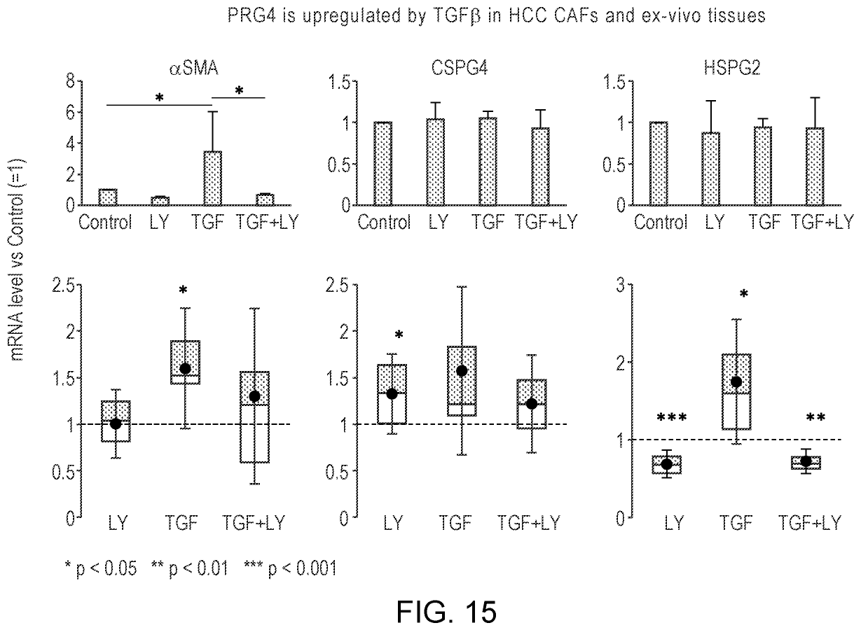

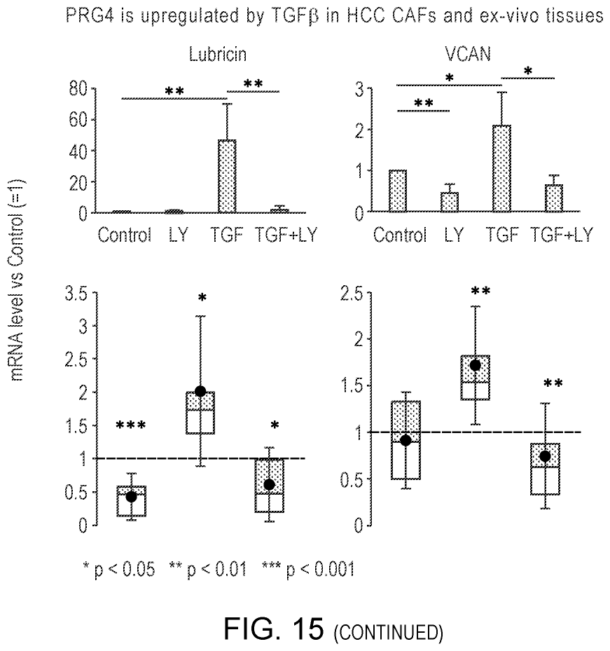

To support the participation of stromal cells in secreting lubricin, we challenged CAFs and ex vivo HCC specimens with TGFβ, the TGFβRI inhibitor, LY2157299 (galunisertib), or both, for 48 hours and then analyzed the PRG4 mRNA expression. TGFβ significantly enhanced the expression of lubricin in both CAFs (p<0.01) and in HCC samples (p<0.05), whereas LY2157299 offset this effect (p<0.01). Interestingly, LY2157299 also downregulated the expression of lubricin in comparison to controls. This probably depends, at least partially, on the blockade of the endogenous TGFβ pathway exerted by this drug. Under the same experimental conditions, we also analyzed mRNA expression of αSMA, CSPG4, HSPG2 and VCAN. Consistently, the myofibroblast phenotype of CAFs was increased by TGFβ (as shown by the enhanced αSMA expression). TGFβ also significantly increased the expression levels of HSPG2 and VCAN in HCC cultured tissues (p<0.05 and p<0.01, respectively), and VCAN but not HSPG2 in CAFs, suggesting that CSPG4 expression is not regulated by TGFβ, and that HSPG2 may be produced in HCC under the control of this cytokine by cells other than CAFs (FIG. 15).

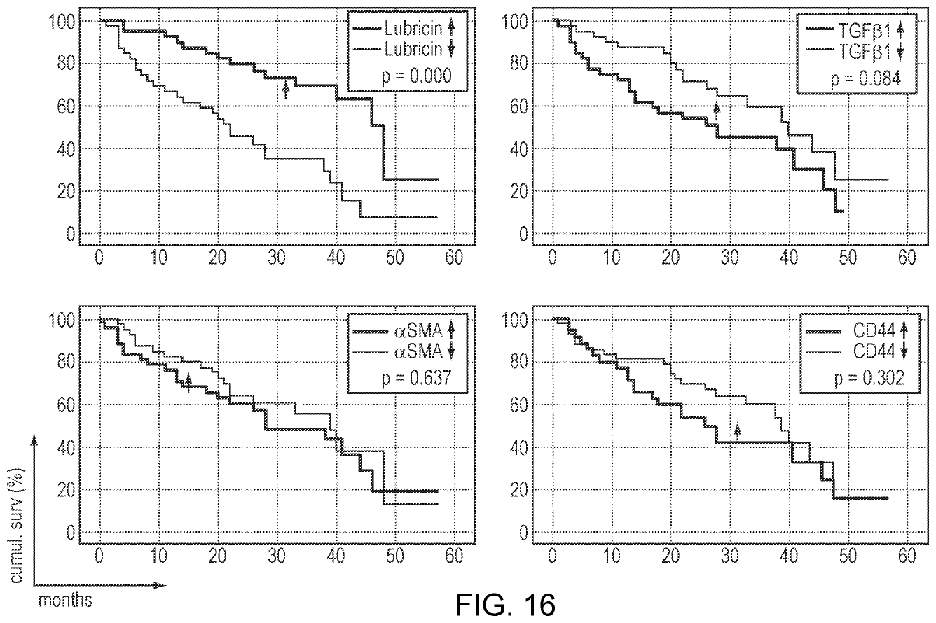

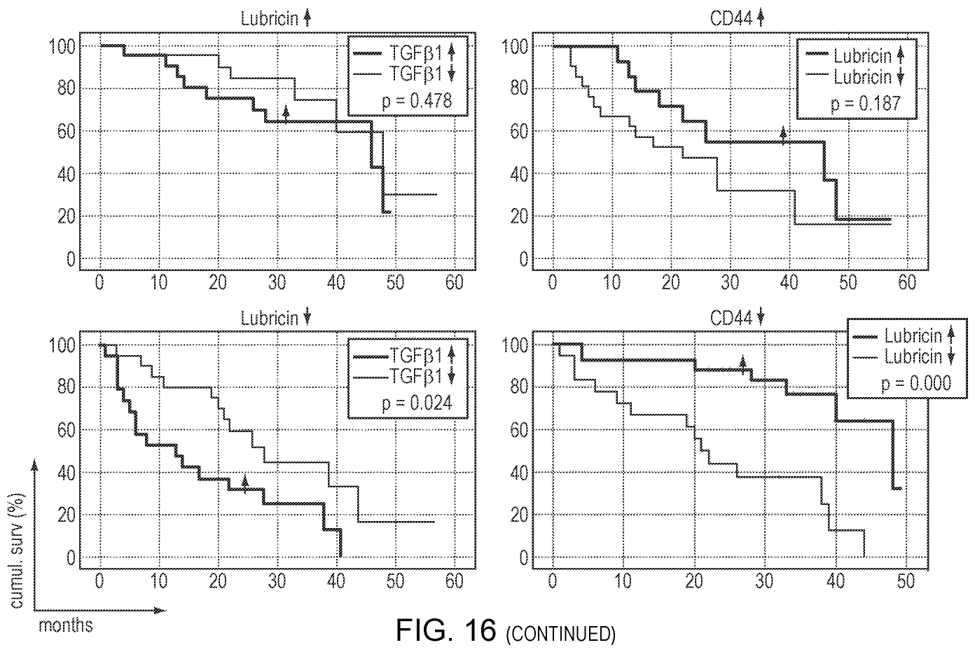

Lubricin is correlated with a better prognosis in HCC patients with lower CD44 expression: To investigate the clinical role of the previously described associations, we stratified the patients according to higher or lower expression levels of lubricin and other related genes in relation to tumor aggressiveness and overall survival. Transcriptomic analysis of lubricin in the tumor demonstrated that lower expression levels were associated with higher biological aggressiveness of the tumor (aggressive vs. bland tumors: 11.1±3.0 vs. 13.4±1.6, p<0.0001). While the increased lubricin expression was significantly (p<0.001) correlated to a better prognosis in patients with lower CD44 expression, TGFβ, CD44 and αSMA expression levels were not associated with any clinical outcome. However, it is noteworthy that in patients with lower lubricin levels, high TGFβ expression was related to a worse prognosis, suggesting that the tumor-suppressing role of TGFβ may be at least partially mediated by its ability to stimulate lubricin expression, whereas any lack of this up-regulation mechanism may result in unleashed TGFβ tumor-promoting actions. Even more interestingly, the putative effect of lubricin as a “protective” factor was lost in the patients with higher CD44 expression (FIG. 16).

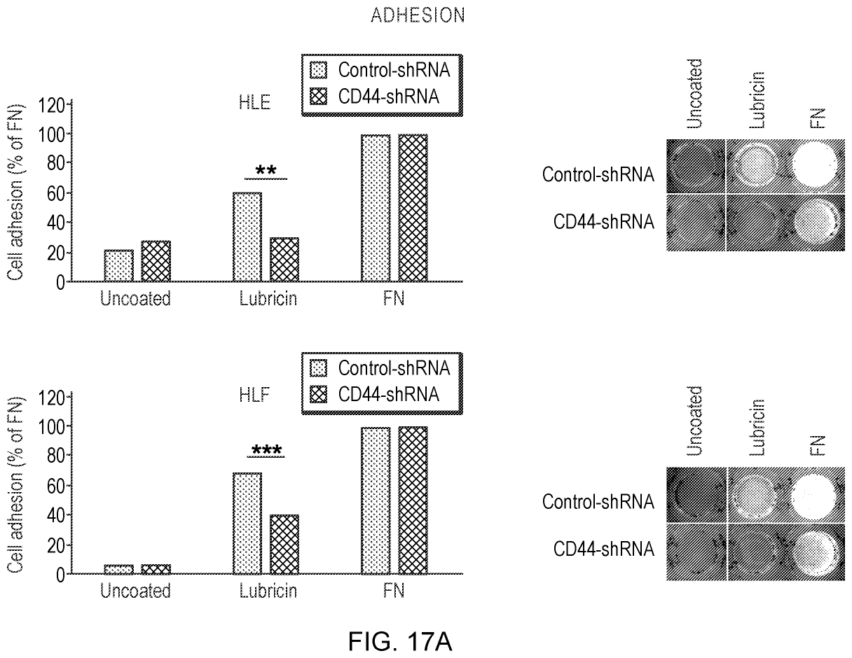

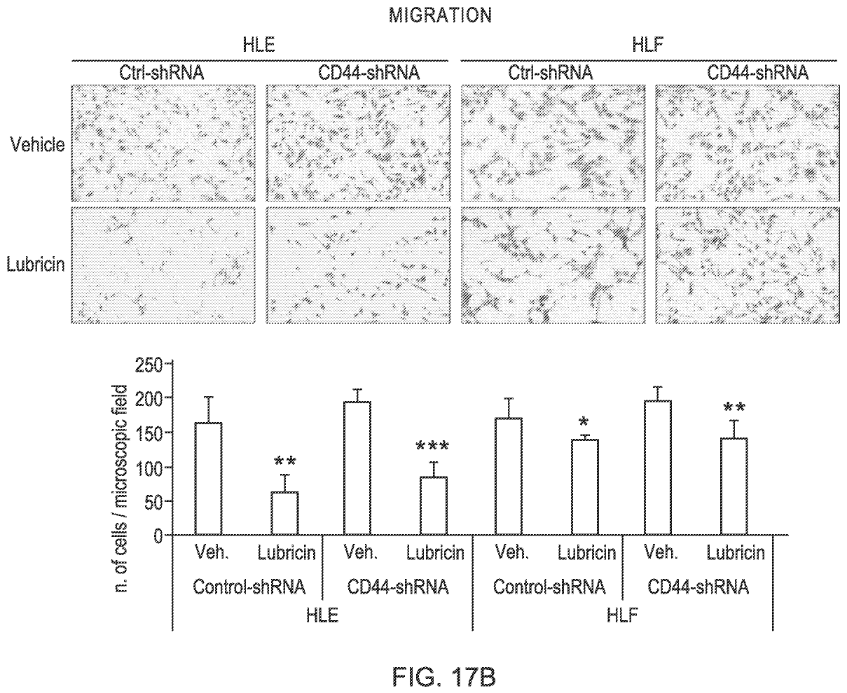

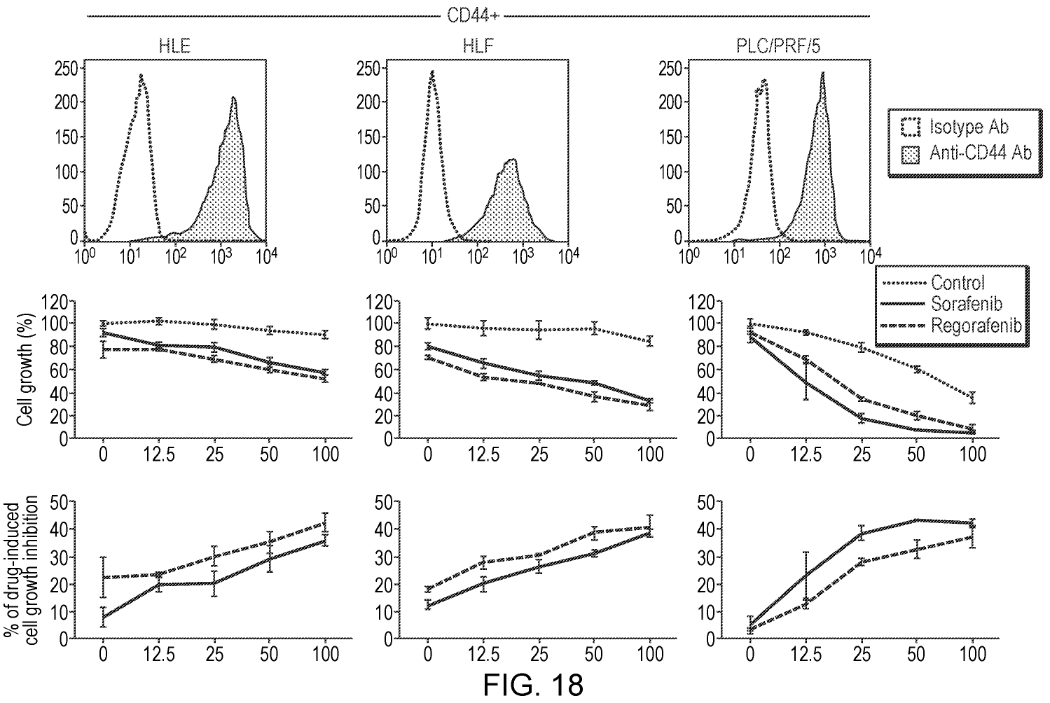

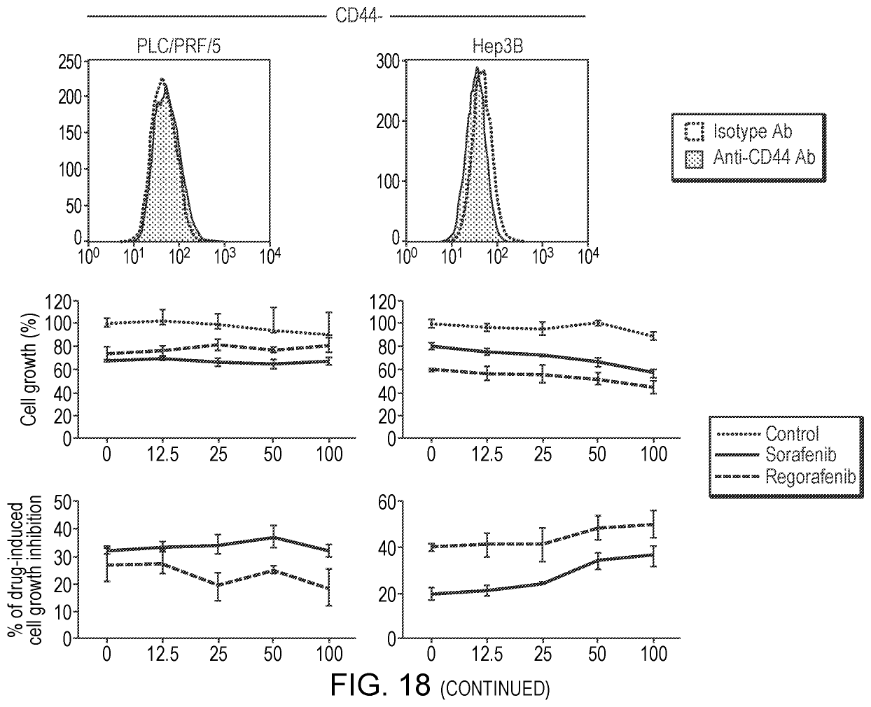

Lubricin inhibits adhesion and migration of HCC cells: To understand how lubricin improves overall survival in HCC patients, and in particular in those with CD44 expression levels below the median value (FIGS. 13 and 16), we investigated whether rhPRG4 affects some key features required for HCC cell aggressiveness, namely adhesion and migration and whether, in turn, the expression of CD44 is implicated. HLE and HLF are two strongly CD44 positive invasive HCC cell lines (as described below, FIG. 18). Adhesion to lubricin is impaired upon silencing of CD44 expression, as regards the adhesion to fibronectin (FIG. 17A). Moreover, soluble rhPRG4 (25 μg/ml) impairs the migration of the same cells on fibronectin, while CD44 downregulation does not affect motility (FIG. 17B). This suggests that the lubricin expressed in the HCC microenvironment might more efficiently saturate CD44 receptor, when it is less expressed, and that other lubricin receptors are likely involved, other than CD44, in limiting cell migration and invasion.

The CD44/lubricin axis boosts Sorafenib and Regorafenib effectiveness on HCC cells: To explore in further detail the biological role of the lubricin/CD44 axis, we challenged HLE, HLF and PLC/DC/19 HCC CD44 positive cells and Hep3B and PLC/PRFS HCC CD44 negative cells with Sorafenib and Regorafenib in the presence/absence of rhPRG4. All HCC cells were grown for 72 hours in the presence/absence of rhPRG4 and Sorafenib or Regorafenib at a concentration (2.5 μM) lower than the IC50 (˜5 μM) under the same experimental conditions. rhPRG4 alone did not markedly impair the cell proliferation but, when coupled with Sorafenib or Regorafenib, it strongly and synergistically improved their effectiveness on HLE, HLF and PLC/DC/19 cells, at concentrations spanning from 12.5 to 100 μg/ml. The rhPRG4-drug synergistic effect was present during Hep3B proliferation, even if lower than that obtained with HLE, HLF and PLC/DC/19 cells, but only at the highest rhPRG4 concentrations. Instead, PLC/PRFS cells did not respond at all (FIG. 18).

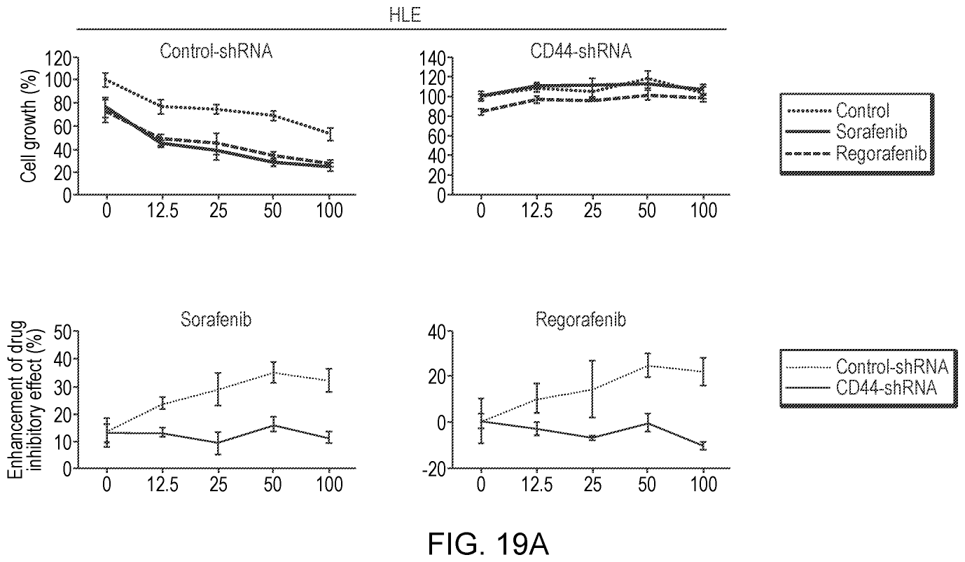

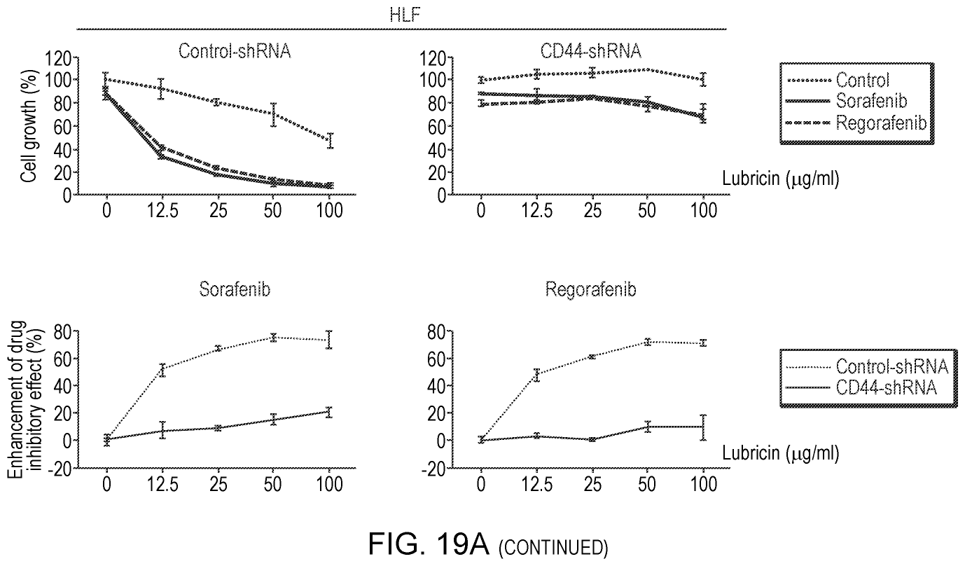

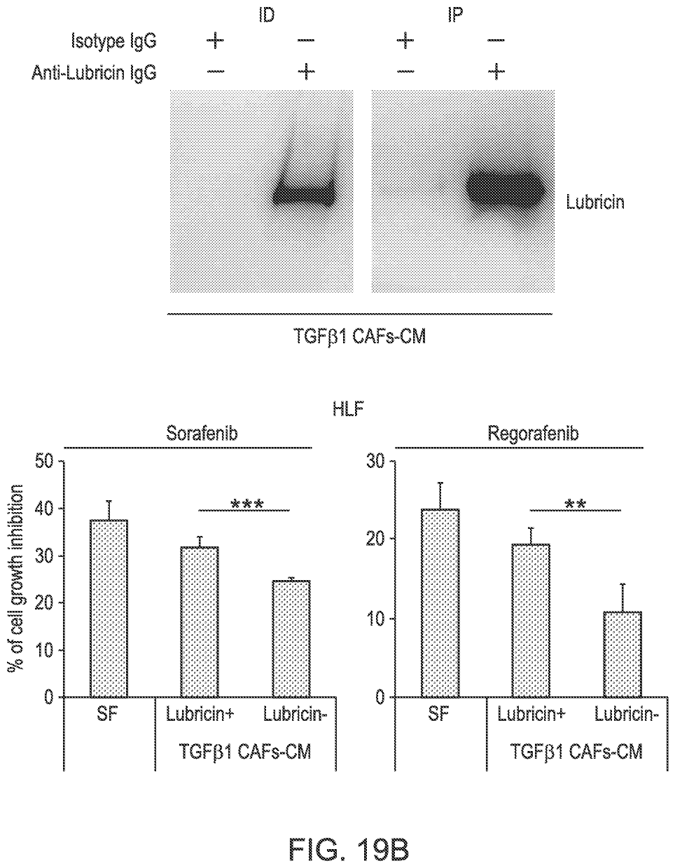

To dissect the molecular mechanism of the CD44/lubricin axis, we used a loss of function approach, silencing CD44 expression in HCC cells via the shRNA lentiviral transduction approach. Control-shRNA and CD44-shRNA HCC cells were tested in a 72-hour proliferation assay in the presence/absence of rhPRG4 (concentrations ranging from 0 to 100 μg/ml), and without or with sorafenib or regorafenib (at concentrations of 2.5 μM). The capacity of both drugs in inhibiting cells proliferation was significantly reduced in CD44 silenced cells (p<0.05 to p<0.001 depending on rhPRG4 concentration), as compared to control cells, demonstrating the need for HCC cells to express CD44 to allow lubricin membrane binding and the subsequent enhanced, drug-induced impairment of HCC cell proliferation in vitro (FIG. 19A). This dampening of the drug effectiveness taking place in rhPRG4-exposed CD44-silenced cells is consistent with the reduced sensitivity to rhPRG4 of drug-exposed low CD44-expressing HCC cell lines PLC/PRF/5 and Hep3B (FIG. 18).

CAFs conditioned medium stimulated by TGFβ increases Sorafenib and Regorafenib effectiveness via lubricin secretion: To further confirm the role of lubricin we reproduced in vitro the biological cascade that resembles a more physiological situation, whereby TGFβ stimulates CAFs that secrete lubricin, improving the drug effectiveness of both Sorafenib and Regorafenib on HCC cells. (FIG. 19B). CAFs were incubated with TGFβ in complete medium for 48 hours and in starving condition (serum free) for additional 48 hours (without TGFβ) to allow the enrichment of secreted proteins pull. The conditioned medium was then collected, incubated with isotype, or anti-lubricin antibody, assayed for protein concentration, and used to challenge HCC cells to proliferate in the presence of 1.5 μM sorafenib and regorafenib and/or 20 μg/ml lubricin-depleted or not-depleted conditioned medium (CM) from TGFβ-stimulated CAFs. The conditioned medium from TGFβ-stimulated CAFs significantly (p<0.01) increased Sorafenib and Regorafenib effectiveness on HLF cells as compared to lubricin-depleted conditioned medium (FIG. 19B). This suggests that the CD44/lubricin axis enhances the drug effectiveness of Sorafenib and Regorafenib.

### Discussion

Until now, cancer cells have always been considered the only reliable therapeutic target, with the few exceptions of those drugs directed against immunological check-points, although the results in terms of clinical outcomes are often unsatisfactory, as in the case of HCC. In this cancer, the cells grow in, and penetrate through, the surrounding tissue derived from the chronic liver disease, that has a rich content of ECM proteins, inflammatory cells and CAFs. In this example, we show that lubricin, a glycoprotein belonging to the PG family, so far known to be present at the cartilage sites of the joints, is expressed in the liver. Moreover, we also demonstrate that its expression is positively correlated to longer survival in a cohort of 78 patients followed up over five years (REF Villa, GUT). In experimental models, we also demonstrate that lubricin inhibits HCC cells adhesion and migration by engaging with the CD44 receptor. This contributes to the understanding of the clinical data. It is also consistent with the demonstration by Sarkar et al. (PLoS One 2019. doi:10.1371/journal.pone.0219697) that lubricin is able to offset the TGFβ-induced enhancement of migration and invasion of breast cancer cell line MDA-MB231, via binding CD44, and thus interfering with its pro-invasive downstream signaling. In addition, these authors found that the expression of CD44 decreases following the binding to lubricin.

CD44 is a sternness marker in HCC, and its expression has been reported to be induced by TGFβ (Fernando et al., Int J Cancer 2015. doi:10.1002/ijc.29097) and, consistently, inhibited by a TGFβ inhibitor (Rani et al. Cell Death Dis 2018. doi:10.1038/s41419-018-0384-5) that also proved effective in the multicentric clinical trial in patients with HCC (Kelley et al., Clin Transl Gastroenterol 2019. doi:10.14309/ctg.0000000000000056). Finally, the CD44/lubricin axis enhances the anti-proliferative action of sorafenib and regorafenib on HCC cells in vitro. To date, lubricin functions have been predominantly known outside the context of cancer. Nahon et al. showed that the absence of lubricin increased the susceptibility to atherosclerosis in two hyperlipidemic mouse models already predisposed to atherosclerosis development, namely apolipoprotein E knockout (ApoE KO) mice and low-density lipoprotein receptor knockout (Ldlr KO) mice (Nahon et al., Atherosclerosis. 2018. doi:10.1016/j.atherosclerosis.2018.06.883). Reduced expression of TGF-β signaling is associated with age-related osteoarthritis in humans. In mice with defective TGF-β signaling, that effectively recapitulate human osteoarthritis, lubricin has proven effective to prevent the onset of this disease, due to its joints lubrication function (Chavez et al., PLoS One 2019. doi:10.1371/journal.pone.0210601).

The interest in the role of lubricin in cancers is growing, especially in light of a recent report that demonstrated the interaction between lubricin and CD44. It has been found that lubricin can compete with hyaluronan for the binding to CD44 and then attenuate the growth-supporting signaling function of this receptor in rheumatoid arthritis fibroblast-like synoviocytes stimulated with interleukin-1β or tumor necrosis factor alpha. (Al-Sharif A et al., Arthritis Rheumatol 2015. doi:10.1002/art.39087).

The emerging anti-tumor role of lubricin may be in apparent contradiction with the evidence that it is up-regulated by TGFβ, as this cytokine is believed to play a dual, opposite role in early and late cancer stages. Indeed, the knowledge accumulated so far suggests that TGFβ signaling behaves as a molecular switch, being cytostatic/pro-apoptotic in the initial steps of solid malignancies, such as HCC, but turning into a metastasis-favoring factor in advanced phases (Moreno-Caceres et al. Cell Death Dis 2017. doi:10.1038/cddis.2017.469; Fabregat et al. TFEBS J 2016. doi:10.1111/febs.13665; Arrese et al., Curr Protein Pept Sci 2017. doi:10.2174/1389203718666171117112619). More specifically, TGFβ signaling can affect CD44 expression/activation, and was shown to rely on CD44 functions to promote cancerous invasion. It has also been demonstrated that TGFβ up-regulates the expression of the CD44 cancer-related CD44V6 isoform through EGR1-mediated AP-1 (activator protein-1) activation in pulmonary fibroblasts (Ghatak et al., J Biol Chem 2017. doi:10.1074/jbc.M116.752451). Nevertheless, other studies have clearly brought to light the capacity of TGFβ to restrain the pro-tumorigenic potential of HCC cells, via inducing the expression of tumor suppressors genes, such as LATS1, or DNA damage repair proteins (ATM, BRCA1, and FANCF) (Zhang et al. Oncotarget 2017. doi:10.18632/oncotarget.14523; Chen et al., Gastroenterology 2018. doi: 10.1053/j.gastro.2017.09.007).

This example suggests that the synergistic anti-proliferative effect arising from a combination of rhPRG4 and sorafenib or regorafenib may result in a significantly improved therapeutic benefit for HCC patients. The strong association of lubricin expression with the overall survival outcome of HCC patients suggests that this molecule may have direct or indirect, not yet elucidated tumor-limiting activities. The finding of no positive correlation between lubricin level and life expectancy in patients with high CD44 expression might reflect a failure of lubricin anti-tumor activity to overcome the pro-malignant function of CD44 signaling, when the expression of this receptor is sufficiently high. Alternatively, different transcription variants of CD44 may be distinctively involved in offsetting drug effectiveness and endogenous stresses. Some of these variants might be unable to bind lubricin, thus resulting in unaffected CD44 downstream signaling. Moreover, there may be competition between these lubricin-insensitive pro-tumor activities of CD44 and tumor-limiting lubricin activities mediated via possible, currently unknown receptors (see FIG. 19A-B).

Overall, this example proposes a novel interventional framework, where CD44 inhibition may be coupled with rhPRG4 administration in high CD44 expression HCCs. Moreover, the potency of sorafenib and regorafenib may be greatly enhanced by a synergic administration with rhPRG4. It has been demonstrated that the efficiency of lubricin binding to CD44 can be significantly increased after the removal of sialic acid and O-glycosylation (Al-Sharif et al., Arthritis Rheumatol 2015. doi:10.1002/art.39087). Therefore, potentially detrimental off-target effects of lubricin could be minimized by using lower concentrations of this processed form. Possible negative effects of lubricin exogenous administration have yet to be carefully evaluated.

In conclusion, we suggest that lubricin has the potential not just to act as an anti-tumor agent, but also to improve the effectiveness of drugs such as sorafenib and regorafenib. Further insights into the molecular mechanisms of lubricin synthesis in the stromal HCC micro-environment, as well as its functional features, possibly gained through identifying novel lubricin-coupling factors, alternative receptors, or partners of interaction, may prove valuable for the purpose of designing more effective pharmacologic tools.

1. A method of treating cancer or slowing the growth or progression of a cancer in a patient comprising administering PRG4 to the patient to treat the cancer or to slow the growth or progression of the cancer.

2. The method of claim 1, wherein the PRG4 comprises:
(a) recombinant human PRG4 comprising the amino acid sequence of residues 25-1404 of SEQ ID NO:1;
(b) an amino acid sequence having at least 99% identity with residues 25-1404 of SEQ ID NO:1; or
(c) an amino acid sequence having at least 99.5% identity with residues 25-1404 of SEQ ID NO:1.

3-4. (canceled)

5. The method of claim 1, wherein the cancer is selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma, Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor.

6-8. (canceled)

9. The method of claim 1, wherein PRG4 is administered in connection with another anti-cancer agent.

10. (canceled)

11. The method of claim 9, wherein the anti-cancer agent is chemotherapy or radiation.

12. The method of claim 11, wherein the chemotherapy is selected from actinomycin, abraxane, altretamine, aranose, azacitidine, azathioprine, bendamustine, bleomycin, bortezomib, busulfan, cabazitaxel, capecitabine, carboplatin, carmofur, carmustine, chlorambucil, chlormethine, chlorozotocin, cisplatin, cladribine, clofarabine, crizotinib, cyclophosphamide, cytarabine, dacarbazine, dactinomycin, dasatinib, daunorubicin, decitabine, docetaxel, doxifluridine, doxorubicin, epirubicin, ertramustine, ethylnitrosourea, erlotinib, etoposide, floxuridine, fludarabine, fluorouracil, fotemustine, gefitinib, gemcitabine, hydroxyurea, hydroxycarbamide, idarubicin, ifosfamide, irinotecan, imatinib, ixabepilone, lapatinib, lomustine, mechlorethamine, melphalan, mercaptopurine, methotrexate, mitomycin, mitoxantrone, nedaplatin, nelarabine, nimustine, nilotinib, N-nitroso-N-methylurea, procarbazine, oxaliplatin, paclitaxel, pemetrexed, pentostatin, ranimustine, raltitrexed, regorafenib, romidepsin, semustine, sorafenib, streptozotocin, tafluposide, tamoxifen, taxotere, tegafur, temozolomide, temsirolimus, teniposide, tioguanine, tofacitinib, opotecan, valrubicin, vemurafenib, vinblastine, vincristine, vincristine, vindesine, vinflunine, vinorelbine, vorinostat, or vismodegib.

13. The method of claim 11, wherein the chemotherapy is an antibody treatment selected from alemtuzumab, bevacizumab, blinatumomab, brentuximab, certolizumab, cetuximab, daratumumab, dinutuximab, ibritumomab, obinutuzumab, ofatumumab, olaratumab, panitumumab, pertuzumab, ramucirumab, rituximab, siltuximab, trastuzumab, rituximab, inotuzumab, gemtuzumab, bevacizumab, camiplimab, or spartalizumab.

14-17. (canceled)

18. The method of claim 1, wherein the PRG4 is administered systemically.

19. (canceled)

20. The method of claim 1, wherein the PRG4 is administered locally to the location of the cancer.

21-22. (canceled)

23. The method of claim 9, wherein the PRG4 enhances chemosensitivity of the cancer to the anti-cancer agent.

24. (canceled)

25. A method for preventing or inhibiting recurrence of a previously treated cancer comprising administering to a patient in need thereof a therapeutically effective amount of PRG4 to prevent recurrence or growth of a previously treated cancer in the patient.

26. The method of claim 25, wherein the PRG4 is administered locally to the site of the cancer that was previously treated.

27-31. (canceled)

32. The method of claim 25, wherein the PRG4 comprises:
(a) recombinant human PRG4 comprising the amino acid sequence of residues 25-1404 of SEQ ID NO:1;
(b) an amino acid sequence having at least 99% identity with residues 25-1404 of SEQ ID NO:1; or
(c) an amino acid sequence having at least 99.5% identity with residues 25-1404 of SEQ ID NO:1.

33-34. (canceled)

35. The method of claim 25, wherein the cancer is selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma, Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor.

36-39. (canceled)

40. The method of claim 25, wherein the patient is in complete or partial remission from the previously treated cancer.

41-42. (canceled)

43. A method of treating cancer comprising administering to a patient in need thereof PRG4 in combination with an immunotherapy agent, wherein the combination of the PRG4 and the immunotherapy treats the cancer.

44. The method of claim 43, wherein the immunotherapy is an anti-PD1 or anti-PD-L1 antibody.

45. The method of claim 44, wherein the immunotherapy is selected from atezolizumab, avelumab, durvalumab, pembrolizumab, nivolumab, cemiplimab, ipilimumab

46. (canceled)

47. The method of claim 43, wherein the cancer is selected from adrenal cancer, anal cancer, bile duct cancer, bladder cancer, bone cancer, brain/CNS cancer, basal cell skin cancer, breast cancer, Castleman disease, cervical cancer, colorectal cancer, endometrial cancer, esophagus cancer, dermatofibrosarcoma protuberans, Ewing family of tumors, eye cancer, gall bladder cancer, gastrointestinal carcinoid tumors, gastrointestinal stromal tumor (GIST), gastric cancer, gestational trophoblastic disease, glioma, glioblastoma, head and neck cancer, hepatocellular carcinoma, Hodgkin disease, Kaposi sarcoma, kidney cancer, laryngeal and hypopharyngeal cancer, leukemia, lung cancer, liver cancer, lymphoma, malignant mesothelioma, Merkel cell carcinoma, melanoma, multiple myeloma, myeloma, myelodysplastic syndrome, nasal cavity and paranasal sinus cancer, nasopharyngeal cancer, neuroendocrine cancer, neuroblastoma, Non-Hodgkin lymphoma, oral cavity and oropharyngeal cancer, osteosarcoma, ovarian cancer, pancreatic cancer, penile cancer, pituitary Tumors, prostate cancer, renal cancer, retinoblastoma, rhabdomyosarcoma, salivary gland cancer, sarcoma, squamous cell skin cancer, small intestine cancer, stomach cancer, testicular cancer, thymus cancer, thyroid cancer, uterine cancer, uterine sarcoma, vaginal cancer, vulvar cancer, Waldenstrom macroglobulinemia, or Wilms tumor.

48-54. (canceled)

55. The method of claim 43, wherein the PRG4 comprises:
(a) recombinant human PRG4 comprising the amino acid sequence of residues 25-1404 of SEQ ID NO:1;
(b) an amino acid sequence having at least 99% identity with residues 25-1404 of SEQ ID NO:1; or
(c) an amino acid sequence having at least 99.5% identity with residues 25-1404 of SEQ ID NO:1.

56-58. (canceled)
